(19) United States
(12) Patent Application Publication
(10) Pub. No.: US 2009/0281278 A1
(43) Pub. Date: Nov. 12, 2009

(54) MODIFIED MOLECULES WHICH PROMOTE HEMATOPOIESIS

(76) Inventors: Hans-Georg Frank, Kerkrade (NL); Udo Haberl, Baesweiler (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/281,565

(22) PCT Filed: Mar. 9, 2007
(86) PCT No.: PCT/EP2007/002068

§ 371 Date: Sep. 16, 2008

Related U.S. Application Data
(60) Provisional application No. 60/780,568, filed on Mar. 9, 2006.
(60) Provisional application No. 60/747,515, filed on May 17, 2006.

(30) Foreign Application Priority Data
Mar. 9, 2006 (EP) 060004833.7
May 17, 2006 (EP) 06010174.8

(51) Int. Cl. C07K 7/06; C07K 7/08

(52) U.S. Cl. 530/327; 530/328

(57) ABSTRACT

The present disclosure relates to modified EPO mimetic peptides having specific properties.

The present invention relates to peptides as binding molecules for the erythropoietin receptor, methods for the preparation thereof, medicaments containing these peptides, and their use in selected indications, preferably for treatment of various forms of anemia and stroke.

The hormone erythropoietin (EPO) is a glycoprotein constituted by 165 amino acids and having four glycosylation sites. The four complex carbohydrate side chains comprise 40 percent of the entire molecular weight of about 35 kD. EPO is formed in the kidneys and from there migrates into the spleen and bone marrow, where it stimulates the production of erythrocytes. In chronic kidney diseases, reduced EPO production results in erythropenic anemia. With recombinant EPO, prepared by genetic engineering, anemias can be treated effectively. EPO improves dialysis patients' quality of life. Not only renal anemia, but also anemia in premature newborns, inflammation and tumor-associated anemias can be improved with recombinant EPO. By means of EPO, a high dosage chemotherapy can be performed more successfully in tumor patients. Similarly, EPO improves the recovery of cancer patients if administered within the scope of radiation therapy.

In the treatment with EPO, a problem exists in that the required dosage regimens are based on frequent or continuous intravenous or subcutaneous applications because the protein is decomposed relatively quickly in the body. Therefore, the evolution of recombinant EPO-derived molecules goes towards selectively modifying the glycoprotein, for example, by additional glycosylation or pegylation, in order to increase stability and thus biological half-life time.

Another important issue associated with the treatment with recombinant EPO is the danger that patients develop antibodies to recombinant EPO during treatment. This is due to the fact that recombinant EPO is not completely identical to endogenous EPO. Once antibody formation is induced, it can lead to antibodies, which compromise the activity of endogenous erythropoietin as well. It frequently increases the dosage of recombinant EPO needed for treatment. Especially if such antibodies compromise the activity of endogenous EPO, this effect can be interpreted as a treatment-induced autoimmune disease. It is especially undesired e.g. in case of dialysis patients undergoing renal transplantation after months or years of EPO-treatment. The antibodies then can compromise the activity of endogenous EPO produced by the transplant and thus compromise erythropoietic activity of the transplanted organ. Presently, it is an open question whether the modifications introduced in recombinant EPO in order to increase biological half-life time will aggravate or improve this problem. Generally, it would be expected that extensive modifications and longer half-life time will aggravate this problematic property.

An alternative strategy is the preparation of synthetic peptides from amino acids which do not share sequence homology or structural relationship with erythropoietin. It was shown that peptides, unrelated to the sequence of EPO, which are significantly smaller than erythropoietin can act as agonists (Wrighton et al., 1996). The same authors showed that such peptides can be truncated to still active minimal peptides with length of 10 amino acids.

Synthetic peptides mimicking EPO's activity are subject of the international laid open WO96/40749. It discloses mimetic peptides of 10 to 40 amino acids of a distinct consensus preferably containing two prolines at the position commonly referred to as position 10 and 17, one of which is considered to be essential. WO 01/38342 discloses that these prolines might be combined with naphthylalanine.

Thus to date, all small peptide-based agonists of the EPO receptor have had a structure which contains at least one proline, often two proline residues in defined positions, usually numbered as position 10 and 17, referenced to their position in the very active erythropoietin-mimetic peptide EMP1 (international laid open WO96/40749; Wrighton et al., 1996, Johnson et al, 1997 and 1998):

GGTYSCHFGPLTWVCKPQGG

These prolines are considered indispensable to the effectiveness of the peptides. For the proline at position 17, this has been substantiated by interactions with the receptor, while the proline at position 10 was thought to be necessary for the correct folding of the molecule (see also Wrighton et al. 1996, 1997). The correct folding, supported by the specific stereochemical properties of proline, is usually a necessary precondition of biological activity. Generally, proline is a structure-forming amino acid which is often involved—as in this case—in the formation of hairpin structures and beta turns. Due to this property, inter alia, it is a frequent point of attack for post-proline-specific endopeptidases which destroy proline-containing peptides/proteins. A number of endogenous peptide hormones (angiotensins I and II, urotensins, thyreoliberin, other liberins, etc.) are inactivated by such “single-hit” post-proline cleavage. Half-life time of proline-containing EPO-mimetic peptides is thus shortened by the activity of these frequent and active enzymes.

Such short peptides can be produced chemically and do not need recombinant production, which is much more difficult to control and to yield products with defined quality and identity. Chemical production of peptides of such small size can also be competitive in terms of production costs. Moreover, chemical production allows defined introduction of molecular variations such as glycosylation, pegylation or any other defined modifications, which can have a known potency to increase biological half-life. However, so far there has been no approval of any therapy with existing EPO mimetic peptides.

Furthermore, there is a need to enhance the EPO mimetic efficacy of the EPO mimetic peptides in order to provide sufficient potent molecules for therapy.

The EPO mimetic peptides described in the state of the art can be regarded as monomeric binding domains recognizing the binding site of the erythropoietin receptor. However, as was pointed out by Wrighton et al. (Wrighton 1997), two of these binding domains are generally needed in order to homodimerize the EPO receptor and to induce signal transduction. Thus, a combination of two of these EPO mimetic peptides and hence the EPO receptor binding domains in one single dimeric molecule enhanced activity considerably. This lead to the result that peptides with one single binding domain showed the same qualitative pattern of activity while two of the binding domains joint together show a much lower ED50 (Effect Dose 50%, a measure of activity). The potency of monomeric EPO mimetic peptides can be improved up to 1000-fold by dimerisation. Even some inactive monomeric peptides can be converted into agonists by dimerization. Peptides harboring two binding domains are specified as being bivalent or dimeric peptides.

Several techniques are known to dimerize the monomers. Monomers can be dimerized e.g. by covalent attachment to a linker. A linker is a ioining molecule creating a covalent bond between the polypeptide units of the present invention. The polypeptide units can be combined via a linker in such a way, that the binding 5 to the EPO receptor is improved (Johnson et al. 1997; Wrighton et al. 1997). It is furthermore referred to the multimerization of monomeric biotinylated peptides by non-covalent interaction with a protein carrier molecule described by Wrighton et al (Wrighton, 1997). It is also possible to use a biotin/streptavidin system i.e. biotinylating the C-terminus of the peptides and a subsequent incubating the biotinylated peptides with streptavidin. Alternatively, it is known to achieve dimerization by forming a diketopiperazine structure. This method known to the skilled person is described in detail e.g. in Cavelier et al. (in: Peptides: The wave of the Future; Michal Lebl and Richard A. Houghten (eds); American Peptide Society, 2001). Another alternative way to obtain peptide dimers known from prior art is to use bifunctional activated dicarboxylic acid derivatives as reactive precursors of the later linker moieties, which react with N-terminal amino groups, thereby forming the final dimeric peptide (Johnson et al, 1997). Monomers can also be dimerized by covalent attachment to a linker. Preferably the linker comprises NH—R—NH wherein R is a lower alkylene substituted with a functional group such as carboxyl group or amino group that enables binding to another molecule moiety. The linker might contain a lysine residue or lysine amide. Also PEG may be used a linker. The linker can be a molecule containing two carboxylic acids and optionally substituted at one or more atoms with a functional group such as an amine capable of being bound to one or more PEG molecules. A detailed description of possible steps for oligomerization and dimerization of peptides with a linking moiety is also given in WO 2004/101606. Alternative dimerisation strategies for EPO mimetic peptides are appreciated.

Furthermore, it should be noted that EPO and EPO mimetic peptides (monomeric or dimeric) are not only interesting for human therapeutic purposes. Beyond human applications there is a great need for EPO substitutes in the animal health care market. In this respect it is desirable to provide EPO mimetic peptides showing a discriminating activity pattern in humans and animals in order to prevent abuse. This is however, a challenging task, since the sequences of different animal EPO receptors (e.g. mouse, rat, pig and dog) are very similar to the human EPO receptor. When aligning the EPO receptors of different species it becomes clear that the different species differ in only a few amino acids. This implicates a high structural homology. Furthermore, only a small percentage of these amino acid residues are relevant for binding to EPO mimetic peptides. This aggravates the development of an EPO mimetic peptide depicting different levels of activity on the human and the animal EPO receptors.

It is an object of the present invention to provide alternative synthetic peptides which exhibit at least essential parts of the biological activity of the native EPO and thus provide alternative means for efficient therapeutic strategies.

It is a further object of the present invention to provide EPO mimetic peptides with an improved efficacy.

It is a further object of the present invention to provide dimers of EPO mimetic peptides by alternative dimerisation strategies.

Furthermore, it is an object of the present invention to provide EPO mimetic peptides which depict a diverging activity pattern in humans and animals.

The solutions to these objects will be outlined in detail below.

According to a first embodiment of the invention, a peptide is provided, especially one being capable of binding the EPO receptor, comprising the following consensus sequence of amino acids:

X₆X₇X₈X₉X₁₀X₁₁X₁₂X₁₃X₁₄X₁₅

wherein each amino acid is selected from natural or unnatural amino acids and
X₆ is an amino acid with a sidechain functionality capable of forming a covalent bond or A or α-amino-γ-bromobutyric acid;
X₇ is R, H, L, W, Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is an uncharged polar amino acid or A;
X₁₃ W, 1-nal, 2-nal, A or F;
X₁₄ is D, E, I, L or V;
X₁₅ is an amino acid with a sidechain functionality capable of forming a covalent bond or A.

Also comprised by this embodiment are peptides selected from the group consisting of functionally equivalent fragments, derivatives and variants of the above peptide consensus sequence, having EPO mimetic activity and having an amino acid in position X₁₀ that constitutes a non-conservative exchange of proline or wherein X₉ and X₁₀ are substituted by a single amino acid.

The described peptide consensus sequences can be perceived as monomeric binding domains for the EPO receptor. As EPO mimetic peptides they are capable of binding to the EPO receptor.

The length of the peptide is preferably between ten to forty or fifty or sixty amino acids. In preferred embodiments, the peptide consensus depicts a length of at least 10, 15, 18, 20 or 25 amino acids. Of course the consensus can be embedded respectively be comprised by longer sequences. A longer length can also be created by dimerising two monomeric peptide units of the above consensus (see below).

It was very surprising that the peptides according to the invention do exhibit EPO mimetic activities although one or—according to some embodiments—even both prolines of the known EPO mimetic peptides according to Wrighton and Johnson are replaced by other natural or non-natural amino acids. In fact the peptides according to the invention have an activity comparable or even better to that of the known proline-containing peptides. However, it is noteworthy that the amino acids substituting proline residues do not represent a conservative exchange but instead a non-conservative exchange of proline. Suitable examples of such non-conservative exchanges of proline are positively or negatively charged amino acids in position 10.

Preferably, a positively charged amino acid such as basic amino acids such as e.g. the proteinogenic amino acids K, R and H and especially K can be used for substitution. The non-conservative amino acid used for substitution of the proline in position 10 can also be a non-proteinogenic natural or a non-natural amino acid and is preferably one with a positively charged side chain. Also comprised are respective analogues of the mentioned amino acids. Non-proteinogenic positively charged amino acids having a side chain which is elongated compared to lysine proved to be especially active. A suitable example of such an elongated amino acid is homoarginine. According to one embodiment the peptide carries a positively charged amino acid in position 10 except for the natural amino acid arginine. According to this embodiment the proline 10 is substituted by a positively charged amino acid selected from the group consisting of proteinogenic amino acids K or H and positively charged non-proteinogenic natural and non-natural positively charged amino acids such as e.g. homoarginine.

According to the consensus sequence of the first embodiment, X₆ and X₁₅ depict amino acids with a sidechain functionality capable of forming a covalent bond. These amino acids are thus capable of forming a bridge unit. According to one embodiment, the amino acids in position X₆ and X₁₅ are chosen such that they are capable of forming an intramolecular bridge within the peptide by forming a covalent bond between each other. Forming of an intramolecular bridge may lead to cyclisation of the peptide. Examples for suitable bridge units are the disulfide bridge and the diselenide bridge. Suitable examples of amino acids depicting such bridge forming functionalities in their side chains are e.g. cysteine and cysteine derivatives such as homocysteine or selenocysteine but also thiolysine. The formation of a diselenide bridge e.g. between two selenocysteine residues even has advantages over a cysteine bridge. This as a selenide bridge is more stable in reducing environments. The conformation of the peptide is thus preserved even under difficult conditions.

However, it is evident that also amino acids are suitable in position X₆ and X₁₅, depicting a side chain with a functionality allowing the formation of different covalent bonds such as e.g. an amide bond between an amino acid having a positively charged side chain (e.g. the proteinogenic amino acids K, H, R or ornithine, DAP or DAB) and an amino acid having a negatively charged side chain (e.g. the proteinogenic amino acids D or E). Further examples are amide and thioether bridges.

Peptides falling under the consensus sequence of the first embodiment of the present invention are disclosed in applicant's earlier application PCT/EP2005/012075 (WO 2006/050959), which was published after the priority-dates of the present application. In some countries this disclosure in the PCT/EP 2005/012075 might constitute prior art according to the respective patent law.

Only in countries where this is applicable and could question patentability, the consensus sequence described above for legal reasons may not comprise sequences fulfilling the above consensus that are disclosed in PCT EP 2005 01 20 75. This could apply to the following consensus sequences or peptide sequences:
a peptide, especially one being capable of binding the EPO receptor comprising the following sequence of amino acids:

X₆X₇X₈X₉X₁₀X₁₁X₁₂X₁₃X₁₄X₁₅

wherein each amino acid is selected from natural or unnatural amino acids and
X₆ is C, A, E, α-amino-γ-bromobutyric acid or homocysteine (hoc);
X₇ is R, H, L, W, Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is T or A;
X₁₃ W, 1-nal, 2-nal, A or F;
X₁₄ is D, E, I, L or V;
X₁₅ is C, A, K, α-amino-γ-bromobutyric acid or homocysteine (hoc) provided that either X₆ or X₁₅ is C or hoc;
a peptide, characterised by the following sequence of amino acids:

X₆X₇X₈X₉X₁₀X₁₁X₁₂X₁₃X₁₄X₁₅

wherein each amino acid is indicated by standard letter abbreviation and
X₆ is C;
X₇ is R, H, L or W;
X₈ is M, F or I;
X₉ is G or a conservative exchange of G;
X₁₀ is a non conservative exchange of proline;
X₁₁ is independently selected from any amino acid;
X₁₂ is T;
X₁₃ is W;
X₁₄ is D, E, I, L or V;
X₁₅ is C;
or wherein X₉ and X₁₀ are substituted by a single amino acid
a peptide is characterised by the following amino acid sequence:

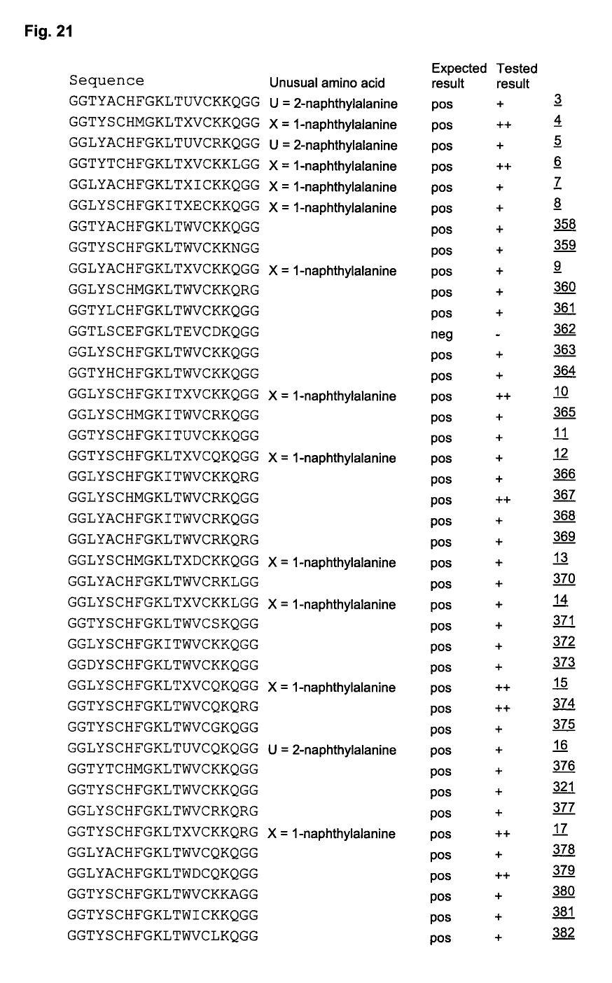

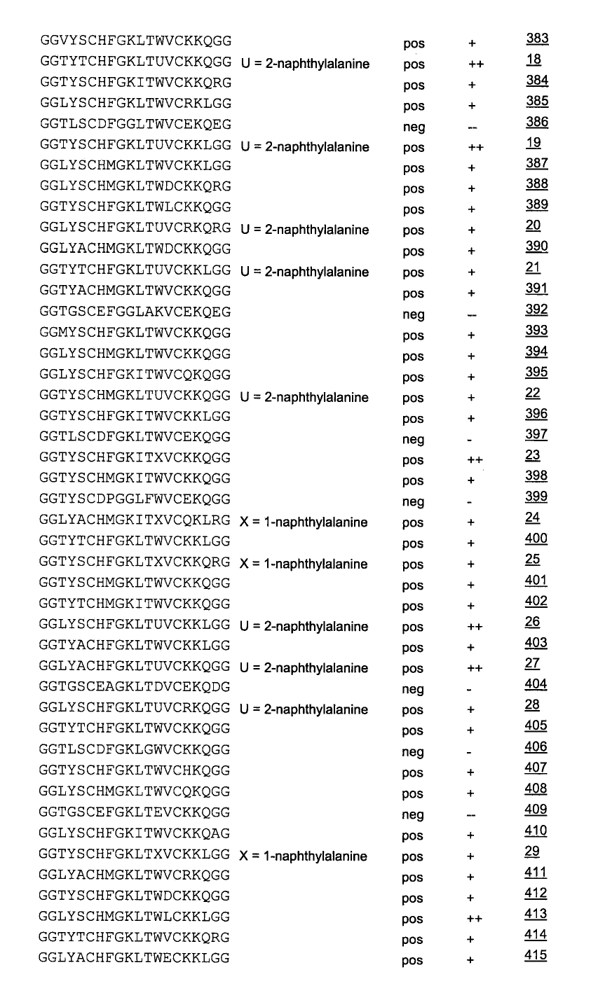

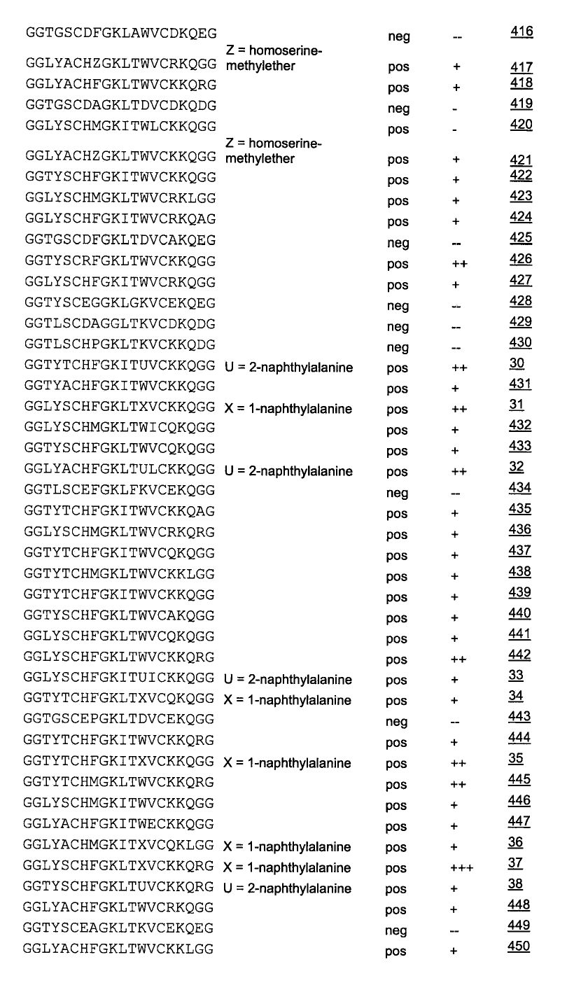

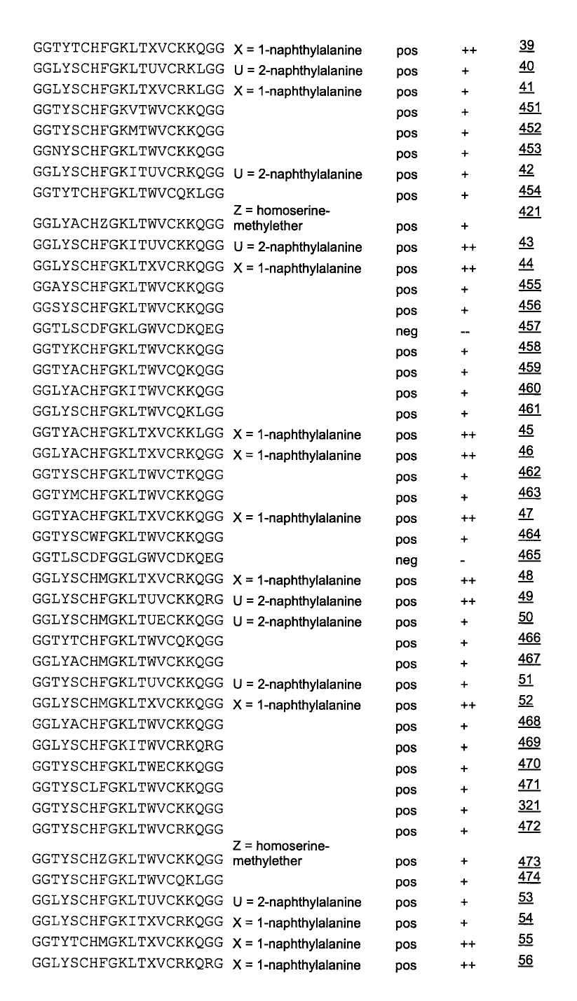

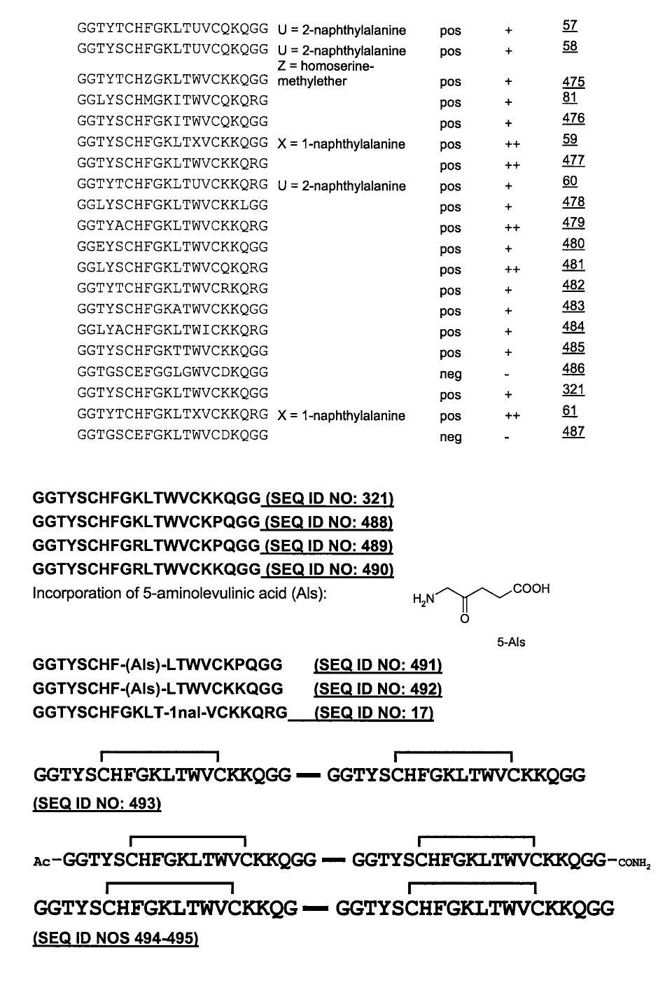

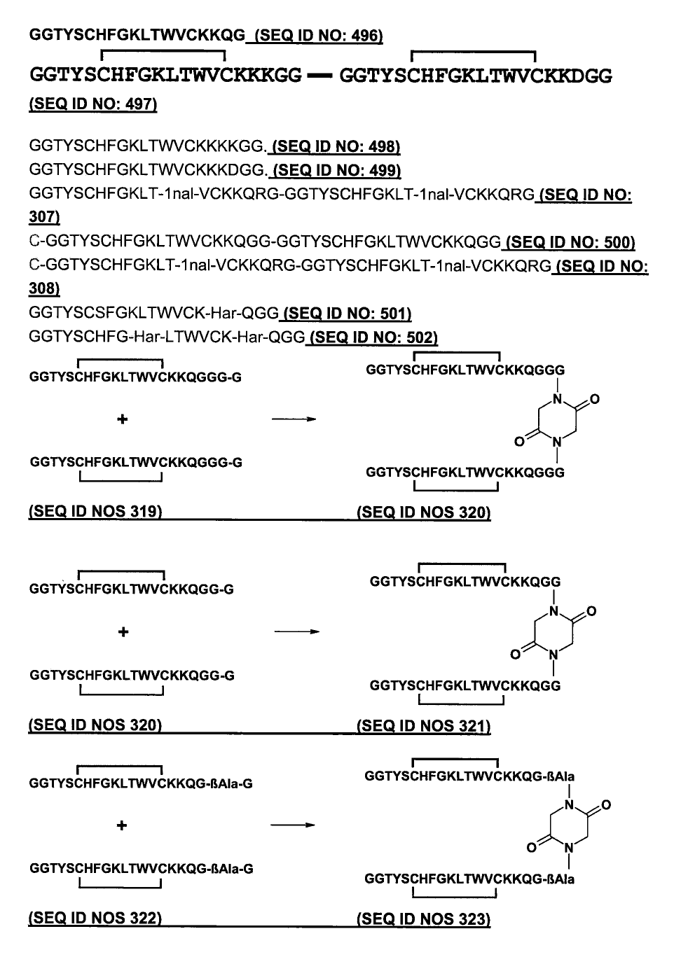

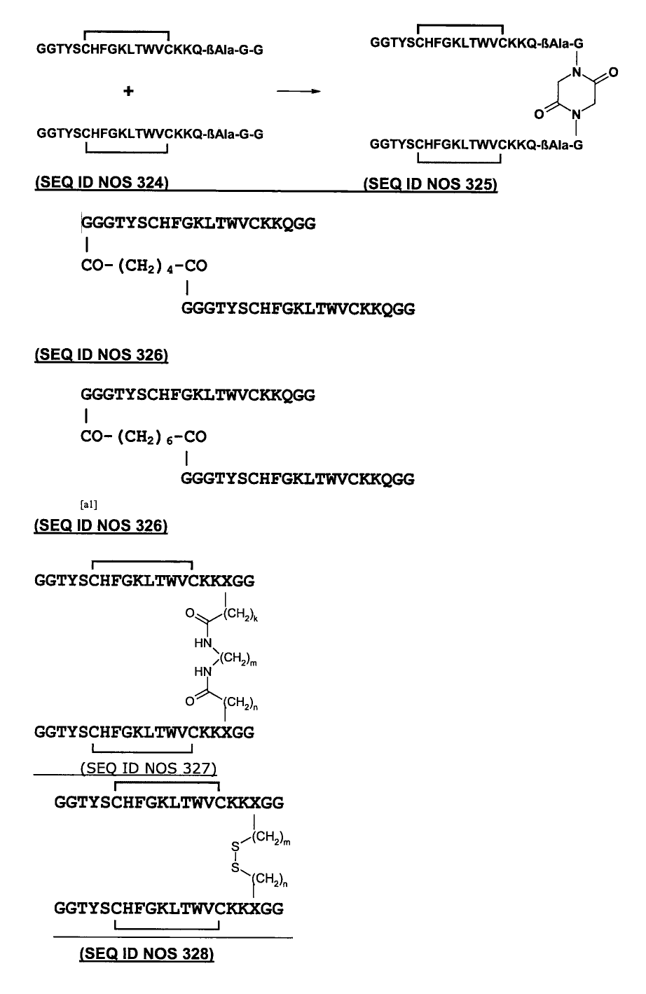

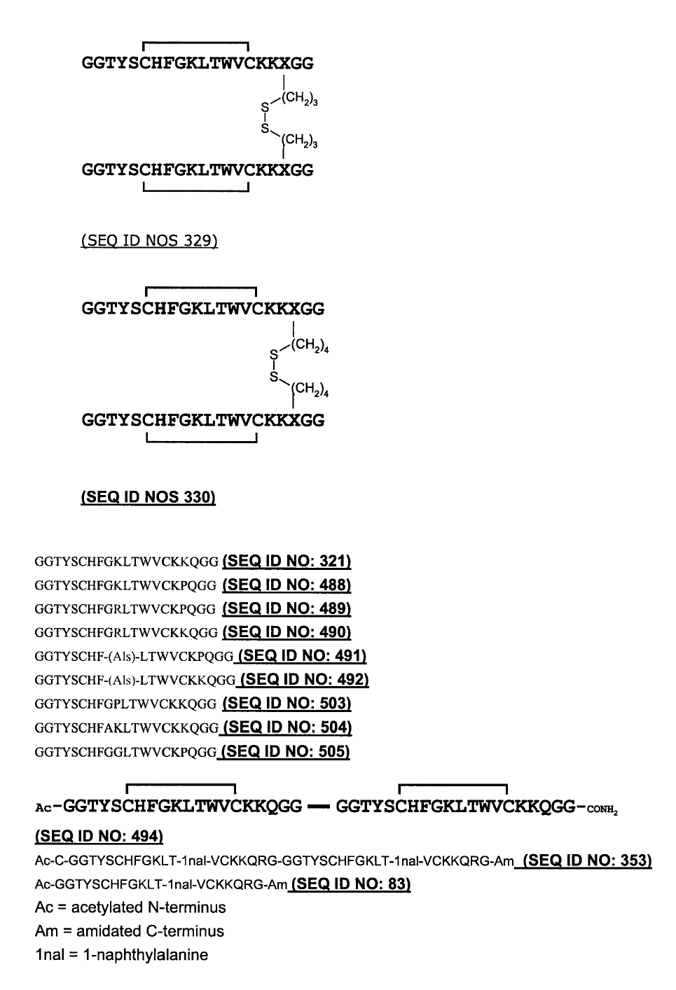

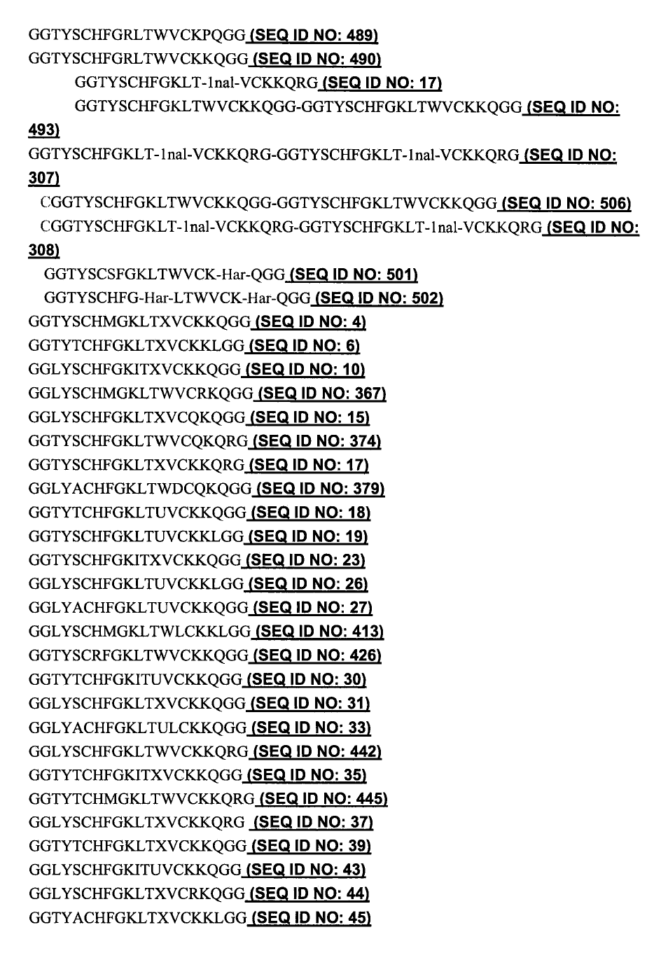

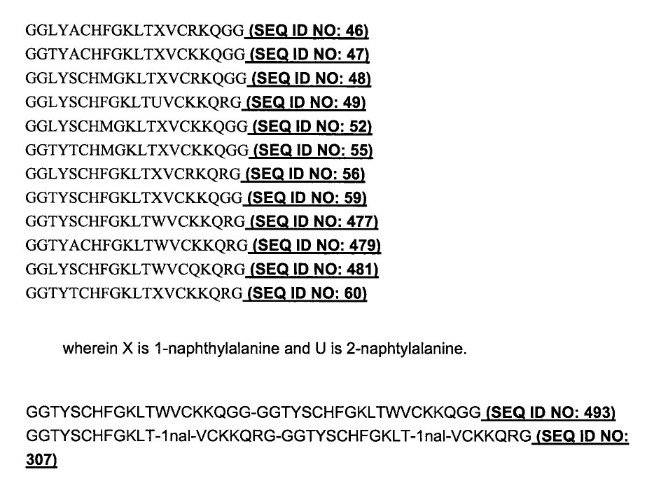

X₆X₇X₈X₉X₁₀X₁₁X₁₂X₁₃X₁₄X₁₅

wherein each amino acid is indicated by standard letter abbreviation and
X₆ is C;
X₇ is R, H, L or W;
X₈ is M, F, I, or hsm (homoserine methylether);
X₉ is G or a conservative exchange of G;
X₁₀ is a non conservative exchange of proline;
X₁₁ is independently selected from any amino acid;
X₁₂ is T;
X₁₃ is W;
X₁₄ is D, E, I, L or V, 1-nal (1-naphthylalanine) or 2-nal (2-naphtylalanine);
X₁₅ is C;
   the peptides disclosed in PCT/EP2005/012075 fulfilling the above consensus of the first embodiment (see FIG. 21).

Where the earlier postpublished disclosure of PCT/EP2005/012075 does not result in a patentability problem, the above listed consensus and peptide sequences need not to be disclaimed from the broad consensus of the first embodiment and are thus comprised by the above defined consensus. Furthermore, these peptides support the accurateness of the EPO mimetic consensus in general as they demonstrate the effectiveness.

According to a second embodiment of the present invention, a peptide is provided, which also depicts good EPO mimetic properties. This peptide comprises at least 10 amino acids, is capable of binding to the EPO receptor and comprises an agonist and thus EPO mimetic activity. Said peptide comprises the following core sequence of amino acids:

X₉X₁₀X₁₁X₁₂X₁₃

wherein each amino acid is selected from natural or non-natural amino acids, and wherein:
X₉ is G or a conservative exchange of G;
X₁₀ is a non conservative exchange of proline or X₉ and X₁₀ are substituted by a single amino acid;
X₁₁ is selected from any amino acid;
X₁₂ is an uncharged polar amino acid or A;
X₁₃ is naphthylalanine.

Also comprised by this embodiment are peptides selected from the group consisting of functionally equivalent fragments, derivatives and variants of the above peptide consensus sequence, that have EPO mimetic activity and having an amino acid in X₁₀ that constitutes a non-conservative exchange of proline or wherein X₉ and X₁₀ are substituted by a single amino acid and which depict a naphthylalanine in position X₁₃.

The peptides of this second embodiment share with the first embodiment the unique feature that X₁₀ is a non conservative exchange of proline or that X₉ and X₁₀ are substituted by a single amino acid. However, a further characteristic for the EPO mimetic peptides according to the second embodiment of the present invention is the naphthylalanine (e.g. either 1-NaI or 2-NaI) in position 13.

The combination of naphthylalanine in position 13 and the non-conservative amino acid exchange of proline in position X₁₀ leads to EPO mimetic peptides with improved binding properties.

EPO mimetic peptides bind in form of a dimer to the EPO receptor. We assume that the incorporation of NaI in position 13 leads to stronger hydrophobic interactions between the peptide monomers. This potentially enhances the dimerisation of the monomeric peptide chains and possibly stabilises the conformation of the peptide dimer. In combination with an amino acid which is non-conservative to proline, an EPO mimetic molecule with improved EPO mimetic properties is created, maybe due to a favourable placement of the amino acids involved in receptor binding.

Sequences depicting naphthylalanine in position 13 were also disclosed in applicant's earlier application PCT/EP 2005/012075. In some countries this disclosure might constitute prior art according to the patent law.

In countries where this is applicable and could question patentability, the consensus sequence of the first alternative of the second embodiment for legal reasons may not comprise sequences fulfilling the consensus that are disclosed in PCT/EP 2005/012075. This could apply to the following consensus sequence and peptide sequences selected from the following group that are disclosed in PCT/EP 2005/012075:
a peptide, especially one being capable of binding the EPO receptor comprising the following sequence of amino acids:

X₆X₇X₈X₉X₁₀X₁₁X₁₂X₁₃X₁₄X₁₅

wherein each amino acid is selected from natural or unnatural amino acids and
X₆ is C, A, E, α-amino-γ-bromobutyric acid or homocysteine (hoc);
X₇ is R, H, L, W or Y or R, H, L, W, Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is T or A;
X₁₃ is 1-nal, 2-nal;
X₁₄ is D, E, I, L or V;
X₁₅ is C, A, K, α-amino-γ-bromobutyric acid or homocysteine (hoc) provided that either X₆ or X₁₅ is C or hoc;
   a peptide of the following group

GGTYSCHFGKITUVCKKQGG
GGTYSCHFGKLT-1nal-VCKKQRG
GGTYSCHFGKLT-1nal-VCKKQRG-GGTYSCHFGKLT-1nal-
VCKKQRG
C-GGTYSCHFGKLT-1nal-VCKKQRG-GGTYSCHFGKLT-1nal-
VCKKQRG
Ac-C-GGTYSCHFGKLT-1nal-VCKKQRG-GGTYSCHFGKLT-1nal-
VCKKQRG-Am
Ac-GGTYSCHFGKLT-1nal-VCKKQRG-Am
GGTYSCHFGKLT-1nal-VCKKQRG
GGTYSCHFGKLT-1nal-VCKKQRG-GGTYSCHFGKLT-1nal-
VCKKQRG
CGGTYSCHFGKLT-1nal-VCKKQRG-GGTYSCHFGKLT-1nal-
VCKKQRG
GGTYSCHMGKLTXVCKKQGG
GGTYTCHFGKLTXVCKKLGG
GGLYSCHFGKITXVCKKQGG
GGLYSCHFGKLTXVCQKQGG
GGTYSCHFGKLTXVCKKQRG
GGTYTCHFGKLTUVCKKQGG
GGTYSCHFGKLTUVCKKLGG
GGTYSCHFGKITXVCKKQGG
GGLYSCHFGKLTUVCKKLGG
GGLYACHFGKLTUVCKKQGG
GGTYTCHFGKITUVCKKQGG
GGLYSCHFGKLTXVCKKQGG
GGLYACHFGKLTULCKKQGG
GGTYTCHFGKITXVCKKQGG
GGLYSCHFGKLTXVCKKQRG
GGTYTCHFGKLTXVCKKQGG
GGLYSCHFGKITUVCKKQGG
GGLYSCHFGKLTXVCRKQGG
GGTYACHFGKLTXVCKKLGG
GGLYACHFGKLTXVCRKQGG
GGTYACHFGKLTXVCKKQGG
GGLYSCHMGKLTXVCRKQGG
GGLYSCHFGKLTUVCKKQRG
GGLYSCHMGKLTXVCKKQGG
GGTYTCHMGKLTXVCKKQGG
GGLYSCHFGKLTXVCRKQRG
GGTYSCHFGKLTXVCKKQGG
GGTYTCHFGKLTXVCKKQRG
GGTYTCHFGKLTXVCKKQRG
GGTYACHFGKLTUVCKKQGG
GGLYACHFGKLTUVCRKQGG
GGLYACHFGKLTXTCKKQGG
GGLYSCHFGKITXECKKQGG
GGLYACHFGKLTXVCKKQGG
GGTYSCHFGKLTXVCQKQGG
GGLYSCHMGKLTXDCKKQGG
GGLYSCHFGKLTXVCKKLGG
GGLYSCHFGKLTUVCQKQGG
GGLYSCHFGKLTUVCRKQRG
GGTYTCHFGKLTUVCKKLGG
GGTYSCHMGKLTUVCKKQGG
GGLYACHMGKITXVCQKLRG
GGTYSCHFGKLTXVCKKQRG
GGLYSCHFGKLTUVCRKQGG
GGTYSCHFGKLTXVCKKLGG
GGLYSCHFGKITUICKKQGG
GGTYTCHFGKLTXVCQKQGG
GGLYACHMGKITXVCQKLGG
GGTYSCHFGKLTUVCKKQRG
GGLYSCHFGKLTUVCRKLGG
GGLYSCHFGKLTXVCRKLGG
GGLYSCHFGKTTUVCRKQGG
GGLYSCHMGKLTUECKKQGG
GGTYSCHFGKLTUVCKKQGG
GGLYSCHFGKLTUVCKKQGG
GGLYSCHFGKITXVCRKQGG
GGTYTCHFGKLTUVCQKQGG
GGTYSCHFGKLTUVCQKQGG
GGTYTCHFGKLTUVCKKQRG

wherein X is 1-naphthylalanine and U is 2-naphthylalanine.

Where the postpublished disclosure of PCT/EP2005/012075 does not result in a patentability problem, the above listed consensus and peptide sequences need not to be disclaimed from the broad consensus of the first alternative of the second embodiment and are thus comprised by the above defined broad consensus.

Further beneficial aspects of the first and second embodiment of the present invention are provided in the dependent claims. As the first and second embodiment of this invention share identical features regarding the presence of a non-conservative exchange of proline in position 10 or in that X₉ and X₁₀ are substituted by a single amino acid, they in fact are tightly linked to each other.

Enlarged consensus sequences of the first and second embodiment, wherein suitable amino acids are defined for positions surrounding the above core sequences are defined in the dependent claims and are also described below. Please note that the numbering used in the present application (X₄X₅X₆ . . . etc) is only provided in order to alleviate the comparison between the peptides of the present invention and the EPO mimetic peptides known in the state of the art (for numbering based on the EMP1 peptide please refer e.g. Johnson et al, 1997 and 1998). However, this numbering does not refer to the overall length of the peptide and hence shall also not imply that it is always necessary that all positions are occupied. It is e.g. not necessary that position X₁ is occupied. E.g. a peptide starting with X₆ is also EPO mimetically active as long as the minimal length of 10 amino acids is provided. Consequently, the numbering of the amino acid positions used in this application shall only alleviate the characterisation and comparison of the peptides with the prior art.

The consensus sequence of the first and second embodiment of the present invention may also comprise the following additional amino acid positions:

X₁₄X₁₅X₁₆X₁₇X₁₈X₁₉

wherein each amino acid is selected from natural or unnatural amino acids and
X₁₄ is selected from the group consisting of D, E, I, L or V;
X₁₅ is an amino acid with a sidechain functionality capable of forming a covalent bond or A;
X₁₆ is independently selected from any amino acid, preferably G, K, L, Q, R, S, Har or T;
X₁₇ is selected from any amino acid, preferably A, G, P, Y or a positively or negatively charged natural, non-natural or derivatized amino acid, in case of a positively charged amino acid preferably K, R, H, ornithine or homoarginine;
X₁₈ is independently selected from any amino acid, preferably L or Q;
X₁₉ is independently selected from any amino acid, preferably a positively or negatively charged amino acid, in case of a positively charged amino acid e.g. K, R, H, ornithine or homoarginine or a small flexible amino acid such as glycine or beta-alanine.

According to a further improvement, the peptide consensus comprises the following additional amino acid positions:

X₆X₇X₈

wherein each amino acid is selected from natural or non-natural amino acids and wherein
X₆ is an amino acid with a sidechain functionality, capable of forming a covalent bond or A or α-amino-γ-bromobutyric acid;

### X₇ is R, H, L, W or Y or S;

X₈ is M, F, I, Y, H, homoserinemethylether or norisoleucine.

According to a further improvement of the first and second embodiment of the invention, the peptide depicts a charged amino acid in position X₁₀, X₁₇ and/or X₁₉ if these amino acid positions are present in the peptide (depends on the length of the peptide consensus). The amino acids in position X₁₀, X₁₇ and/or X₁₉ are either positively or negatively charged and are selected from the group consisting of natural amino acids, non-natural amino acids and derivatized amino acids. Please note that derivatized amino acids are perceived as a special embodiment of non-natural amino acids in the context of this application. The term non-natural amino acid is in fact the generic term. Derivatised amino-acids are presently separately mentioned, since they constitute a special embodiment of the present invention as will be described in detail below.

In case the amino acids in X₁₀, X₁₇ and/or X₁₉ are negatively charged amino acids, said negatively charged amino acids are preferably selected from the group consisting of
natural negatively charged amino acids, especially D or E;
non-natural negatively charged amino acids,
originally positively charged amino acids which are, however, derivatized with suitable chemical groups in order to provide them with a negatively charged group.

The non-natural negatively charged side chain may depict an elongated side chain. Examples for such amino acids are alpha-amino adipic acid (Aad), 2-aminoheptanediacid (2-aminopimelic acid) or alpha-aminosuberic acid (Asu).

One reason might be that the elongated negatively charged artificial amino acids are capable to get in better contact with positively charged amino acids of the EPO receptor thereby improving the binding capacity.

It has been found that respective peptides which also carry a naphthylalanine in position 13 depict very good binding properties.

As mentioned, it is also possible to provide a negatively charged amino acid by converting a positively charged amino acid into a negatively charged amino acid. Thereby it is also possible to elongate the side chain thereby potentially enhancing the binding properties. According to this novel strategy, lysine (or homologous shorter amino acids like Dap, Dab or ornithine) is derivatized with a suitable agent providing negatively charged groups. A suitable agent is e.g. a diacid such as e.g. dicarboxylic acids or disulphonic acids. Glutaric acid, adipic acid, succinic acid, pimelic acid and suberic acid may be mentioned as examples.

According to a further aspect, the peptide according to the invention carries a positively charged amino acid in position X₁₀, X₁₇ and/or X₁₉. The positively charged amino acid is selected from the group consisting of
natural positively charged amino acids, e.g. lysine, arginine, histidine or ornithine;
non-natural positively charged amino-acids,
originally negatively charged amino acids which are, however, derivatized with suitable chemical groups in order to provide them with a positively charged group.

It turned out that very potent EPO mimetic peptides can be created when in position X₁₀ and/or X₁₇ of the peptide an amino acid is present which depicts an elongated side chain compared to lysine. This amino acid may be non-proteinogenic. According to one embodiment the elongation of the positively charged amino acid is provided by incorporating elongation units in the side chain of the amino acid to be elongated which does not necessarily need to be lysine. Also shorter amino acid may be used as starting materials which are then elongated by appropriate routine chemical reactions. Usually, the elongation units are either aliphatic (e.g. CH₂ units) or aromatic (e.g. phenyl or naphthyl units) groups. Examples of appropriate elongated amino acids are e.g. homoarginine, aminophenylalanine and aminonaphthylalanine.

According to a further embodiment of this first and second embodiment of the present invention, X₈ is a D-amino acid, preferably D-phenylalanine.

In case the consensus of the first and second embodiment also comprises an amino acid in position X₅, X₅ may be selected from any amino acid, however, it is preferably A, H, K, L, M, S, T or I.

In case X₄ is present in the peptide it may be selected from any amino acid, however, it is preferably F, Y or a derivative of F or Y, wherein the derivative of F or Y carries at least one electron-withdrawing substituent. The electron-withdrawing substituent is preferably selected from the group consisting of the amino group, the nitro group and halogens. Examples are 4-amino-phenylalanine, 3-amino-tyrosine, 3-iodo-tyrosine, 3-nitro-tyrosine, 3,5-dibromo-tyrosine, 3,5-dinitro-tyrosine, 3,5-diiodo-tyrosine.

In case X₃ is present in the consensus, X₃ is independently selected from any amino acid, preferably D, E, L, N, S, T or V.

Furthermore, especially in case the monomeric units (binding domains) are forming a dimer, it is preferred that the amino acids in the N-terminal region of the monomers (e.g. position X₁ and X₂) and the C-terminal region of the monomer (e.g. X₁₉ and X₂₀) depict a small flexible amino acid such as glycine or beta-alanine in order to provide a flexible conformation.

According to a third embodiment of the present invention a differently structured peptide is provided which also depicts good EPO mimetic properties. This peptide also comprises at least 10 amino acids, is capable of binding to the EPO receptor and comprises an agonist activity. The characteristics of this EPO mimetic peptide are described by at least one of the following core consensus sequences of amino acids:

X₉X₁₀X₁₁X₁₂X₁₃;
X₉X₁₀X₁₁X₁₂X₁₃X₁₄X₁₅X₁₆X₁₇
or
X₉X₁₀X₁₁X₁₂X₁₃X₁₄X₁₅X₁₆X₁₇X₁₈X₁₉

Each amino acid of these consensus sequences is selected from natural or non-natural amino acids. According to the essential feature of the second aspect of the present invention, at least one of the positions X₁₀, X₁₇ or X₁₉ depicts a negatively charged amino acid. Also comprised are peptides selected from the group consisting of functionally equivalent fragments, derivatives and variants of the above peptide consensus sequence, having EPO mimetic activity and having at least in one of the positions X₁₀, X₁₇ or X₁₉ a negatively charged amino acid.

It was very surprising that negatively charged amino acids in these positions depict such excellent EPO mimetic properties. The further amino acid positions (if present in the consensus) are defined as follows:

X₉ is G or a conservative exchange of G;
X₁₁ is selected from any amino acid;
X₁₂ is an uncharged polar amino acid or A; preferably threonine, serine, asparagine or glutamine;
X₁₃ is W, 1-nal, 2-nal, A or F;

### X₁₄ is D, E, I, L or V;

X₁₅ is an amino acid with a sidechain functionality capable of forming a covalent bond or A or α-amino-γ-bromobutyric acid,
X₁₆ is independently selected from any amino acid, preferably G, K, L, Q, R, S, Har or T;
X₁₈ is independently selected from any amino acid, preferably L or Q.

The peptides according to the third embodiment of the present invention carrying a negatively charged amino acid in at least one of the positions X₁₀, X₁₇ and/or X₁₉ (if present), are suitable candidates for a peptide depicting discriminating EPO mimetic properties in the human and the animal system. As pointed out above, the protein sequences of the EPO receptor of different species have only a few differences from species to species, and thus the EPO receptors are ranked as “highly conserved with negligible species differences”. However, it was surprisingly shown that EPO mimetic peptides with a negatively charged amino acid in at least one of the described positions may be able to discriminate between the peptide-binding sites of human and animal EPO-receptor. Peptides having a higher binding capacity to the animal receptor are preferably used for veterinary uses.

The peptide carrying a negatively charged amino acid in at least one of the positions X₁₀, X₁₇ and/or X₁₉ may comprise the following additional amino acids in the consensus:

X₆X₇X₈

wherein each amino acid is selected from natural or non-natural amino acids and wherein
X₆ is an amino acid with a sidechain functionality capable of forming a covalent bond or A or α-amino-γ-bromobutyric acid;

### X₇ is R, H, L, W or Y or S;

X₈ is M, F, I, Y, H, homoserinemethylether or norisoleucine.

Furthermore, the enlarged consensus may also be described by the following amino acids:

X₉X₁₀X₁₁X₁₂X₁₃X₁₄X₁₅X₁₆X₁₇X₁₈X₁₉

wherein each amino acid is selected from natural or non-natural amino acids and wherein
X₉ is G or a conservative exchange of G;
in case X₁₀ is not a negatively charged amino acid, X₁₀ is proline, a conservative exchange of proline or a non conservative exchange of proline or X₉ and X₁₀ are substituted by a single amino acid;
X₁₁ is selected from any amino acid;
X₁₂ is an uncharged polar amino acid or A; preferably threonine, serine, asparagine or glutamine;
X₁₃ is W, 1-nal, 2-nal, A or F;

### X₁₄ is D, E, I, L or V;

X₁₅ is an amino acid with a sidechain functionality capable of forming a covalent bond or A or α-amino-γ-bromobutyric acid;
X₁₆ is independently selected from any amino acid, preferably G, K, L, Q, R, S, Har or T;
in case X₁₇ is not a negatively charged amino acid, X₁₇ is selected from any amino acid, preferably A, G, P, Y or a positively charged natural, non-natural or derivatized amino acid, preferably K, R, H, ornithine or homoarginine;
X₁₈ is independently selected from any amino acid, preferably L or Q;
in case X₁₉ is not a negatively charged amino acid, X₁₉ is independently selected from any amino acid, preferably a positively charged amino acid such as K, R, H, ornithine or homoarginine or a small flexible amino acid such as glycine or beta-alanine;
provided that at least one of X₁₀, X₁₇ or X₁₉ is a negatively charged amino acid.

Of course, this embodiment of the invention also comprises peptides selected from the group consisting of functionally equivalent fragments, derivatives and variants of the above peptide consensus sequence, having EPO mimetic activity and having at least in one of the positions X₁₀, X₁₇ or X₁₉ a negatively charged amino acid.

It is preferred that the amino acids in positions X₆ and X₁₅ having a sidechain functionality allowing the formation of a covalent bond and hence the creation of a linking bridge within the peptide are chosen such that they are able to form a covalent bond with each other (please refer to the description of the first embodiment of the present invention above). Suitable amino acids are hence amino acids carrying SH-groups for forming disulfide bonds (e.g. cysteine and cysteine derivatives such as homocysteine) or thiolysine thereby only mentioning a few suitable candidates. Also selenide bridge forming amino acids such as selenocysteine are suitable. However, as described above, also other amino acids enabling the formation of a covalent bond e.g. an amide bond or a thioether bond are suitable. Hence a selection of preferred amino acids in position X₆ and X₁₅ comprises C, K, E, α-amino-γ-bromobutyric acid, homocysteine (hoc), and cysteine derivatives such as selenocysteine, thiolysine. This applies to all embodiments of the present invention.

Negatively charged amino acids present in the peptide according to the third embodiment of the present invention may be selected from the group consisting of
natural negatively charged amino acids, especially D or E;
non-natural negatively charged amino acids,
originally positively charged amino acids which are, however, derivatized with suitable chemical groups in order to provide them with a negatively charged group.

The non-natural negatively charged side chain may depict an elongated side chain. The elongated side chains are probably able to contact more efficiently the positively charged amino acids of the EPO receptor and thereby enhance the binding capacity. Examples for such amino acids are alpha-amino adipic acid (Aad), 2-aminoheptanediacid (2-aminopimelic acid) or alpha-aminosuberic acid (Asu).

As outlined, it is also possible to provide a negatively charged amino acid by converting a positively charged amino acid into a negatively charged amino acid. Thereby it is also possible to elongate the side chain. This may improve the binding properties to the EPO receptor. According to this novel strategy, a positively charged amino acid such as e.g. lysine (or homologous shorter amino acids like Dap, Dab or ornithine) is derivatized with a suitable agent providing negatively charged groups. A suitable agent is e.g. a diacid such as e.g. dicarboxylic acids or disulphonic acids. Glutaric acid, adipic acid, succinic acid, pimelic acid and suberic acid may be mentioned as examples.

A suitable example of a lysine, elongated and negatively charged with glutaric acid is provided below:

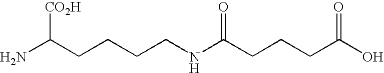

Another alternative for an elongating modification is a combination of lysine with adipic acid:

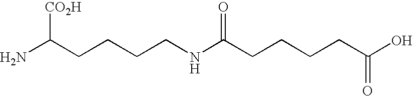

This elongation strategy which is very advantageous for improving the binding properties of the EPO mimetic peptides of the present invention may also be used for improving the characteristics of different molecules. It is thus a completely independent technological idea. Thus, also a modified amino acid is provided, wherein a positively charged amino acid is derivatized with suitable chemical groups in order to provide the positively charged amino acid with a negatively charged group. Thereby the originally positively charged amino acid is converted into a negatively charged amino acid. This is especially advantageous if the chemical modification also results in an elongation of the side chain which often improves the binding capacity. Suitable agents for modification are described above.

As outlined above, it is only necessary according to the second aspect of the present invention that one of the amino acid positions X₁₀, X₁₇ and/or X₁₉ is occupied by a negatively charged amino acid, even though also two or all positions may depict a respective amino acid. However, in case one or more of these positions are not occupied by a negatively charged amino acid, it is preferred that a positively charged amino acid is present in the other positions X₁₀, X₁₇ and/or X₁₉.

This positively charged amino acid is preferably selected from the group consisting of
natural positively charged amino acids, e.g. lysine, arginine, histidine and ornithine;
non-natural positively charged amino acids, such as e.g. homoarginine or diaminobutyric acid;
originally negatively charged amino acids which are, however, derivatized with suitable chemical groups in order to provide them with a positively charged group.

It turned out that very potent EPO mimetic peptides can be created when in position X₁₀ and/or X₁₇ a positively charged amino acid is present which depicts an elongated side chain compared to lysine. According to one embodiment the elongation of the positively charged amino acid is provided by incorporating elongation units in the side chain of an amino acid which does not necessarily need to be lysine. Also shorter amino acid may be used as starting materials which are then elongated by appropriate routine chemical reactions. Usually, the elongation units are either aliphatic (e.g. CH₂ units) or aromatic (e.g. phenyl or naphthyl units) groups. Examples of appropriate amino acids are e.g. homoarginine, aminophenylalanine and aminonaphthylalanine. Non-proteinogenic amino acids are preferred due to their greater variety. This embodiment combined with a negatively charged amino acid in at least one of the other amino acid positions X₁₀, X₁₇ and/or X₁₉ results in potent EPO mimetic peptides which are suitable candidates for a differentiating activity pattern in the human and animal model.

For EPO mimetic peptides for veterinary uses, it is preferred that a negatively charged amino acid is located in position 19. It was experimentally shown that peptides having the respective characteristic often depict a better binding capacity to animal EPO receptors.

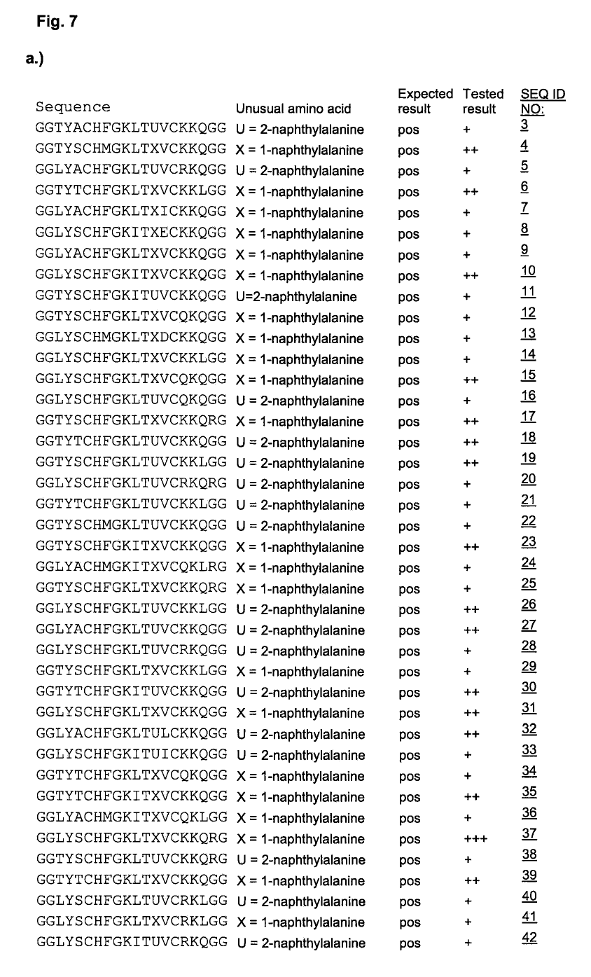

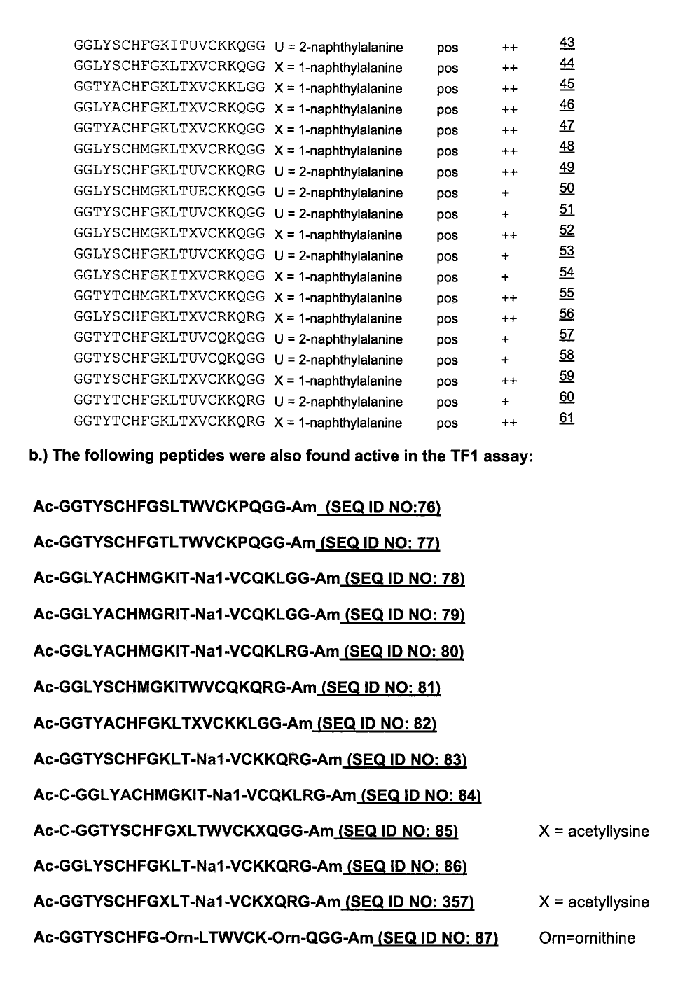

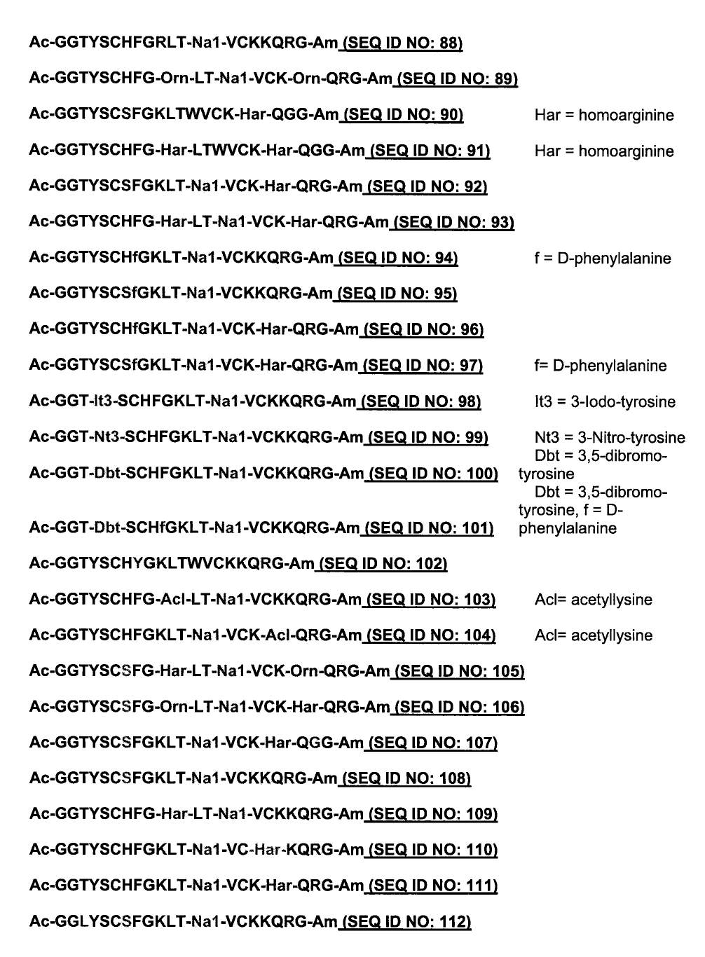

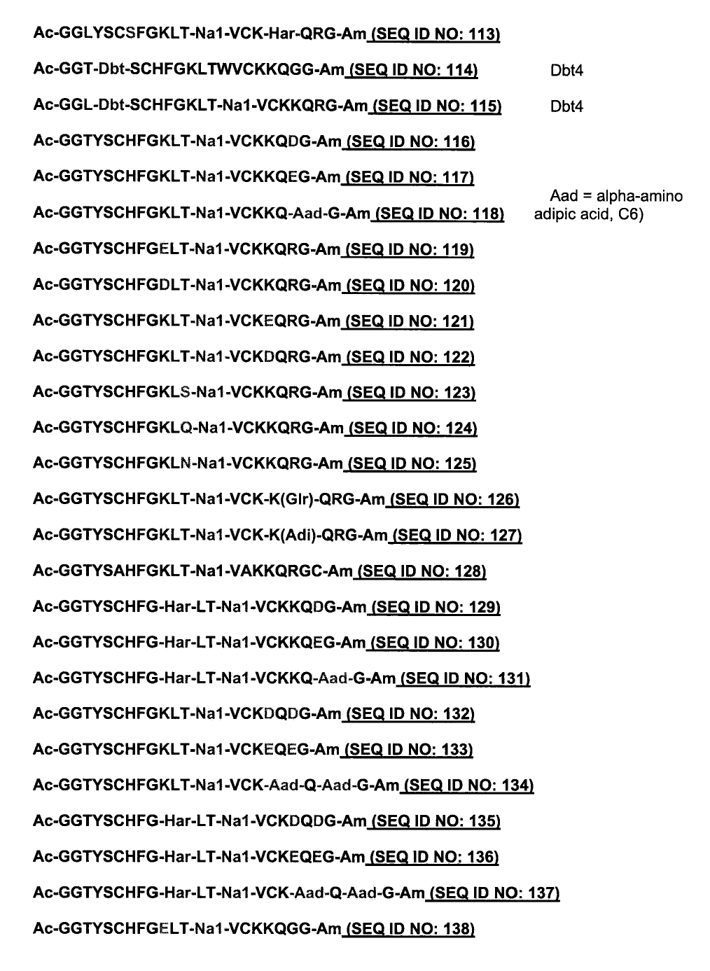

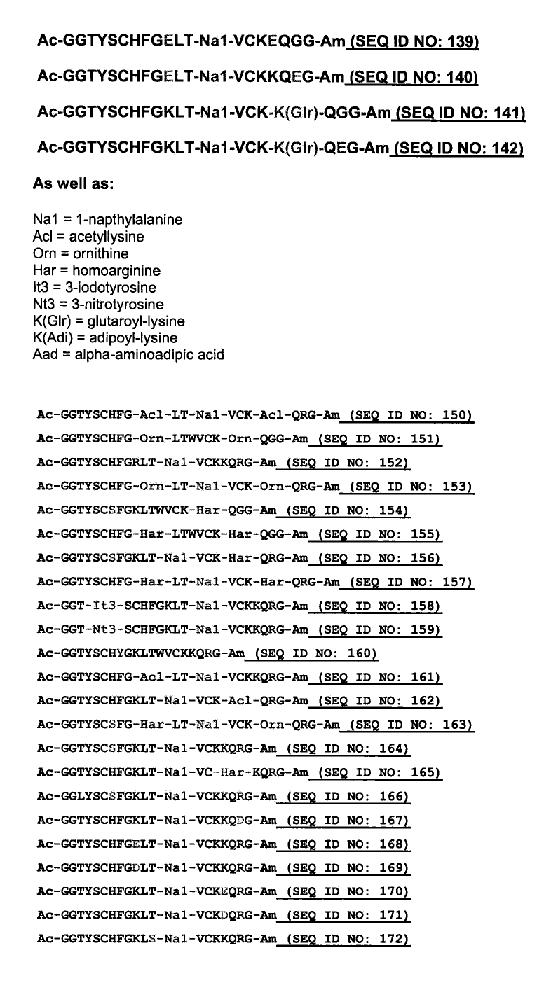

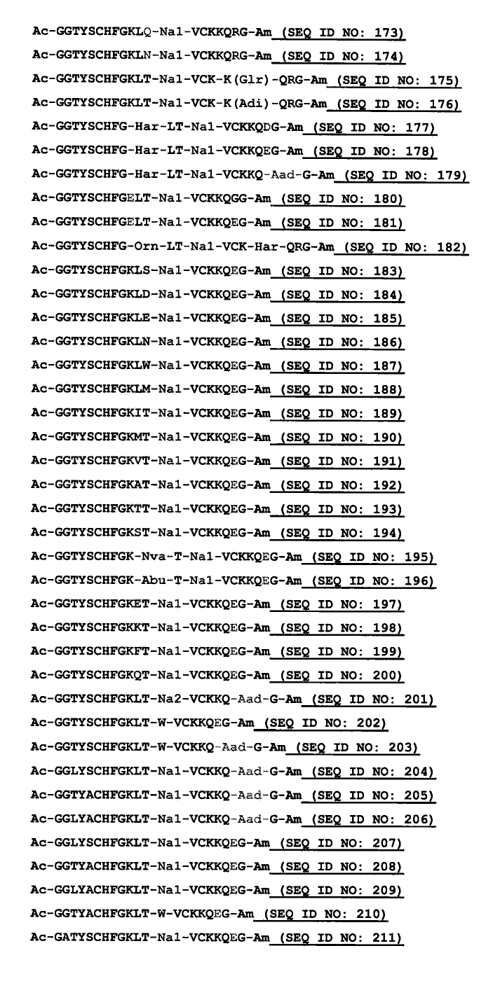

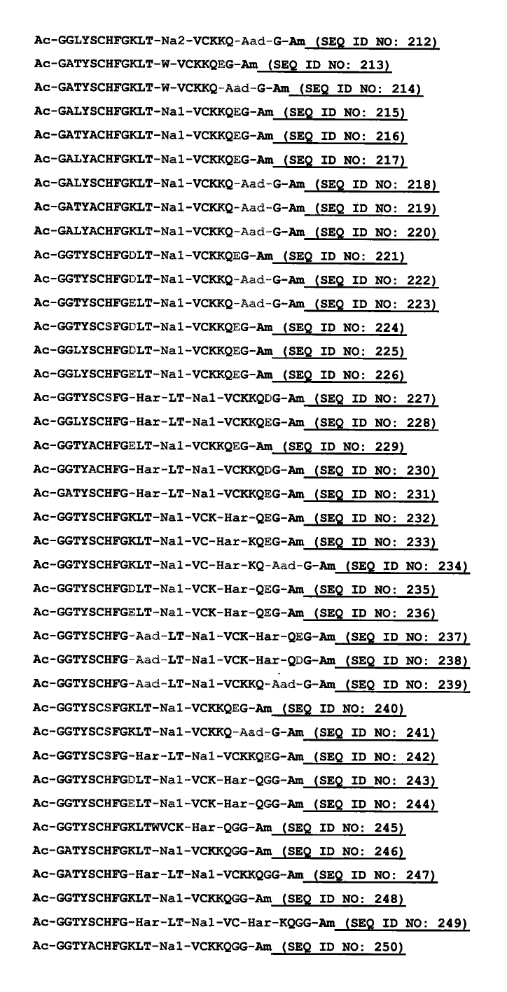

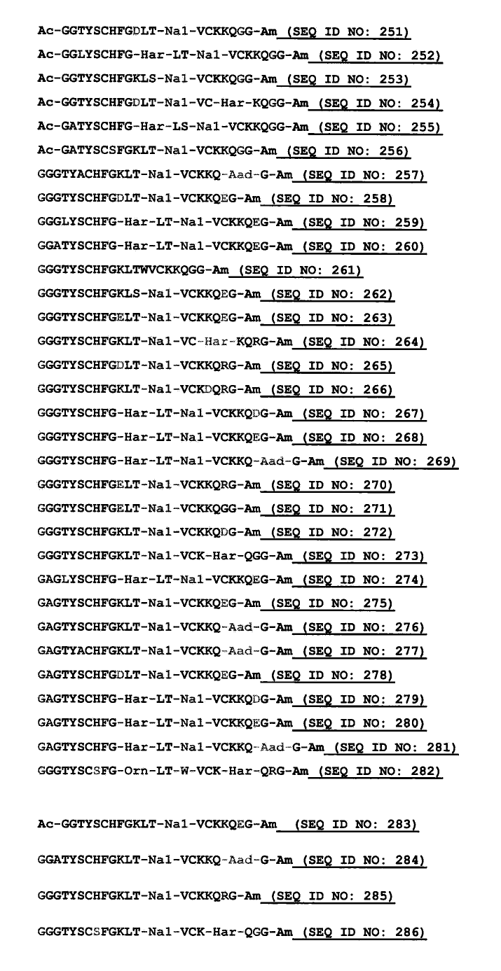

For veterinary uses, it is especially preferred, that the negatively charged amino acid in position 19 is selected from E, D or Aad. It is beneficial to combine this feature with a naphthylalanine (preferably NaI-1) in position 13. Furthermore, it is preferred that a positively charged amino acid is in position 17, preferably K or Har. It is also preferred that a positively charged amino acid is present in position 10, preferably lysine. Especially preferred examples of EPO mimetic peptides of this embodiment are shown in FIG. 7 c. In particular, an EPO mimetic peptide sequences for veterinary uses comprises an amino acid sequence which is selected from the group consisting of:

Ac-GGTYSCHFGKLT-Nal-VCK-Har-QDG-Am
Ac-GGTYSCHFGKLT-Nal-VCK-Har-Q-Aad-G-Am
GGGTYSCHFGKLT-Nal-VCKKQ-Aad-G-Am

This third embodiment of the present invention may also be combined with the feature wherein X₈ is a D-amino acid, preferably D-phenylalanine.

An enlarged consensus sequence of this embodiment comprises the following additional amino acids:

X₄ may be F, Y or a derivative of F or Y, wherein the derivative of F or Y carries at least one electron-withdrawing substituent. As already described above in conjunction with the second embodiment, the electron-withdrawing substituent is preferably selected from the group consisting of the amino group, the nitro group and halogens. Suitable examples are 4-amino-phenylalanine, 3-amino-tyrosine, 3-iodo-tyrosine, 3-nitro-tyrosine, 3,5-dibromo-tyrosine, 3,5-dinitro-tyrosine, 3,5-diiodo-tyrosine.

X₅ may be selected from any amino acid, however, it is preferably A, H, K, L, M, S, T or I.

Also X₃ may be present and my be independently selected from any amino acid, preferably D, E, L, N, S, T or V.

Furthermore, especially in case the monomeric units are forming a dimer it is preferred that the amino acids in the beginning of the monomers (e.g. position X₁ and X₂) and the end of the monomer (e.g. X₁₉ and X₂₀) depict small flexible amino acids such as glycine or beta-alanine in order to provide a flexible conformation.

As already described in conjunction with the second embodiment of the present invention, it is advantageous to provide a naphthylalanine (nal-1 or nal-2) in position X₁₃. The incorporation of NaI in position 13 leads to stronger hydrophobic interactions between the peptide monomers as described above thereby potentially enhancing the dimerisation of the monomeric peptide chains and possibly stabilising the conformation of the peptide dimer thereby improving the EPO mimetic activity. A combination of both embodiments (second and third) is very favourable.

Examples of suitable peptide sequences comprising naphthylalanine are provided in FIG. 7a.

According to a fourth embodiment of the present invention a peptide of at least 10 amino acids in length is provided, capable of binding to the EPO receptor and comprising an agonist activity, comprising the following core sequence of amino acids:

X₈X₉X₁₀X₁₁X₁₂X₁₃X₁₄X₁₅

wherein each amino acid is selected from natural or non-natural amino acids and wherein
X₈ is a D-amino acid;
X₉ is G or a conservative exchange of G;
X₁₀ is proline, a conservative exchange of proline or a non conservative exchange of proline;
or X₉ and X₁₀ are substituted by a single amino acid;
X₁₁ is selected from any amino acid;
X₁₂ is an uncharged polar amino acid or A; preferably threonine, serine, asparagine or glutamine;
X₁₄ is D, E, I, L or V;
X₁₅ is an amino acid with a sidechain functionality capable of forming a covalent bond or A or α-amino-γ-bromobutyric acid.

Also comprised are peptides selected from the group consisting of functionally equivalent fragments, derivatives and variants of the peptide consensus sequence according to the fourth embodiment, having EPO mimetic activity and having a D-amino acid in position 8.

The prominent feature of the fourth embodiment of the present invention is the presence of a D-amino acid in position X₈. D-phenylalanine is preferred. This embodiment appears to be a good candidate for differentiating between the animal and human EPO-Receptor. The inversion of the alpha-C-atom in position 8 leads to a different geometrical position of the phenyl group, which could better fit with the animal receptor, especially the canine EPOR.

This fourth aspect of the present invention may also be combined with the further advantageous embodiments as is described subsequently.

The peptide according to the fourth embodiment of the invention may also be described by the following enlarged amino acid core sequence

X₆X₇X₈X₉X₁₀X₁₁X₁₂X₁₃X₁₄X₁₅

wherein each amino acid is selected from natural or non-natural amino acids and wherein
X₆ is an amino acid with a sidechain functionality capable of forming a covalent bond or A or α-amino-γ-bromobutyric acid;
X₇ is R, H, L, W or Y or S;
X₈ is D-M, D-F, D-1, D-Y, D-H, D-homoserinemethylether or D-norisoleucine;
X₉ is G or a conservative exchange of G;
X₁₀ is proline, a conservative exchange of proline or a non conservative exchange of proline;
or X₉ and X₁₀ are substituted by a single amino acid;
X₁₁ is selected from any amino acid;
X₁₂ is an uncharged polar amino acid or A; preferably threonine, serine, asparagine or glutamine;
X₁₄ is D, E, I, L or V;
X₁₅ is an amino acid with a sidechain functionality capable of forming a covalent bond or A or α-amino-γ-bromobutyric acid.

A further embodiment of the fourth embodiment of the present invention may be described by the following amino acid sequence:

X₆X₇X₈X₉X₁₀X₁₁X₁₂X₁₃X₁₄X₁₅X₁₆X₁₇X₁₈X₁₉

wherein X₆-X₁₅ have the above meaning as described in conjunction with the fourth embodiment of the invention and wherein
X₁₆ is independently selected from any amino acid, preferably G, K, L, Q, R, S, Har or T;
X₁₇ is independently selected from any amino acid, e.g. A, G, P, Y or a charged natural, non-natural or derivatized amino acid, preferably K, R, H, ornithine or homoarginine in case of a positively charged amino acid;
X₁₈ is independently selected from any amino acid, preferably L or Q;
X₁₉ is independently selected from any amino acid.

Also in conjunction with the fourth embodiment of the present invention, it is preferred that a charged amino acid is present in position X₁₀, X₁₇ and/or X₁₉. Experiments showed that very good EPO mimetic activity rates are achieved with charged amino acids. However, in general also uncharged but polar amino acids (such as e.g. serine, threonine, asparagine or glutamine) in these positions provide good results, if combined with the right amino acids in the other positions.

The charged amino acid in position X₁₀, X₁₇ and/or X₁₉ is either positively or negatively charged and is selected from the group consisting of natural amino acids, non-natural amino acids and derivatized amino acids.

According to one aspect, X₁₀, X₁₇ and/or X₁₉ is a negatively charged amino acid. Said negatively charged amino acid is preferably selected from the group consisting of
natural negatively charged amino acids, especially D or E;
non-natural negatively charged amino acids,
originally positively charged amino acids which are, however, derivatized with suitable chemical groups in order to provide them with a negatively charged group.

The non-natural negatively charged side chain may depict an elongated side chain. Examples for such amino acids are alpha-amino adipic acid (Aad), 2-aminoheptanediacid (2-aminopimelic acid) or alpha-aminosuberic acid (see above).

As outlined, it is also possible to provide a negatively charged amino acid by converting a positively charged amino acid into a negatively charged amino acid. Thereby it is also possible to elongate the side chain thereby enhancing the binding properties. According to this novel strategy (see above for details), lysine (or homologous shorter amino acids like Dap, Dab or ornithine) is derivatized with a suitable agent providing negatively charged groups. A suitable agent is e.g. a diacid such as e.g. dicarboxylic acids or disulphonic acids. Glutaric acid, adipic acid, succinic acid, pimelic acid and suberic acid may be mentioned as examples.

According to a further aspect, the peptide carries a positively charged amino acid in position X₁₀, X₁₇ and/or X₁₉. The positively charged amino acid is selected from the group consisting of
natural positively charged amino acids, e.g. lysine, arginine, histidine or ornithine;
non-natural positively charged amino acids,
originally negatively charged amino acids which are, however, derivatized with suitable chemical groups in order to provide them with a positively charged group.

It turned out that very potent EPO mimetic peptides can be created when in position X₁₀ and/or X₁₇ an amino acid is present which depicts an elongated side chain compared to lysine. According to one embodiment the elongation of the positively charged amino acid is provided by incorporating elongation units in the side chain of an amino acid which does not necessarily need to be lysine. Also shorter amino acids may be used as starting materials which are then elongated by appropriate routine chemical reactions (see above). Usually, the elongation units are either aliphatic (e.g. CH₂ units) or aromatic (e.g. phenyl or naphthyl units) groups. Examples of appropriate amino acids are e.g. homoarginine, aminophenylalanine and aminonaphthylalanine. Non-proteinogenic amino acids are preferred due to the greater variety. An alternative way is the derivatisation of amino acids with positively charged groups which not only allow a charge reversion (to a positive charge) but also provide an easy way for elongation of the molecule.

According to a further development of this embodiment the peptide is defined by the following enlarged amino acid core sequence:

X₄X₅X₆X₇X₈X₉X₁₀X₁₁X₁₂X₁₃X₁₄X₁₅X₁₆X₁₇X₁₈X₁₉

wherein X₆ to X₁₉ have the above meaning as described in conjunction with the fourth aspect of the present invention and wherein
X₄=is F, Y or a derivative of F or Y, wherein the derivative of F or Y carries at least one electron-withdrawing substituent;
X₅=is selected from any amino acid, preferably A, H, K, L, M, S, T or I.

The electron-withdrawing substituent is preferably selected from the group consisting of the amino group, the nitro group and halogens. X₄ may also be selected from the group consisting of 4-amino-phenylalanine, 3-amino-tyrosine, 3-iodo-tyrosine, 3-nitro-tyrosine, 3,5-dibromo-tyrosine, 3,5-dinitro-tyrosine, 3,5-diiodo-tyrosine.

Also X₃ may be present and may be independently selected from any amino acid, preferably D, E, L, N, S, T or V.

Furthermore, in case the monomeric units are forming a dimer it is preferred that the amino acid positions in the beginning of the monomers (e.g. position X₁ and X₂) and the end of the monomer (e.g. X₁₉ and X₂₀) depict a small flexible amino acid such as glycine or beta-alanine in order to provide conformational flexibility.

As already described in conjunction with the second embodiment of the present invention, it is advantageous to provide a naphthylalanine in position X₁₃. The incorporation of NaI in position 13 leads to stronger hydrophobic interactions between the peptide monomers as described above thereby potentially enhancing the dimerisation of the monomeric peptide chains and possibly stabilising the conformation of the peptide dimer thereby improving the EPO mimetic activity.

According to a fifth embodiment of the present invention, a peptide is provided which is also a good candidate for an EPO mimetic peptide depicting a species discriminating activity. This peptide comprises at least 10 amino acids, is capable of binding to the EPO receptor and comprises an agonist activity. This EPO mimetic peptide comprises the following core sequence of amino acids:

X₄X₅X₆X₇X₈X₉X₁₀X₁₁X₁₂X₁₃X₁₄X₁₅

wherein each amino acid is selected from natural or non-natural amino acids and wherein
X₄=is F, or a derivative of either F or Y, wherein the derivative of F or Y carries at least one electron-withdrawing substituent;
X₅=is selected from any amino acid, preferably A, H, K, L, M, S, T or I.
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
X₁₀ is non-conservative exchange of proline or X₉ and X₁₀ are substituted by a single amino acid;
X₁₁ is selected from any amino acid;
X₁₂ is an uncharged polar amino acid or A; preferably threonine, serine, asparagine or glutamine;
X₁₄ is D, E, I, L or V;
X₁₅ is an amino acid with a sidechain functionality capable of forming a covalent bond or A or α-amino-γ-bromobutyric acid.

Also comprised are peptides selected from the group consisting of functionally equivalent fragments, derivatives and variants of the above peptide consensus sequence, having EPO mimetic activity and having an amino acid in position X₄ which is selected from F, or a derivative of either F or Y, wherein the derivative of F or Y carries at least one electron-withdrawing substituent.

The electron-withdrawing substituent may be selected from the group consisting of the amino group, the nitro group and halogens. X₄ is preferably selected from the group consisting of 4-amino-phenylalanine, 3-amino-tyrosine, 3-iodo-tyrosine, 3-nitro-tyrosine, 3,5-dibromo-tyrosine, 3,5-dinitro-tyrosine, 3,5-diiodo-tyrosine.

Further advantageous combinations of this fifth embodiment of the invention with further embodiments are described in the dependent claims. For details about the respective features, please also refer to the description above, explaining the features in conjunction with the respective embodiments in detail. Combinations of the X₄ mutation and the D-phenylalanine mutation are especially suitable.

According to a further embodiment of the present invention, several alternative peptides are provided for providing improved EPO mimetic peptides. According to this sixth embodiment of the invention a peptide of at least 10 amino acids in length is provided, which is capable of binding to the EPO receptor and comprises an agonist activity.

Alternative (a) of this sixth embodiment comprises at least one of the following core sequences of amino acids:

X₉X₁₀X₁₁X₁₂X₁₃;
X₉X₁₀X₁₁X₁₂X₁₃X₁₄X₁₅X₁₆X₁₇
or
X₉X₁₀X₁₁X₁₂X₁₃X₁₄X₁₅X₁₆X₁₇X₁₈X₁₉

wherein each amino acid is selected from natural or non-natural amino acids, and wherein:
X₉ is G or a conservative exchange of G;
X₁₁ is selected from any amino acid;
X₁₂ is an uncharged polar amino acid or A; preferably threonine, serine, asparagine or glutamine;
X₁₃ is W, naphthylalanine, A or F;
X₁₄ is D, E, I, L or V;
X₁₅ is an amino acid with a sidechain functionality capable of forming a covalent bond or A or α-amino-γ-bromobutyric acid;
wherein at least one of the positions X₁₀, X₁₆, X₁₇ or X₁₉ depicts a positively charged non-proteinogenic amino acid having a side chain which is elongated compared to lysine.

Also comprised are peptides selected from the group consisting of functionally equivalent fragments, derivatives and variants of the above peptide consensus sequence having EPO mimetic activity and having an amino acid in at least one of the positions X₁₀, X₁₆, X₁₇ or X₁₉ depicts a positively charged non-proteinogenic amino acid having a side chain which is elongated compared to lysine.

This sixth embodiment of the invention describes an alternative strategy which also opens the option to potentially discriminate between the human and animal receptor by elongating positively charged side chains in the EPO mimetic peptides in at least one of the positions X₁₀, X₁₆, X₁₇ and/or X₁₉. This embodiment provides suitable candidates for a discriminating peptide since there are fewer negatively charged docking points in the murine and canine EPO receptor, and these docking points are harder reachable with shorter positively side chains (e.g. lysine). Thus, the incorporation of positively charged residues with a longer sidechain has a high potential to increase the affinity of the peptides to the EPO receptors.

Sequences which depict a homoarginine in position X₁₀ and/or X₁₇ were already disclosed in applicant's earlier application PCT/EP 2005/012075. According to the patent law of some countries this disclosure might constitute prior art.

Where this is applicable and could question patentability of the above consensus, the consensus sequence of the first alternative of the sixth embodiment of the invention for legal reasons may not comprise sequences disclosed in PCT/EP 2005/012075. This could apply to the consensus sequences selected from the following group:
a peptide, especially one being capable of binding the EPO receptor comprising the following sequence of amino acids:

X₆X₇X₈X₉X₁₀X₁₁X₁₂X₁₃X₁₄X₁₅

wherein each amino acid is selected from natural or unnatural amino acids and
X₆ is C, A, E, α-amino-V-bromobutyric acid or homocysteine (hoc);
X₇ is R, H, L, W or Y or S;
X₈ is M, F, I, homoserinemethylether or norisoleucine;
X₉ is G or a conservative exchange of G;
X₁₀ is Har
X₁₁ is selected from any amino acid;
X₁₂ is T or A;
X₁₃ is W, 1-nal, 2-nal, A or F;
X₁₄ is D, E, I, L or V;
X₁₅ is C, A, K, α-amino-γ-bromobutyric acid or homocysteine (hoc) provided that either X₆ or X₁₅ is C or hoc or
   a peptide, comprising the following amino acid sequence

X₃X₄X₅X₆X₇X₈X₉X₁₀X₁₁X₁₂X₁₃X₁₄X₁₅X₁₆X₁₇X₁₈

wherein X₆ to X₁₅ have the above meaning and wherein
X₃ is independently selected from any amino acid, preferably D, E, L, N, S, T or V;
X₄ is Y;
X₅ is independently selected from any amino acid, preferably A, H, K, L, M, S, T or I.
X₁₆ is independently selected from any amino acid, preferably G, K, L, Q, R, S or T;
X₁₇ is homoarginine;
X₁₈ is independently selected from any amino acid. or

GGTYSCSFGKLTWVCK-Har-QGG
GGTYSCHFG-Har-LTWVCK-Har-QGG

These sequences were already described in applicant's earlier PCT application PCT EP_{—}2005-01 20 75.

In countries where the postpublished disclosure of PCT/EP2005/012075 does not constitute a patentability problem, the above listed consensus and peptide sequences need not to be disclaimed from the broad consensus of the first alternative of the sixth embodiment.

According to a further development of the sixth embodiment of the present invention, the peptide comprises the following enlarged core sequence of amino acids:

X₆X₇X₈X₉X₁₀X₁₁X₁₂X₁₃X₁₄X₁₅X₁₆X₁₇X₁₈X₁₉

wherein each amino acid is selected from natural or non-natural amino acids and wherein
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
in case X₁₀ is not a positively charged non-proteinogenic amino acid having a side chain which is elongated compared to lysine, X₁₀ is proline, a conservative exchange of proline or a non conservative exchange of proline or X₉ and X₁₀ are substituted by a single amino acid;
X₁₁ is selected from any amino acid;
X₁₂ is an uncharged polar amino acid or A; preferably threonine, serine, asparagine or glutamine;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid;
in case X₁₆ is not a positively charged non-proteinogenic amino acid having a side chain which is elongated compared to lysine, X₁₆ is independently selected from any amino acid, preferably G, K, L, Q, R, S or T;
in case X₁₇ is not a positively charged non-proteinogenic amino acid having a side chain which is elongated compared to lysine, X₁₇ is selected from any amino acid, preferably A, G, P, Y or a positively charged natural, non-natural or derivatized amino acid, preferably K, R, H or ornithine;
X₁₈ is independently selected from any amino acid, preferably L or Q;
in case X₁₉ is not a positively charged non-proteinogenic amino acid having a side chain which is elongated compared to lysine, X₁₉ is independently selected from any amino acid, preferably a charged amino acid such as positively charged amino acid such as K, R, H or ornithine or negatively charged amino acid such as D, E or Aad;
   provided that at least one of X₁₀, X₁₆, X₁₇ or X₁₉ is a positively charged non-proteinogenic amino acid having a side chain which is elongated compared to lysine.

According to a further embodiment, at least one of X₁₀, X₁₆, X₁₇ or X₁₉ is a positively charged amino acid and wherein the positively charged amino acid is preferably selected from the group consisting of:
natural positively charged amino acids, e.g. lysine, arginine, histidine and ornithine;
non-natural positively charged amino acids,
originally negatively charged amino acids which are, however, derivatized with suitable chemical groups in order to provide them with a positively charged group;
   provided that at least one of X₁₀, X₁₆, X₁₇ or X₁₉ is a positively charged non-proteinogenic amino acid having a side chain which is elongated compared to lysine.

As described above, the elongation of the positively charged amino acid may be provided by elongation units of the side chain, wherein the elongation units are either aliphatic or aromatic groups. The elongation can be e.g. be provided by CH₂ units, wherein the number of CH₂ units is preferably between 1 and 6. Alternatively, the elongation can also be achieved with aromatic groups such as e.g. phenyl or naphthyl units.

The positively charged non-proteinogenic amino acid which is elongated compared to lysine, is preferably a non-natural amino acid. Non-natural amino acids offer more choices thereby alleviating the possibility to find a perfectly fitting elongated amino acid. Examples for suitable non-natural elongated amino acids are e.g. homoarginine, aminophenylalanine and aminonaphthylalanine.

An elongated positively charged side chain in position X₁₇ seems to interact better with the murine/canine EPO receptor. Especially homoarginine, which is an artificial elongated homologous arginine, proved to be suitable. This amino acid is outreaching lysine and is able to interact with more distant negatively charged amino acids in the murine/canine EPO receptors (Glu60 and Glu62 in the animal EPO receptors).

An elongated positively charged side chain in position X₁₀ has a similar effect as the mutation in position X₁₇ described above. Also in this case, more distant negatively charged amino acids might be reached through the elongation (Glu34 in the murine/canine EPO receptor).

It is desirable to combine the mutations/features in positions X₁₀ and X₁₇. The geometry of a peptide carrying an elongated positively charged amino acid (e.g. homoarginine) in both positions indicates a strong interaction with the EPO receptor. As described, the amino acid is preferably non-proteinogenic. The strength of the provided electrostatic interaction is even intensified by the multiple hydrogen bonds from each homoarginine residue.

According to the sixth embodiment of the invention at least one of X₁₀, X₁₆, X₁₇ and/or X₁₉ depicts a non-proteinogenic elongated positively charged amino acid. The other positions of X₁₀, X₁₆, X₁₇ and/or X₁₉ may also depict a charged amino acid, which is either positively or negatively charged and is selected from the group consisting of natural amino acids, non-natural amino acids and derivatised amino acids.

According to one alternative at least one of X₁₀, X₁₇ and/or X₁₉ is a negatively charged amino acid.

In case X₁₀, X₁₇ and/or X₁₉ is a negatively charged amino acid, said negatively charged amino acid is preferably selected from the group consisting of
natural negatively charged amino acids, especially D or E;
non-natural negatively charged amino acids,
originally positively charged amino acids which are, however, derivatized with suitable chemical groups in order to provide them with a negatively charged group.

The non-natural negatively charged side chain may depict an elongated side chain. Examples for such amino acids are alpha-amino adipic acid (Aad), 2-aminoheptanediacid (2-aminopimelic acid) or alpha-aminosuberic acid.

As outlined, it is also possible to provide a negatively charged amino acid by converting a positively charged amino acids into a negatively charged amino acid. Thereby it is also possible to elongate the side chain thereby enhancing the binding properties. According to this novel strategy, lysine (or homologous shorter amino acids like Dap, Dab or ornithine) is derivatized with a suitable agent providing negatively charged groups. A suitable agent is e.g. a diacid such as e.g. dicarboxylic acids or disulphonic acids. Glutaric acid, adipic acid, succinic acid, pimelic acid and suberic acid may be mentioned as examples. Please also refer to our above detailed discussion of this embodiment.

Under the provision that at least one of the positions X₁₀, X₁₆, X₁₇ and/or X₁₉ depicts an elongated positively charged non-proteinogenic amino acid the peptide may also carry a “normal” positively charged amino acid in position X₁₀, X₁₆, X₁₇ and/or X₁₉. The positively charged amino acid is selected from the group consisting of
natural positively charged amino acids, e.g. lysine, arginine, histidine or ornithine;
non-natural positively charged amino acids,
originally negatively charged amino acids which are, however, derivatized with suitable chemical groups in order to provide them with a positively charged group.

A further development of the sixth embodiment of the present invention provides in X₈ a D-amino acid, preferably D-phenylalanine.

According to a further development of the sixth embodiment, the peptide comprises the following enlarged amino acid core sequence:

X₄X₅X₆X₇X₈X₉X₁₀X₁₁X₁₂X₁₃X₁₄X₁₅X₁₆X₁₇X₁₈X₁₉

wherein X₆ to X₁₉ have the above meaning and wherein
X₄=is F, Y or a derivative of F or Y, wherein the derivative of F or Y carries at least one electron-withdrawing substituent;
X₅=is selected from any amino acid, preferably A, H, K, L, M, S, T or I.

As described above, the electron-withdrawing substituent may be selected from the group consisting of the amino group, the nitro group and halogens. Examples are 4-amino-phenylalanine, 3-amino-tyrosine, 3-iodo-tyrosine, 3-nitro-tyrosine, 3,5-dibromo-tyrosine, 3,5-dinitro-tyrosine, 3,5-diiodo-tyrosine.

Also X₃ may be present and may be independently selected from any amino acid, preferably D, E, L, N, S, T or V.

Furthermore, in case the monomeric units are forming a dimer via a continuous peptide linker, it is preferred that the amino acids in the N-terminal region of the monomers (e.g. position X₁ and X₂) and the C-terminal region of the monomer (e.g. X₁₉ and X₂₀) depict a small flexible amino acid such as glycine or beta-alanine in order to provide a flexible conformational.

The application describes besides EPO mimetic peptides in general different means and strategies in order to improve the EPO mimetic activity and/or in order to allow a discrimination between human and animal EPO-R. As described, the different strategies and aspects of the invention can be combined with each other in order to achieve a “tailored” EPO mimetic peptide depicting the desired properties. It is thus important to understand that the described strategies can be understood as design units, which can be independently combined with each other in order to come to an EPO mimetic peptide having the desired properties. E.g. the characteristics of the second embodiment (naphthylalanine in position X₁₃) may combined with the characteristics of the third embodiment, that at least one of X₁₀, X₁₇ and X₁₉ are negatively charged.

The length of the peptides according to the embodiments one to six described above is preferably between ten to forty or fifty or sixty amino acids. In preferred embodiments, the peptide consensus depicts a length of at least 10, 15, 18, 20 or 25 amino acids. Of course, the described consensus sequences may be embedded respectively be comprised by longer sequences. The described peptide consensus sequences can be perceived as forming binding domains for the EPO receptor. As described above and below, it is also possible to combine the monomeric peptide units (binding domains) to peptide di- or even multimers. In case a peptide linker is used for creating the di- or multimer also longer peptides are created due to dimerisation and/or multimerisation. As EPO mimetic peptides they are capable of binding to the EPO receptor.

The EPO mimetic peptide sequences according to the invention can have N-terminal and/or C-terminal acetylations and amidations. Some amino acids may also be phosphorylated.

The peptides according to the invention may comprise besides L-amino acids or the stereoisomeric D-amino acids, unnatural/unconventional amino acids, such as e.g. alpha, alpha-disubstituted amino acids, N-alkyl amino acids or lactic acid, e.g. 1-naphthylalanine, 2-naphthylalanine, homoserine-methylether, 9-alanine, 3-pyridylalanine, 4-hydroxyproline, O-phosphoserine, N-methylglycine (sarcosine), homoarginine, N-acetylserine, N-acetylglycine, N-formylmethionine, 3-methylhistidine, 5-hydroxylysine, nor-lysine, 5-aminolevulinic acid or aminovaleric acid. The use of N-methylglycine (MeG) and N-acetylglycine (AcG) is especially preferred, in particular in a terminal position. Also within the scope of the present invention are peptides which are retro, inverso and retro/inverso peptides of the defined peptides and those peptides consisting entirely of D-amino acids.

The present invention also relates to the derivatives of the peptides, e.g. oxidation products of methionine, or deamidated glutamine, arginine and C-terminus amide.

According to one development of the embodiments of the invention the peptides do have a single amino acid substituting the amino acid residues X₉ and X₁₀. In this embodiment also both residues may be substituted by one non-natural amino acid, e.g. 5-aminolevulinic acid or aminovaleric acid.

According to a further development, the peptides described in the first to sixth embodiment comprise in X₆ and/or X₁₅ as an amino acid with an bridge forming functionality C, a cysteine derivative such as selenocysteine, E, K, or hoc, and/or X₇ as R, H or Y or S and/or X₈ as F or M and/or X₉ as G or A, preferably G and/or X₁₀ as K or Har and/or X₁₁ as V, L, I, M, E, A, T or norisoleucine and/or X₁₂ as T and/or X₁₃ as W or naphthylalanine and/or X₁₄ as D or V and/or X₁₇ as P, Y or A or a basic natural or non-natural amino acid. It is, however, also preferred as described above that X₁₇ is K or a non-natural amino acid with a positively charged side chain such as e.g. homoarginine.

Fragments, derivatives and variant polypeptides according to the present invention retain substantially the same biological function or activity as the peptides according to the individual embodiments described herein. In order to discriminate them properly from the state of the art the fragment, derivatives or variants have the same characteristic features as the respective embodiments:
regarding embodiment 1 they have an amino acid in position X₁₀ that constitutes a non-conservative exchange of proline or wherein X₉ and X₁₀ are substituted by a single amino acid;
regarding embodiment 2 they have an amino acid in position X₁₀ that constitutes a non-conservative exchange of proline or wherein X₉ and X₁₀ are substituted by a single amino acid and a naphthylalanine in position X₁₃;
regarding embodiment 3, at least one of the positions X₁₀, X₁₇ or X₁₉ is a negatively charged amino acid;
regarding embodiment 4, they carry a D-amino acid in position X₈;
regarding embodiment 5, they have an amino acid in position X₁₀ that constitutes a non-conservative exchange of proline or wherein X₉ and X₁₀ are substituted by a single amino acid and have in position X₄ F, or a derivative of either F or Y, wherein the derivative of F or Y carries at least one electron-withdrawing substituent;
regarding embodiment 6, at least one of the positions X₁₀, X₁₆, X₁₇ or X₁₉ depicts a positively charged non-proteinogenic amino acid having a side chain which is elongated compared to lysine.

“A fragment” is less than a full length peptide (or polypeptide, the term peptide as used herein does not comprise any size restrictions), which retains substantially similar functional activity.

“Derivatives” include peptides that have been chemically modified to provide an additional structure and/or function.

Derivatives can be modified by either natural processes or by chemical modification techniques, both of which are well known in the art. Modifications can occur anywhere in a polypeptide, including the peptide backbone, the amino acid side-chains, and the amino or carboxyl termini.

Other chemical modifications include e.g. acetylations, acylation, amidation, covalent attachment of different chemical moieties, cross-linking, cyclization, disulfide bond or other bridge formations, hydroxylation, methylation, oxidation, PEGylation, selenoylation.

“Variants” of peptides according to the present invention include polypeptides having one or more amino acid sequence exchanges with respect to the amino acids defined in the consensus. Of course, the may also contain amino acids other than natural amino acids.

E.g., one or more conservative amino acid substitutions can be carried out within the amino acid sequence of the polypeptides according to this invention in order to arrive at functional variants of the different embodiments of the invention as described above. The substitution occurs e.g. within amino acids having unpolar side chains, the natural or non-natural uncharged D- or L amino acids with polar side chains, amino acids with aromatic side chains, the natural or non-natural positively charged D- or L-amino acids, the natural or non-natural negatively charged D- or L amino acids as well as within any amino acids of similar size and molecular weight, wherein the molecular weight of the original amino acid should not deviate more than approximately +/−25% of the molecular weight of the original amino acid and the binding capacity to the receptor of the hormone erythropoietin with agonistic effect is maintained. Preferably, no more than 1, 2 or 3 amino acids are substituted. Sequence variants wherein no proline is introduced at the positions 10 and 17 are preferred.

The peptide sequences described herein can be used as suitable monomeric peptide units which constitute binding domains for the EPO receptor. They can be used in their monomeric form since they bind to the EPO receptor. As described herein, they are preferably used as dimers since it was shown that the capacity to induce dimerisation of the EPO receptor and thus biological activity is enhanced by dimerisation of the monomeric binding units.

Thus it is clear that many different peptides are within the scope of the present invention. It has been found however, that the sequence Ac-VLPLYRCRMGRETWECMRAAGVTK-NH₂ has certain disadvantages and is thus not preferred according to the present invention.

At the beginning (N terminal) and end (C terminal) of the described individual peptide sequences, up to five amino acids may be removed and/or added. It is self-evident that size is not of relevance as long as the peptide function is preserved. Furthermore, please note that individual peptide sequences that might be too short to enfold their activity as monomers usually function as agonists upon dimerisation. Such peptides are thus preferably used in their dimeric form. Respective truncated and or elongated embodiments are thus also comprised by the spirit of the invention.

In the present invention, the abbreviations for the one-letter code as capital letters are those of the standard polypeptide nomenclature, extended by the addition of non-natural amino acids.

| Code | Amino acid |
|---|---|
| A | L-alanine |
| V | L-valine |
| L | L-leucine |
| I | L-isoleucine |
| M | L-methionine |
| F | L-phenylalanine |
| Y | L-tyrosine |
| W | L-tryptophan |
| H | L-histidine |
| S | L-serine |
| T | L-threonine |
| C | L-cysteine |
| N | L-asparagine |
| Q | L-glutamine |
| D | L-aspartic acid |
| E | L-glutamic acid |
| K | L-lysine |
| R | L-arginine |
| P | L-proline |
| G | glycine |
| Ava, 5-Ava | 5-aminovaleric acid |
| Als, 5-Als | 5-aminolevulinic acid |
| MeG | N-methylglycine |
| AcG | N-acetylglycine |
| Hsm | homoserine methylether |
| Har | homoarginine |
| 1nal | 1-naphthylalanine |
| 2nal | 2-naphthylalanine |
| βAla | beta-alanin |
| hoc/hcy | homocysteine |
| Ac | acetylated |
| Am | amidated |
| Dap | diamino propionic acid |
| Dab | diamino butyric acid |
| Aad | alpha-amino adipic acid |
| Asu | alpha-aminosuberic acid |
| Adi | adipic acid, |
| Glr | glutaric acid |
| Sec | selenocysteine |

As described above, the present invention also includes modifications of the peptides and defined peptide consensuses by conservative exchanges of single amino acids. Such exchanges alter the structure and function of a binding molecule but only slightly in most cases. In a conservative exchange, one amino acid is replaced by another amino acid within a group with similar properties.

Examples of Corresponding Groups are:
amino acids having non-polar side chains: A, G, V, L, I, P, F, W, M
uncharged amino acids having polar side chains: S, T, G, C, Y, N, Q
amino acids having aromatic side chains: F, Y, W
positively charged amino acids: K, R, H
negatively charged amino acids: D, E
amino acids of similar size or molecular weight, wherein the molecular weight of the replacing amino acids deviates by a maximum of +/−25% (or +/−20%, +/−15%, +/−10%) from the molecular weight of the original amino acid.

It is self evident, that the groups also include non-proteinogenic natural or non-natural amino acids with the respective side chain profile such as e.g. homoarginine in case of the group depicting positively charged side chains. In case a proline 10 substituting molecule such as e.g. a non-natural amino acid cannot be clearly assigned to one of the above groups characterized by their side-chain properties, it should usually be perceived as a non-conservative substitution of proline according to this invention. For categorizing these unusual amino acids, the classification aid according to the molecular weight might be helpful.

More specifically, Wrighton et al. (U.S. Pat. No. 5,773,569, and associated patents) examined in detail, using phage display techniques, which amino acids can be replaced, while maintaining the activity. They also investigated and published data on possible truncation, i.e. minimal length of a given EPO mimetic peptide. However, a proline near the central Gly-residue seemed to be the only possibility to obtain active peptides.

Preferably the described peptides are modified as to AcG at the N-terminus and MeG at the C-terminus.

As mentioned above, it is preferred that the peptides comprise two EPO mimetic consensus sequences and thus monomeric binding units thereby forming a dimer (or continuous bivalent peptide in case an amino acid linker is used for dimerisation). The monomeric EPO mimetic peptide units can be chosen from all of the embodiments described above in order to form the dimer. A monomeric binding unit according to the present invention may also be combined with a monomeric binding unit of EPO mimetic peptides known in the state of the art.

An EPO mimetic peptide monomer or dimer according to the present invention may further comprise at least one spacer moiety. Preferably such spacer connects the linker of a monomer or dimer to a water soluble polymer moiety or a protecting group, which may be e.g. PEG. The PEG has a preferred molecular weight of at least 3 kD, preferably between 20 and 60 kD. The spacer may be a C1-12 moiety terminated with —NH-linkages or COOH-groups and optionally substituted at one or more available carbon atoms with a lower alkyl substituent. A particularly preferred spacer is disclosed in WO 2004/100997. All documents —WO 2004/100997 and WO 2004/101606—are incorporated herein by reference. The PEG modification of peptides is disclosed in WO 2004/101600, which is also incorporated herein by reference.

There are several possible options to design a covalent linker between two peptide chains in order to arrive at di- or multimers. The peptides can be linked via amino acid side chains or via backbone extensions. Four different main dimerisation strategies to connect two EPO mimetic peptide moieties covalently are outlined subsequently as examples for suitable strategies.

1. Terminal Dimerization from C-Term to C-Term

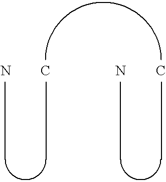

Dimerization can be achieved by means of a diketopiperazine structure at the C-terminus of each peptide. Diketoperazine linkers can be obtained by activating C-terminal amino acids, preferably glycines. The following figures show fitting examples:

### EXAMPLE a

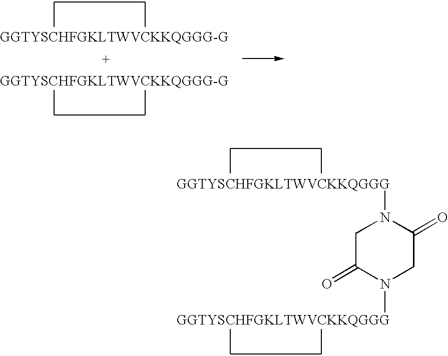

### EXAMPLE b

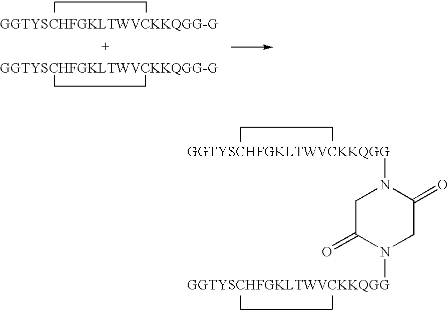

### EXAMPLE c

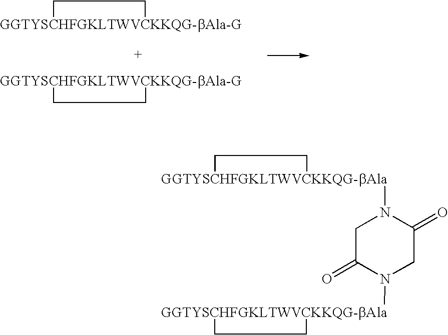

### EXAMPLE d

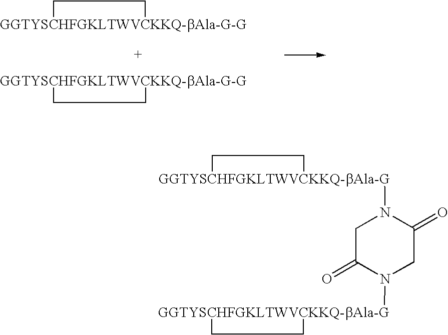

Please note that according to the second embodiment of the present invention there would be a naphthylalanine in position 13 instead of tryptophane. The above sequences are only used for describing the dimerisation principle/concept which, however, also works for other peptides described in the present application.

2. Terminal Dimerization from N-Term to N-Term

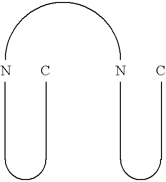

The following examples represent dimeric peptides wherein the N-terminus of one of said monomeric peptides is covalently bound to the N-terminus of the other peptide, whereby the spacer unit is preferably containing a dicarboxylic acid building block.

### EXAMPLE a

Example of a dimer containing a hexanedioyl (C6) unit as linker/spacer:

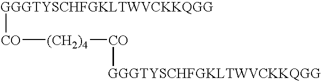

The linking bridge in this dimeric structure is custom-made by molecular modelling to avoid distortions of the bioactive conformation.

### EXAMPLE b

Example of a tailored dimer containing an octanedioyl (C8) unit as linker/spacer:

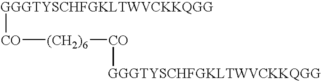

3. Dimerization Via Sidechains

Furthermore, dimerisation can occur via a covalent bond formed between the sidechains of the monomeric peptides which are supposed to form the dimer.

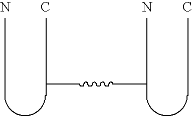

### Several Options Exist:

According to one embodiment, the side chains of the amino acid in position X₁₈ (e.g. Gln) are adjacent to each other in the EPO mimetic peptide-EPOR complex. These Gln18 side chains can be replaced by a covalent bridge. The following formulas show examples of peptide dimers linked via side chains of the amino acid in position 18:

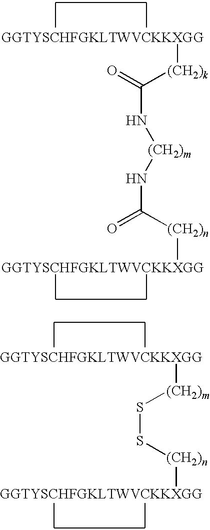

The right distance and geometry has to be considered in the design of adequate linkers.

When the geometry of the peptide with the following formula is optimized, the structure is contracted and deformated in comparison to the native peptide dimer:

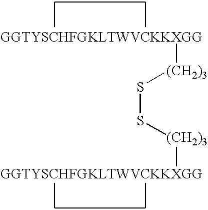

In contrast to the previous structure, a dimerization via thiolysine in position 18 does not distort the dimer substantially.

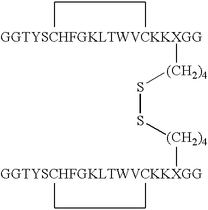

According to a different strategy, the covalent bridge linking the peptide monomers to each other thereby forming the dimer is formed between the sidechains of the C-terminal amino acid of the first monomeric peptide unit and the N-terminal amino acid of the second peptide monomer. Hence, it is preferred according to this dimerisation strategy that the EPO mimetic peptides to be dimerized carry an amino acid with a bridge forming functionality at either the N- or C-terminus thereby allowing the formation of a covalent bond between the last amino acid of the first peptide and the first amino acid of the second peptide. The bond creating the dimer is preferably covalent. Suitable examples of respective bridges are e.g. the disulfide bridge and the diselenide bridge. However, also e.g. amide bonds between positively and negatively charged amino acids or other covalent linking bonds such as thioether bonds are suitable as linking moieties (see above regarding embodiment 1).

Preferred amino acids suitable for forming respective connecting bridges were outlined in conjunction with the first embodiment of the present invention. They are e.g. cysteine, cysteine derivatives such as homocysteine or selenocysteine or thiolysine. They form either disulfide bridges or, in case of selenium containing amino acids, diselenide bridges.

Suitable examples for respectively created dimers are given below:

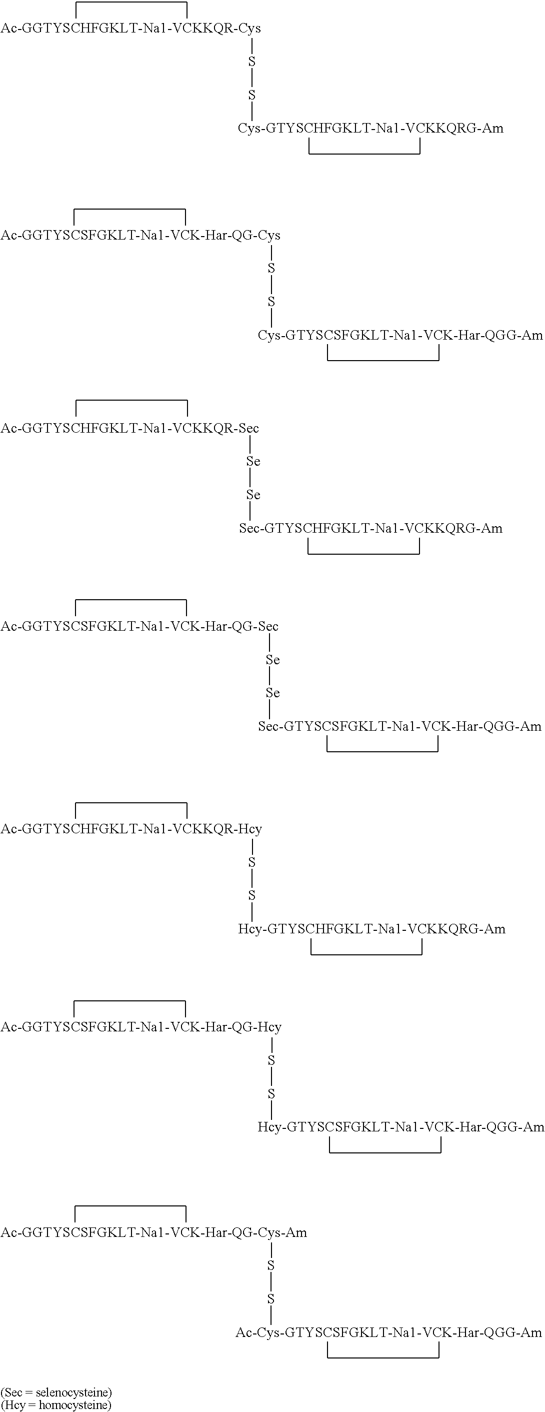

According to a further development either at the N- or the C-terminus of the peptide dimer (and hence of the respective monomeric peptide units either being located at the beginning or the end of the dimer) comprise an extra amino acid, allowing the coupling of a carrier such as HES. Consequently, the introduced amino acid carries a respective coupling functionality such as e.g. an SH-group. One common example for such an amino acid is cysteine. However, also other amino acids with a functional group allowing the formation of a covalent bond (e.g. all negatively and positively charged amino acids) are suitable.

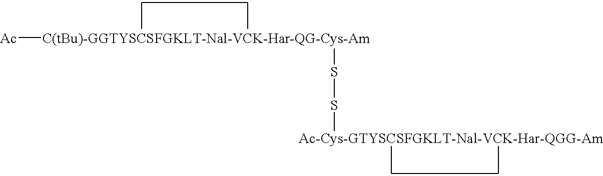

The bars over the peptide monomers represent covalent intramolecular bridges; in this case disulfide bridges.

According to a further development the amino acid at the C and/or the N terminus involved in forming the covalent bridge for connecting the monomeric units to a dimer depicts a charged group such as e.g. the COO⁻ or the NH₃⁺ group. This feature leads to a favourable stabilisation of the structure of the intermolecular bridge:

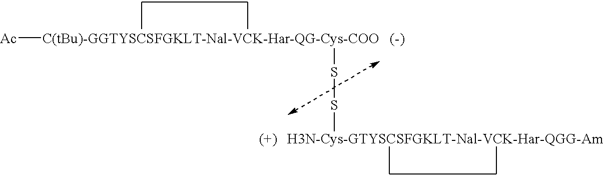

4. Continuous Bivalent Peptides

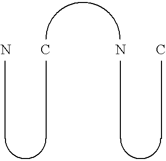

The core concept of this strategy refrains from synthesizing the monomeric peptides units in separate reactions prior to dimerization or multimerization, but to synthesize the final bi- or multivalent peptide in one step as a single continuous peptide; e.g. in one single solid phase reaction. Thus a separate dimerization or multimerization step is obsolete. This aspect provides a big advantage, i.e. the complete and independent control on each sequence position in the final peptide unit. The method allows to easily harbor at least two different receptor-specific binding domains in one continuous peptide unit due to independent control on each sequence position.

According to this embodiment the sequence of the final peptide between the binding domains (which is the “linker region”) is composed of amino acids only, thus leading to one single, continuous bi- or multivalent EPO mimetic peptide. In a preferred embodiment of the invention said peptide linker is composed of natural or unnatural amino acids which allow for a high conformational flexibility. In this regard it can be advantageous to use glycine residues as linking amino acids, which are known for their high flexibility in terms of torsion. However, also other amino acids, such as alanine or beta-alanine, or a mixture thereof can be used for creating the peptide linker. The number and choice of used amino acids depend on the respective steric facts. This embodiment of the invention allows the custom-made design of a suitable linker by molecular modeling in order to avoid distortions of the bioactive conformation. A linker composed of 3 to 5 amino acids is especially preferred.

It is noteworthy that the linker between the functional domains (or monomeric units) of the final bivalent or multivalent peptides can be either a distinct part of the peptide or can be composed—fully or in parts—of amino acids which are part of the monomeric functional domains. For example small flexible amino acids at the beginning of the peptide monomer (e.g. positions X₁ and X₂) and at the end of the peptide monomer (e.g. positions X₁₉ and X₂₀) are preferred in order to form a flexible linker and in case of a continuous bivalent peptide. Preferred amino acids in these positions are e.g. glycine or beta-alanine residues. Examples are given with Seq. 11 to 14. Thus the term “linker” is thus rather defined functionally than structurally, since an amino acid might form part of the linker unit as well as of the monomeric subunits.

Since—as mentioned above—during the synthesis of the bivalent/multivalent peptide each sequence position within the final peptide is under control and thus can be precisely determined it is possible to custom- or tailor make the peptides or specific regions or domains thereof, including the linker. This is of specific advantage since it allows the avoidance of distortion of the bioactive conformation of the final bivalent peptide due to unfavorable intramolecular interactions. The risk of distortions can be assessed prior to synthesis by molecular modeling. This especially applies to the design of the linker between the monomeric domains.

The continuous bivalent/multivalent peptides having a peptide linker for dimerisation show much higher activity then the corresponding monomeric peptides and thus confirm the observation known from other dimeric peptides that an increase of efficacy is associated with bivalent peptide concepts.

The continuous bivalent/multivalent peptides can be modified by e.g. acetylation or amidation or be elongated at C-terminal or N-terminal positions. The prior art modifications for the monomeric peptides (monomers) mentioned above including the attachments of soluble moieties such as PEG, starch or dextrans are also applicable for the multi- or bivalent peptides according to the invention.

All possible modifications also apply for modifying the linker. In particular it might be advantageous to attach soluble polymer moieties to the linker such as e.g. PEG, starch or dextrans.

The synthesis of the final multi- or bivalent peptide according to the invention favorably can also include two subsequent and independent formations of disulfide bonds or other intramolecular bonds within each of the binding domains. Thereby the peptides can also be cyclized.

The bivalent structures according to the invention are favorably formed on the basis of the peptide monomers reported herein.

The reactive side chains of the peptides may serve as a linking tie e.g. for further modifications. The dimeric peptides furthermore optionally comprise intramolecular bridges between the first and second and/or third and fourth amino acid having a bridge forming side chain functionality (X₆ and X₁₅) such as e.g. the cysteines.

The peptides can be modified by e.g. acetylation or amidation or can be elongated at the C-terminal or N-terminal positions. Extension with one or more amino acids at one of the two termini (N or C), e.g. for preparation of an attachment site for a polymer often leads to a heterodimeric bivalent peptide unit which can best be manufactured as a continuous peptide.

Several reactive amino acids are known in the state of the art in order to couple carriers to protein and peptides. A preferred coupling amino acid is cysteine which can be either coupled to the N or C terminus. However, the coupling direction can make a considerable difference and should thus be carefully chosen for each peptide. This shall be demonstrated on the basis of the following example:

Used are the following two dimers:

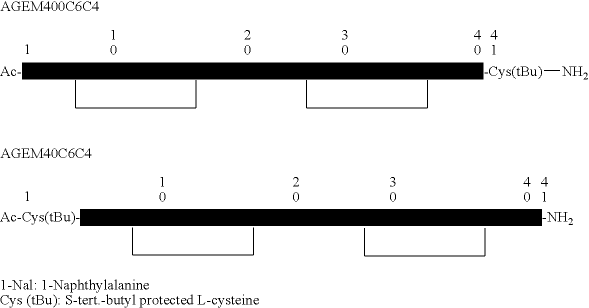

The 41mers AGEM400C6C4 and AGEM40C6C4 posses the same core sequence. The amino acids 1-40 of AGEM40C6C4 equal the amino acids 2-41 of AGEM40C6C4. The only difference is the position of the tBu-protected cysteine. This amino acid is not involved in the receptor drug interaction but is destined to function as the linking group to a polymeric carrier in the final conjugate. In case of AGEM400C6C4 the tBu-protected cysteine is attached to the C term, in case of AGEM40C6C4 it is attached to the N term. The connecting bars represent cysteine bridges.

There are two advantages of AGEM400C6C4 over AGEM40C6C4.

The first advantage is its synthetic accessibility. AGEM400C6C4 can be isolated in higher overall yields than AGEM40C6C4. In case of the synthesis of the linear sequence of AGEM40C6C4 a CIZ-22mer (CIZ-RGGGTYSCHFGKLT-1-NaI-VCKKQRG-NH₂, CIZ: 2-Chlorobenzyloxycarbonyl group) is observed as a byproduct. During purification of the linear sequence with reversed phase high pressure liquid chromatography (RP-HPLC) it exhibits a similar chromatographic behaviour as the linear precursor of AGEM40C6C4 and therefore makes it difficult to be separated from it leading to a loss in overall yield of the desired product. In case of AGEM400C6C4 no analogous compound is found.

The second advantage of AGEM400C6C4 over AGEM40C6C4 lies in the easier implementation of an analysis of the final conjugate of the deprotected peptide with a polymeric carrier. One strategy for the analysis of a peptide conjugate is the selective degradation of the conjugate by cleavage with endoproteases. Ideally the whole peptide is released from the polymeric carrier during the enzymatic hydrolysis. These peptide fragments can be identified and quantified by standard analytical techniques like i.e. HPLC with UV or MS detection, etc.

In case of AGEM400C6C4 the cleavage can be affected with trypsine—an endoprotease that is known to cleave highly selectively peptide bonds that lie C terminal of the charged amino acids arginine and lysine (F. Lottspeich, H. Zorbas (Hrsg.), “Bioanalytik”, Spectrum Akademischer Verlag, Heidelberg, Berlin, 1998). Applied to conjugates of AGEM400C6C4 this will set free fragments that cover 38 of 41 amino acids of the original peptide bound to the carrier molecule. In case of AGEM40C6C4 fragments of only 21 of 41 amino acids are released by the tryptic digest:

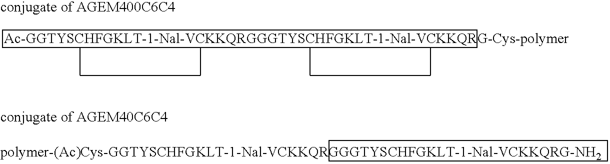

Fragments that are set free and can be detected by follow-up analyses are marked grey.

As the analysis of an Active Pharmaceutical Ingredient is a key issue during its development AGEM400C6C4 has a clear advantage over AGEM40C6C4.

Thus in case a positively charged amino acid is located in the respective positions, it is highly preferred to incorporate the linking amino acid (here cysteine) at the C-terminus because it possible to generate a nearly complete peptide fragment since a cleavage site is due to the arginine in position X₁₉ of the monomer pretty much right before the polymer.

The compounds of the present invention can advantageously be used for the preparation of human and/or veterinarian pharmaceutical compositions. They are thus suitable for use in human and veterinarian therapy. As EPO mimetics they depict the basically the same qualitative activity pattern as erythropoietin. They are thus generally suitable for the same indications as erythropoietin.

Erythropoietin is a member of the cytokine super family. Besides the stimulating effects described in the introduction, it was also found that erythropoietin stimulates stem cells. The EPO mimetics described herein are thus suitable for all indications caused by stem cell associated effects. Non-limiting examples are the prevention and/or treatment of diseases associated with the nerve system. Examples are neurological injuries, diseases or disorders, such as e.g. Parkinsonism, Alzheimer's disease, Huntington's chorea, multiple sclerosis, amyotrophic lateral sclerosis, Gaucher's disease, Tay-Sachs disease, a neuropathy, peripheral nerve injury, a brain tumor, a brain injury, a spinal cord injury or a stroke injury. The EPO mimetic peptides according to the invention are also usable for the preventive and/or curative treatment of patients suffering from, or at risk of suffering from cardiac failure. Examples are cardiac infarction, coronary artery disease, myocarditis, chemotherapy treatment, alcoholism, cardiomyopathy, hypertension, valvar heart diseases including mitral insufficiency or aortic stenosis, and disorders of the thyroid gland, chronic and/or acute coronary syndrome.

Furthermore, the EPO mimetics can be used for stimulation of the physiological mobilization, proliferation and differentiation of endothelial precursor cells, for stimulation of vasculogenesis, for the treatment of diseases related to a dysfunction of endothelial precursor cells and for the production of pharmaceutical compositions for the treatment of such diseases and pharmaceutical compositions comprising said peptides and other agents suitable for stimulation of endothelial precursor cells. Examples of such diseases are hypercholesterolaemia, diabetis mellitus, endothel-mediated chronic inflammation diseases, endotheliosis including reticulo-endotheliosis, atherosclerosis, coronary heart disease, myocardic ischemia, angina pectoris, age-related cardiovascular diseases, Raynaud disease, pregnancy induced hypertonia, chronic or acute renal failure, heart failure, wound healing and secondary diseases.

Furthermore, the peptides according to the invention are suitable carriers for delivering agents across the blood-brain barrier and can be used for respective purposes and/or the production of respective therapeutic conjugation agents capable of passing the blood-brain barrier.

The peptides described herein are especially suitable for the treatment of disorders that are characterized by a deficiency of erythropoietin or a low or defective red blood cell population and especially for the treatment of any type of anemia or stroke. The peptides are also suitable for increasing and/or maintaining hematocrit in a mammal. Such pharmaceutical compositions may optionally comprise pharmaceutical acceptable carriers in order to adopt the composition for the intended administration procedure. Suitable delivery methods as well as carriers and additives are for example described in WO 2004/101611 and WO 2004/100997.

As outlined above, dimerization of the monomeric peptides to dimers or even multimers usually improves the EPO mimetic agonist activity compared to the respective monomeric peptides. However, it is desirable to further enhance activity. For example, even dimeric EPO mimetic peptides are less potent than the EPO regarding the activation of the cellular mechanisms.

Several approaches were made in the prior art in order to increase the activity of the peptides, for example by variation of the amino acid sequence in order to identify more potent candidates. However, so far it is still desirable to further enhance the activity of peptides, especially of EPO mimetic peptides in order to improve the biological activity.

A further embodiment of the present invention provides a solution to that problem. Therein a compound is provided that binds target molecules and comprises

i) at least two peptide units wherein each peptide unit comprises at least two domains with a binding capacity to the target;
ii) at least one polymeric carrier unit;
wherein said peptide units are bound to said polymeric carrier unit.

Surprisingly, it has been found that the combination of two or more bi- or multivalent peptides according to the invention on a polymeric support is greatly increasing the efficacy of the bivalent (or even multivalent) peptides to their binding receptor not only additively, but even over-additively. Thus a synergistic effect is observed.

The term “bivalent” as used for the purpose of the present invention is defined as a peptide comprising two domains with a binding capacity to a target, here in particular the EPO receptor. It is used interchangeably with the term “dimeric”. Accordingly, a “multivalent” or “multimeric” EPO mimetic peptide has several respective binding domains for the EPO receptor. It is self-evident that the terms “peptide” and “peptide unit” do not incorporate any restrictions regarding size and incorporate oligo- and polypeptides as well as proteins.

Compounds comprising two or more bi- or multivalent peptide units attached to a polymeric carrier unit are named “supravalent” in the context of this embodiment. These supravalent molecules greatly differ from the dimeric or multimeric molecules known in the state of the art. The state of the art combines merely monomeric EPO mimetic peptides in order to create a dimer. In contrast the supravalent molecules are generated by connecting already (at least) bivalent peptide units to a polymeric carrier unit thereby creating a supravalent molecule (examples are given in figs.). Thereby the overall activity and efficacy of the peptides is greatly enhanced thus decreasing the EC50 dose.

So far the reasons for the great potency of the supravalent molecules compared to the molecules known in the state of the art are not fully understood. It might be due to the fact that the dimeric molecules known in the state of the art provide merely one target respectively receptor binding unit per dimer. Thus only one receptor complex is generated upon binding of the dimeric compound thereby inducing only one signal transduction process. E.g. two monomeric EPO mimetic peptides are connected via PEG to form a peptide dimer thereby facilitating dimerisation of the receptor monomers necessary for signal transduction (Johnson et. al., 1997). In contrast, the supravalent compounds according to the invention comprise several already di- or multimeric respective receptor binding units. This might allow the generation of several receptor complexes on the cell surface per compound molecule thereby inducing several signal transductions and thereby potencing the activity of the peptide units over-additively. Binding of the supravalent compounds might result in a clustering of receptor complexes on the cell-surface.

The EPO mimetic peptide units used in this embodiment can be either homo- or heterogenic, meaning that either identical or differing peptide units are used. The same applies to the binding domains (monomeric peptides as described above) of the peptide units which can also be homo- or heterogenic. The bi- or multivalent peptide units bound to the carrier unit bind the same receptor target. However, they can of course still differ in their amino acid sequence. The monomeric binding domains of the bi- or multivalent peptide units can be either linear or cyclic. A cyclic molecule can be for example created by the formation of intramolecular cysteine bridges (see above).

The polymeric carrier unit comprises at least one natural or synthetic branched, linear or dendritic polymer. The polymeric carrier unit is preferably soluble in water and body fluids and is preferably a pharmaceutically acceptable polymer. Water soluble polymer moieties include, but are not limited to, e.g. polyalkylene glycol and derivatives thereof, including PEG, PEG homopolymers, mPEG, polypropyleneglycol homopolymers, copolymers of ethylene glycol with propylene glycol, wherein said homopolymers and copolymers are unsubstituted or substituted at one end e.g. with an acylgroup; polyglycerines or polysialic acid; cellulose and cellulose derivatives, including methylcellulose and carboxymethylcellulose; starches (e.g. hydroxyalkyl starch (HAS), especially hydroxyethyl starch (HES) and dextrines, and derivatives thereof; dextran and dextran derivatives, including dextransulfat, crosslinked dextrin, and carboxymethyl dextrin; chitosan (a linear polysaccharide) heparin and fragments of heparin; polyvinyl alcohol and polyvinyl ethyl ethers; polyvinylpyrrollidon; alpha,beta-poly[(2-hydroxyethyl)-DL-aspartamide; and polyoxyethylated polyols. One example of a carrier unit is a homobifunctional polymer, of for example polyethylene glycol (bis-maleimide, bis-carboxy, bis-amino etc.).

The polymeric carrier unit which is coupled to at least two dimeric EPO mimetic peptides comprising monomeric consensus sequences according to the present invention can have a wide range of molecular weight due to the different nature of the different polymers that are suitable in conjunction with the present invention. There are thus no size restrictions. However, it is preferred that the molecular weight is at least 3 kD, preferably at least 10 kD and approximately around 20 to 500 kD and more preferably around 30 to 150 or around 60 or 80 kD. The size of the carrier unit depends on the chosen polymer and can thus vary. For example, especially when starches such as hydroxyethylstarch are used, the molecular weight might be considerably higher. The average molecular weight might then be arranged around 100 to 4,000 kD or even be higher. However, it is preferred that the molecular weight of the HES molecule lies around 50 to 500 kD, or 100 to 300 kD and preferably around 200 kD. The size of the carrier unit is preferably chosen such that each peptide unit is optimally arranged for binding their respective receptor molecules.

In order to facilitate this, one embodiment of the present invention uses a carrier unit comprising a branching unit. According to this embodiment, the polymers, as for example PEG, are attached to a branching unit thus resulting in a large carrier molecule allowing the incorporation of numerous peptide units. Examples for appropriate branching units are glycerol or polyglycerol. Also dendritic branching units can be used as for example taught by Haag 2000, herein incorporated by reference. Also the HES carrier may be used in a branched form. This e.g. if it is obtained to a high proportion from amylopectin.

Preferably, after the peptide units are created by combining the monomeric binding units to peptide units (either head to head, head to tail, or tail to tail) the polymeric carrier unit is connected to the peptide units. The polymeric carrier unit is connected/coupled to the peptide units via a covalent or a non-covalent (e.g. a coordinative) bond. However the use of a covalent bond is preferred. The attachment can occur e.g. via a reactive amino acid of the peptide units e.g. lysine, cysteine, histidine, arginine, aspartic acid, glutamic acid, serine, threonine, tyrosine or the N-terminal amino group and the C-terminal carboxylic acid. In case the peptide does not carry a respective amino acid, such an amino acid can be introduced into the amino acid sequence. The coupling should be chosen such that the binding to the target is not or at least as little as possible hindered. Depending on the conformation of the peptide unit, the reactive amino acid is either at the beginning, the end or within the peptide sequence.

In case the polymeric carrier unit does not possess an appropriate coupling group, several coupling substances/linkers can be used in order to appropriately modify the polymer in order that it can react with at least one reactive group on the peptide unit to form the supravalent compound. Suitable chemical groups that can be used to modify the polymer are e.g. as follows:

Acylating groups which react with the amino groups of the protein, for example acid anhydride groups, N-acylimidazole groups, azide groups, N-carboxy anhydride groups, diketene groups, dialkyl pyrocarbonate groups, imidoester groups, and carbodiimide-activated carboxyl-groups. All of the above groups are known to react with amino groups on proteins/peptides to form covalent bonds, involving acyl or similar linkages;

alkylating groups which react with sulfhydryl (mercapto), thiomethyl, imidazo or amino groups on the peptide unit, such as halo-carboxyl groups, maleimide groups, activated vinyl groups, ethylenimine groups, aryl halide groups, 2-hydroxy 5-nitro-benzyl bromide groups; and aliphatic aldehyde and ketone groups together with reducing agents, reacting with the amino group of the peptide;
ester and amide forming groups which react with a carboxyl group of the peptide, such as diazocarboxylate groups, and carbodiimide and amine groups together;
disulfide forming groups which react with the sulfhydryl groups on the protein, such as 5,5′-dithiobis(2-nitrobenzoate) groups, ortho-pyridyl disulfides and alkylmercaptan groups (which react with the sulfhydryl groups of the protein in the presence of oxidizing agents such as iodine);
dicarbonyl groups, such as cyclohexandione groups, and other 1,2-diketone groups which react with the guanidine moieties of the peptide;
diazo groups, which react with phenolic groups on the peptide;
reactive groups from reaction of cyanogens bromide with the polysaccharide, which react with amino groups on the peptide.

Thus in summary, the compound according to the invention may be made by—optionally—first modifying the polymeric carrier chemically to produce a polymeric carrier having at least one chemical group thereon which is capable of reacting with an available or introduced chemical group on the peptide unit, and then reacting together the—optionally—modified polymer and the peptide unit to form a covalently bonded complex thereof utilising the chemical group of the—if necessary—modified polymer.

In case coupling occurs via a free SH-group of the peptide (e.g. of a cysteine group), the use of a maleimide group in the polymer is preferred.

In order to generate a defined molecule it is preferred to use a targeted approach for attaching the peptide units to the polymeric carrier unit. In case no appropriate amino acids are present at the desired attachment site, appropriate amino acids can be incorporated in the dimeric EPO mimetic peptide unit. For site specific polymer attachment a unique reactive group e.g. a specific amino acid at the end of the peptide unit is preferred in order to avoid uncontrolled coupling reactions throughout the peptide leading to a heterogeneous mixture comprising a population of several different polymeric molecules.

The coupling of the peptide units to the polymeric carrier unit, e.g. PEG or HES, is performed using reactions principally known to the person skilled in the art. E.g. there are number of PEG and HES attachment methods available to those skilled in the art (see for example WO 2004/100997 giving further references, Roberts et al., 2002; U.S. Pat. No. 4,064,118; EP 1 398 322; EP 1 398 327; EP 1 398 328; WO 2004/024761; all herein incorporated by reference).

It is important to understand that the concept of supravalency described herein is different from the known concept of PEGylation or HESylation. In the state of the art e.g. PEGylation is only used in order to produce either peptide dimers or in order to improve pharmacokinetic parameters by attaching one or more PEG units to a peptide. However, as outlined above, the attachment of two or more at least bivalent peptide units to e.g. PEG or HES as a polymeric carrier unit also greatly enhances efficacy (thus decreasing the EC50-dose). The concept of this invention thus has strong effects on pharmacodynamic parameters and not only on pharmacokinetic parameters as it is the case with the PEGylation or HESylation concepts known in the state of the art. However, of course the incorporation of for example PEG or HES as polymeric carrier unit also has the known advantages regarding pharmacokinetics:

PEGylation is usually undertaken to improve the biopharmaceutical properties of the peptides. The most relevant alterations of the protein molecule following PEG conjugation are size enlargement, protein surface and glycosylation function masking, charge modification and epitope shielding. In particular, size enlargement slows down kidney ultrafiltration and promotes the accumulation into permeable tissues by the passive enhance permeation and retention mechanism. Protein shielding reduces proteolysis and immune system recognition, which are important routes of elimination. The specific effect of PEGylation on protein physicochemical and biological properties is strictly determined by protein and polymer properties as well as by the adopted PEGylation strategy.

However, the use of PEG or other non-biodegradable polymers might lead to new problems.

During in vivo applications, dosage intervals in a clinical setting are triggered by loss of effect of the drug. Routine dosages and dosage intervals are adapted such that the effect is not lost during dosage intervals. Due to the fact that peptides attached to a non-biodegradable, large polymer unit (e.g. a PEG-moiety) can be degraded faster than the support molecule might be eliminated by the body, a risk of accumulation of the carrier unit can arise. Such a risk of accumulation always occurs as effect-half life time of the drug is shorter than elimination half life time of the drug itself or one of its components/metabolites. Thus, accumulation of the carrier molecule should be avoided especially in long-term treatments because peptides are usually PEGylated with very large PEG-moieties (˜20-40 kD) which thus show a slow renal elimination. The peptide moiety itself undergoes enzymatic degradation and even partial cleavage might suffice to deactivate the peptide.

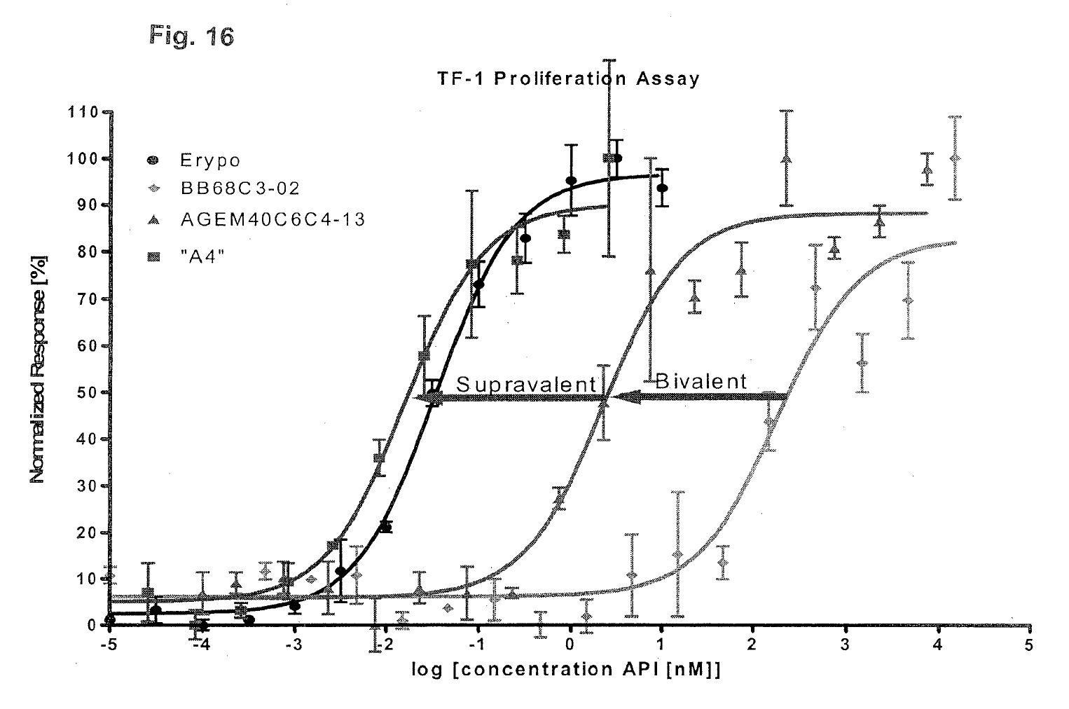

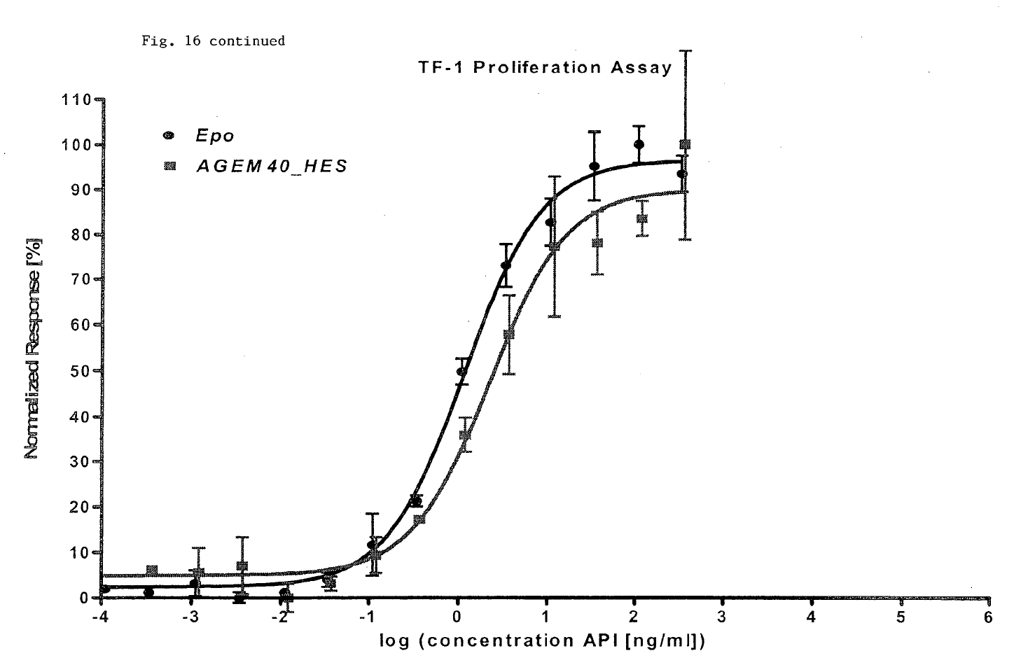

In order to find a solution to this potential problem one embodiment of the present invention teaches the use of a polymeric carrier unit that is composed of at least two subunits. The polymeric subunits are connected via biodegradable covalent linker structures. According to this embodiment the molecular weight of the large carrier molecule (for example 40 kD) is created by several small or intermediate sized subunits (for example each subunit having a molecular weight of 5 to 10 kD), that are connected via biodegradable linkers. The molecular weights of the modular subunits add up thereby generating the desired molecular weight of the carrier molecule. However, the biodegradable linker structures can be broken up in the body thereby releasing the smaller carrier subunits (e.g. 5 to 10 kD). The small carrier subunits show a better renal clearance than a polymer molecule having the overall molecular weight (e.g. 40 kD). An illustrating example is given in FIG. 16.

The linker structures are selected according to known degradation properties and time scales of degradation in body fluids. The breakable structures can, for instance, contain cleavable groups like carboxylic acid derivatives as amide/peptide bonds or esters which can be cleaved by hydrolysis (see e.g. Roberts, 2002 herein incorporated by reference). PEG succinimidyl esters can also be synthesized with various ester linkages in the PEG backbone to control the degradation rate at physiological pH (Zhao, 1997, herein incorporated by reference). Other breakable structures like disulfides of benzyl urethanes can be cleaved under mild reducing environments, such as in endosomal compartments of a cell (Zalipsky, 1999) and are thus also suitable. Other criteria for selection of appropriate linkers are the selection for fast (frequently enzymatic) degradation or slow (frequently non-enzymatic decomposition) degradation. Combination of these two mechanisms in body fluids is also feasible. It is clear that this highly advantageous concept is not limited to the specific peptide units described or referred to herein but also applies to other pharmaceutical molecules that are attached to large polymer units such as PEG molecules wherein the same problems of accumulation arises.

According to one embodiment hydroxyalkylstarch and preferably HES is used as polymeric carrier unit. HES has several important advantages. First of all, HES is biodegradable. Furthermore, the biodegradability of HES can be controlled via the ratio of hydroxyethyl groups and can thus be influenced. A molar degree of substitution of 0.4-0.8 (in average 40-80% of the glucose units contain a hydroxyethyl group) are well suitable for the purpose of the present invention. Due to the biodegradability, accumulation problems as described above in conjunction with PEG do usually not occur. Furthermore, HES has been used for a long time in medical treatment e.g. in form of a plasma expander. Its innocuousness is thus approved.

Furthermore, derivatives of hydrolysis products of HES are detectable by gas chromatography. HES-peptide conjugates can be hydrolysed under conditions under which the peptide units are still stable. This allows the quantification and monitoring of the degradation products and allows evaluations and standardisations of the active peptides.

According to a further embodiment a first type of polymeric carrier unit is used and loaded with peptide units. This first carrier is preferably easily biodegradable as is e.g. HES. However, not all attachment spots of the first carrier are occupied with peptide units but only e.g. around 20 to 50%. Depending on the size of the used polymer, several hundred peptide units could generally be coupled to the carrier molecule. However, usually less peptide units are used, such as 2 to 50 or 2 to 20. 2 to 15, 2 to 10, 2 to 8 and 3 to 6 peptides are preferred for EPO mimetic peptides. The rest (or at least some) of the remaining attachment spots of the first carrier are occupied with a different carrier, e.g. small PEG units having a lower molecular weight than the first carrier. This embodiment has the advantage that a supravalent composition is created due to the first carrier which is however, very durable due to the presence of the second carrier, which is constituted preferably by PEG units of 3 to 5 or 10 kD. However, the whole entity is very well degradable, since the first carrier (e.g. HES) and the peptide units are biodegradable and the second carrier, e.g. PEG is small enough to be easily cleared from the body.

The monomers constituting the binding domains of the peptide units recognize the homodimeric erythropoietin receptor. The latter property of being a homodimeric receptor differentiates the EPO-receptor from many other cytokine receptors. The peptide units comprising at least two EPO mimetic monomeric binding domains as described above bind the EPO receptor and preferably are able to di-respectively multimerise their target and/or stabilize it accordingly thereby creating a signal transduction inducing complex.

The present invention also comprises respective compound production methods, wherein the peptide units are connected to the respective carrier units. The present invention furthermore comprises respective compound production methods, wherein the peptide units are connected to the respective polymeric carrier units. The compounds of the present invention can advantageously be used for the preparation of human and/or veterinarian pharmaceutical compositions. They can be especially suitable for the treatment of disorders that are characterized by a deficiency of erythropoietin or a low or defective red blood cell population and especially for the treatment of any type of anemia and stroke. They are also usable for all indications described above. Such pharmaceutical compositions may optionally comprise pharmaceutical acceptable carriers in order to adopt the composition for the intended administration procedure. Suitable delivery methods as well as carriers and additives are for example described in WO 2004/100997 and WO 2004/101611, herein incorporated by reference.

### EXAMPLES

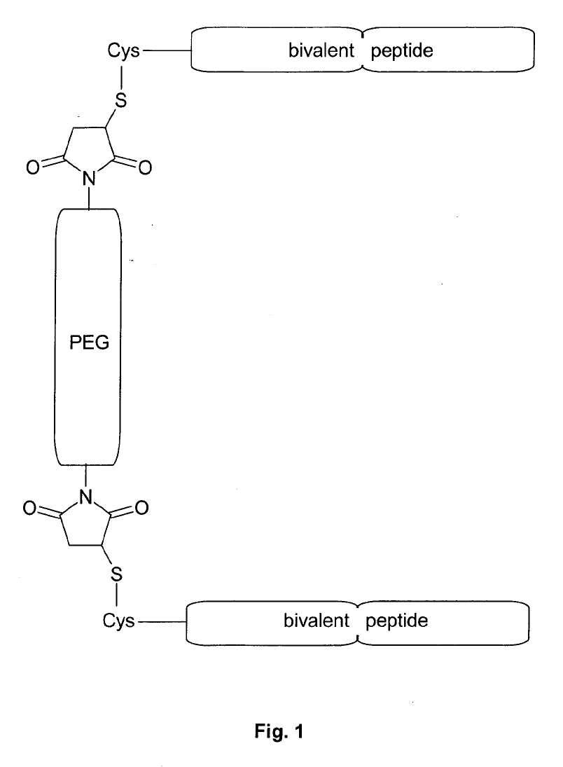

The concept of the supravalent molecules shall be explained by means of examples. FIG. 1 shows an example of a simple supravalent molecule according to the invention. Two continuous bivalent peptides are connected N-terminally by a bifunctional PEG moiety carrying maleimide groups. Cysteine was chosen as reactive attachment site for the PEG carrier unit.

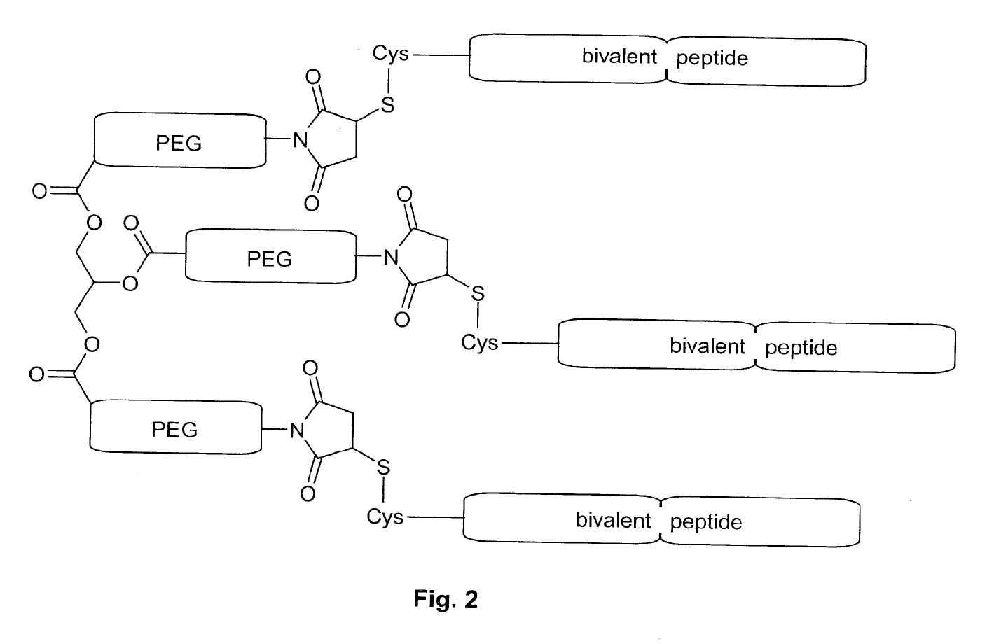

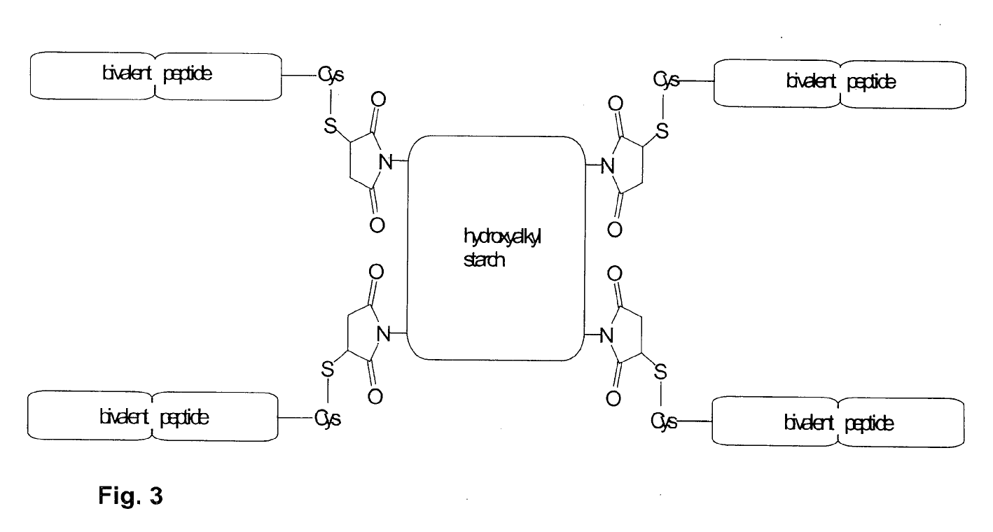

However, supravalent molecules can comprise more than two continuous bi- or multivalent peptide units. FIG. 2 gives an example that is based on a carrier unit with a central glycerol unit as branching unit and comprising three continuous bivalent peptides. Again cysteine was used for attachment. FIG. 3 shows an example using HES as polymeric carrier unit. HES was modified such that it carries maleimide groups reacting with the SH groups of the peptide units. According to the example, all attachment sites are bound to peptide units (here 4). However, also small PEG units (e.g. 3 to 10 kD) could occupy at least some of the attachment sites.

As explained above, the supravalent concept can also be extended to polyvalent dendritic polymers wherein a dendritic and/or polymer carrier unit is connected to a larger number of continuous bivalent peptides. For example, the dendritic branching unit can be based on polyglycerol (please refer to Haag 2000, herein incorporated by reference).

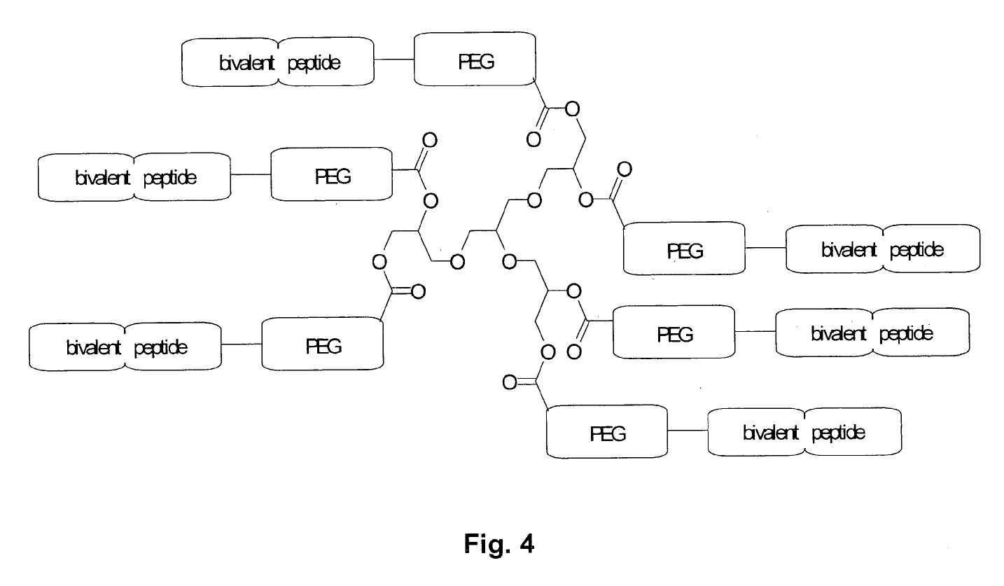

An example for a supravalent molecule based on a carrier unit with a dendritic branching unit containing six continuous bivalent peptides is shown in FIG. 4.

Other examples of supravalent molecules comprise carrier units with starches or dextrans, which are oxidized using e.g. periodic acid to harbor a large number of aldehyde functions. In a second step, many bivalent peptides are attached to the carrier unit and together form the final molecule. Please note that even several hundred (e.g. 50 to 1000, preferably 150 to 800, more preferably 250 to 700) peptide units can be coupled to the carrier molecule, which is e.g. HES. However, also far less peptide units may be bound to the HES molecule as it is shown in the Figs., especially if EPO mimetic peptides are coupled. The average number of peptide units to be coupled may be chosen from around 2 to 1000, 2 to 500, 2 to 100, 2 to 50, preferably 2 to 20 and most preferably 2 to 10, depending on the peptide and the receptor(s) to be bound.

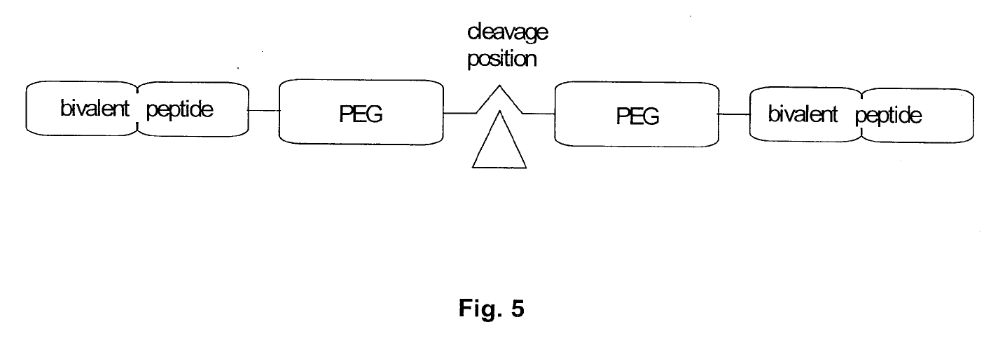

FIG. 5 demonstrates the concept of a simple biodegradable supravalent molecule. Two continuous bivalent peptides are connected N-terminally by two bifunctional PEG moieties that are connected via a biodegradable linker having an intermediate cleavage position. The linkers allow the break up of the large PEG unit in the subunits thereby facilitating renal clearance.

The advantages connected to the supravalence effect were very surprising and unexpected. Initially it was feared, that the conjugation to a macromolecule might reduce efficacy. This expectation was based on the assumed disadvantages in binding rate due to reduced diffusion rates with larger molecules. Another expectation was, that from the several peptide APIs bound to a carrier not all would be able to bind to the receptor potentially due to sterical problems of simultaneous binding or because the number of receptors, which can be reached by the extensions of the macromolecular carrier is limited and possibly below the number of peptide APIs. Thus, an increase of potency of the peptide API (Active Pharmaceutical Ingredient) as is seen with the supravalence concept of the present invention was not expected.

On the other side, due to the significant pharmacokinetic changes a macromolecular carrier is able to introduce, the in vivo potency could have been improved due to the longer half life time of the whole Peptide/Carrier complex. This phenomenon also has the effect that a supravalence effect is difficult to determine in vivo, since it is a pharmacodynamic entity, which has to be determined separately. In vitro assays are thus not only sufficient, but might be the only useful way of clearly demonstrating the supravalence effect.

The supravalence effect as described in this invention can be demonstrated by comparison of molar amounts of peptide API (conjugated to a carrier vs. unconjugated).

An experiment was performed in a standard TF-1 cell assay as recommended by the European Pharmacopoe for the determination of EPO-like activity in vitro (please also see below). Basically, TF-1 cells (their proliferation being dependent from the presence of EPO-like activity) are cultured in the presence various concentrations of EPO or EPO-mimetic substances. The resulting cell numbers are quantified using colorimetric MTT-assay and photometric measurements.

Based on these data, it is possible to determine normalized dose-response relations for each given substance.

In this assay EPO and the peptide AGEM40 (see below), the latter being a continuous bivalent peptide with EPO-mimetic activity was used.

AGEM40 was used as unconjugated peptide and as peptide conjugated to macromolecular carrier (in this case hydroxyethylstarch of the mean molecular weight 130 kD). The Building Block Size of this conjugate is roughly 40 kD, which means that the average HES-molecule carries about 2-5, preferably 2 to 4 peptide moieties. Also a HES 200/0.5 may be used. After modification of the 130 kD HES approximately 4 peptides were conjugated. When a HES having a molecular weight of 200 kD was used, this amounts to approx. 5 peptide units conjugated to the HES.

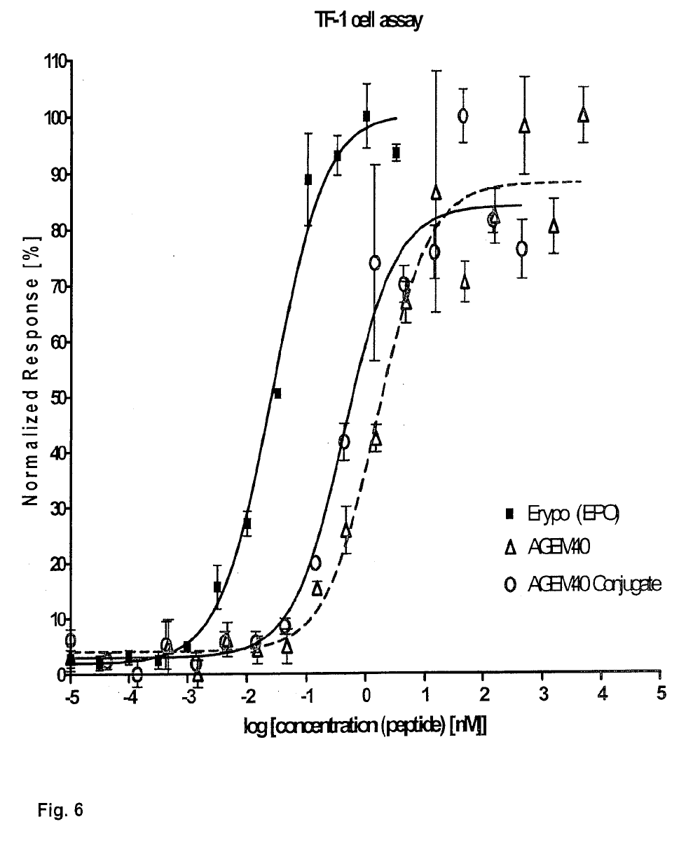

The comparison shown in FIG. 6 is based on molar comparison of peptide concentration, whether or not the peptide is conjugated. In contrast to the expectations, potency is increasing (EC50 is decreasing and the dose response curve is situated left from the unconjugated peptide) thereby demonstrating a positive pharmacodynamic influence of oligovalent conjugation to a macromolecular carrier.

Thus—independent from the expected pharmacokinetic improvements—the conjugation concept according to the invention clearly increases potency of the overall active pharmaceutical ingredient.

This is a new mechanism, which can certainly be used for peptides addressing the EPO-receptor, but potentially also for other membrane bound pharmacological targets, especially other cytokine receptors such as those for thrombopoietin, G-CSF, interleukins, and others.

### I. Peptide Synthesis of Monomers

### Manual Synthesis

The synthesis is carried out by the use of a Discover microwave system (CEM) using PL-Rink-Amide-Resin (substitution rate 0.4 mmol/g) or preloaded Wang-Resins in a scale of 0.4 mmol. Removal of Fmoc-group is achieved by addition of 30 ml piperidine/DMF (1:3) and irradiation with 100 W for 3×30 sec. Coupling of amino acids is achieved by addition of 5 fold excess of amino acid in DMF PyBOP/HOBT/DIPEA as coupling additives and irradiation with 50 W for 5×30 sec. Between all irradiation cycles the solution is cooled manually with the help of an ice bath. After deprotection and coupling, the resin is washed 6 times with 30 ml DMF. After deprotection of the last amino acid some peptides are acetylated by incubation with 1.268 ml of capping solution (4.73 ml acetic anhydride and 8.73 ml DIEA in 100 ml DMSO) for 5 minutes. Before cleavage, the resin is then washed 6 times with 30 ml DMF and 6 times with 30 ml DCM. Cleavage of the crude peptides is achieved by treatment with 5 ml TFA/TIS/EDT/H₂O (94/1/2.5/2.5) for 120 minutes under inert atmosphere. This solution is filtered into 40 ml cold ether. The precipitate is dissolved in acetonitrile/water (1/1) and the peptide is purified by RP-HPLC (Kromasil 100 C18 10 μm, 250×4.6 mm).

### Automated Synthesis

The synthesis is carried out by the use of an Odyssey microwave system (CEM) using PL-Rink-Amide-Resins (substitution rate 0.4 mmol/g) or preloaded Wang-Resins in a scale of 0.25 mmol. Removal of Fmoc-groups is achieved by addition of 10 ml piperidine/DMF (1:3) and irradiation with 100 W for 10×10 sec. Coupling.) of amino acids is achieved by addition of 5 fold excess of amino acid in DMF PyBOP/HOBT/DIPEA as coupling additives and irradiation with 50 W for 5×30 sec. Between all irradiation cycles the solution is cooled by bubbling nitrogen through the reaction mixture. After deprotection and coupling, the resin is washed 6 times with 10 ml DMF. After deprotection of the last amino acid, some peptides are acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 ml TFA/TIS/EDT/H₂O (941112.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

### Purification

All peptides were purified using a Nebula-LCMS-system (Gilson). The crude material of all peptides was dissolved in acetonitrile/water (1/1) and the peptide purified by RP-HPLC (Kromasil 100 C18 10 μm, 250×4.6 mm). The flow rate was 20 ml/min and the LCMS split ratio 1/1000.

### II. Formation of Intramolecular Disulfide Bridges

Cyclization with K₃-[(FeCN₆)

Solution1: 10 mg of the peptide are dissolved in 0.1% TFA/acetonitrile and diluted with water until a concentration of 0.5 mg/ml is reached. Solid ammonium bicarbonate is added to reach a pH of app. 8.

Solution 2: In a second vial 10 ml 0.1% TFA/acetonitrile are diluted with 10 ml of water. Solid ammonium bicarbonate is added until a pH of 8 is reached and 1 drop of a 0.1M solution of K₃[(FeCN₆)] is added.

Solution 1 and 2 are added dropwise over a period of 3 hours to a mixture of acetonitrile/water (1/1; pH=8). The mixture is incubated at room temperature overnight and the mixture concentrated and purified by LCMS.

Cyclization with CLEAR-OX™-Resin

To 100 ml of acetonitrile/water (1/1; 0.1% TFA), solid ammonium bicarbonate is added until a pH of 8 is reached. This solution is degassed by bubbling Argon for 30 minutes. Now 100 mg of CLEAR-OX™-resin is added. After 10 minutes, 10 mg of the peptide is added as a solid. After 2 h of incubation, the solution is filtered, concentrated and purified by LCMS.

### Purification of Cyclic Peptides:

All peptides were purified using a Nebula-LCMS-system (Gilson). The crude material of all peptides was dissolved in acetonitrile/water (1/1) or DMSO and the peptide was purified by RP-HPLC (Kromasil 100 C18 or C8 10 μm, 250×4.6 mm). The flow rate was 20 ml/min and the LCMS split ratio 1/1000.

Other very suitable technologies for forming intramolecular disulfide bridges are disclosed in PCT/EP2006/012526, herein incorporated by reference.

III. In-Vitro Assays with Monomers
Proliferation Assay with TF-1 Cells by BrdU Incorporation

TF-1 Cells in logarithmic growth phase (˜2×10⁵-1×10⁶ cells/ml; RPMI medium; 20% fetal calf serum; supplemented with Penicillin, streptomycin; L-Glutamine; 0.5 ng/ml Interleukin 3) are washed (centrifuge 5 min. 1500 rpm and resuspend in RPMI complete without IL3 at 500,000 cells/ml) and precultured before start of the assay for 24 h without IL-3. At the next day the cells are seeded in 24- or 96-well plates usually using at least 6 concentrations and 4 wells per concentration containing at least 10,000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 3-day culture period. At the end of this culture period, FdU is added to a final concentration of 8 ng/ml culture medium and the culture continued for 6 hours. Then, BrdU (bromodeoxyuridine) and dCd (2-deoxycytidine) are added to their final concentrations (10 ng/ml BrdU; 8 ng/ml dCD; final concentrations in culture medium) and culture continued for additional 2 hours.

At the end of this incubation and culture period, the cells are washed once in phosphate buffered saline containing 1.5% BSA and resuspended in a minimal amount liquid. From this suspension, cells are added dropwise into 70% ethanol at −20° C. From here, cells are either incubated for 10 min. on ice and then analysed directly or can be stored at 4° C. prior to analysis.

Prior to analysis, cells are pelleted by centrifugation, the supernatant is discarded and the cells resuspended in a minimal amount of remaining fluid. The cells are then suspended and incubated for 10 min in 0.5 ml 2M HCl/0.5% triton X-100. Then, they are pelleted again and resuspended in a minimal amount of remaining fluid, which is diluted with 0.5 ml of 0.1N Na₂B₄O₇, pH 8.5 prior to immediate repelleting of the cells. Finally, the cells are resuspended in 40 μl of phosphate buffered saline (1.5% BSA) and divided into two reaction tubes containing 20 μl cell suspension each. 2 μl of anti-BrdU-FITC (DAKO, clone Bu20a) are added to one tube and 2 μl control mIgGl-FITC (Sigma) are added to the second tube starting an incubation period of 30 min. at room temperature. Then, 0.4 ml of phosphate buffered saline and 10 μg/ml Propidium Iodide (final concentration) are added. Analysis in the flow cytometer refers to the fraction of 4C cells or cells with higher ploidy and to the fraction of BrdU-positive cells, thus determining the fraction of cells in the relevant stages of the cell cycle.

Proliferation Assay with TF-1 Cells by MTT

TF-1 Cells in logarithmic growth phase (˜2×10⁵-1×10⁶ cells/ml; RPMI medium; 20% fetal calf serum; supplemented with Penicillin, streptomycin. L-Glutamine; 0.5 ng/ml Interleukin 3) are washed (centrifuge 5 min. 1500 rpm and resuspended in RPMI complete without IL3 at 500,000 cells/ml) and precultured before start of the assay for 24 h without IL-3. At the next day the cells are seeded in 24- or 96-well plates usually using at least 6 concentrations and 4 wells per concentration containing at least 10,000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 4-day culture period.

At day 4, prior to start of the analysis, a dilution series of a known number of TF-1 cells is prepared in a number of wells (0/2500/5000/10000/20000/50000 cells/well in 100 μl medium). These wells are treated in the same way as the test wells and later provide a calibration curve from which cell numbers can be determined. Having set up these reference wells, MTS and PMS from the MTT proliferation kit (Promega, CellTiter 96 Aqueous non-radioactive cell proliferation assay) are thawed in a 37° C. water bath and 100 μl of PMS solution are added to 2 ml of MTS solution. 20 μl of this mixture are added to each well of the assay plates and incubated at 37° C. for 3-4 h. 25 μl of 10% sodium dodecyl sulfate in water are added to each well prior to measurement E492 in an ELISA Reader.

### IV. Synthesis of Bivalent EPO Mimetic Peptide Units

The synthesis is carried out by the use of a Liberty microwave system (CEM) using Rink-Amide-Resin (substitution rate 0.19 mmol/g) in a scale of 0.25 mmol. Removal of Fmoc-groups is achieved by double treatment with 10 ml piperidine/DMF (1:3) and irradiation with 50 W for 10×10 sec. Coupling of amino acids is achieved by double treatment with a of 4 fold excess of amino acid in DMF PyBOP/HOBT/DIPEA as coupling additives and irradiation with 50 W for 5×30 sec. Between all irradiation cycles the solution is cooled by bubbling nitrogen through the reaction mixture. After deprotection and coupling, the resin is washed 6 times with 10 ml DMF. After the double coupling cycle all unreacted amino groups are blocked by treatment with a 10 fold excess of N-(2-Chlorobenzyloxycarbonyloxy)succinimide (0.2M solution in DMF) and irridation with 50 W for 3×30 sec. After deprotection of the last amino acid, the peptide is acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 m! TFA/TIS/EDT/H₂O (94/1/2.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

### Cyclization Reaction

30 mg of the linear peptide are dissolved in 60 ml solution A. This solution und 60 ml DMSO are added dropwise to 60 ml solution A (total time for addition: 3 h). After 48 h the solvents are removed by evaporation and the remaining residue solved in 30 ml DMSO/water (1/1). 30 ml acetic acid and 17 mg iodine (solved in DMSO/water (1/1) are added and the solution is mixed for 90 minutes at room temperature. Afterwards 20 mg ascorbic acid are added and the solvents removed by evaporation. The crude mixture is solved in acetonitrile/water (2/1) and the peptide is purified by RP-HPLC (Kromasil 100 C18 10 μm, 250×4.6 mm).

Solution A: Acetonitrile/water (1/1) containing 0.1% TFA. The pH is adjusted to 8.0 by the addition of ammonium bicarbonate.

The purification scheme: Purification of cyclic peptide, Kromasil 100 C18 10 μm, 250×4.6 mm, gradient from 5% to 35% acetonitrile (0.1% TFA) in 50 minutes.

### V. In Vitro Proliferation Assay to Determine EPO Activity

TF1 cells in logarithmic growth phase (2×10⁵-1×10⁶ cells/ml grown in RPMI with 20% fetal calf serum (FCS) and 0.5 ng/ml IL-3) were counted, and the number of cells needed to perform an assay were centrifuged (5 min. 1500 rpm) and resuspended in RPMI with 5% FCS without IL-3 at 300 000 cells/ml. Cells were precultured in this (starvation) medium without IL-3 for 48 hours. Before starting the assay the cells were counted again.

Shortly before starting the assay stock solutions of peptides and EPO were prepared. Peptides were weighed and dissolved in RPMI with 5% FCS up to a concentration of 1 mM, 467 μM or 200 μM. EPO stock solutions were 10 nM or 20 nM. 292 μl of these stock solutions were pipetted into one well of a 96 well culture plate—one plate was taken for each substance to be tested. Two hundred μl of RPMI with 5% FCS were pipetted into seventeen other wells in each plate. Ninety-two μl of stock solution were pipetted into a well containing 200 μl medium. The contents were mixed, and 92 μl from this well was transferred to the next, and so forth. This way a dilution series (18 dilutions) of each substance was prepared such that in each consecutive well the concentration was 1:√10 of the concentration in the well before that. From each well 3×50 μl was transferred to three empty wells. This way each concentration of substance was measured in quadruplicate. Note that the uppermost and lowermost line of wells of each plate was left void.

Pre-treated (starved) cells were centrifuged (5 min. 1500 rpm) and resuspended in RPMI with 5% FCS at a concentration of 200 000 cells per ml. Fifty μl of cell suspension (containing 10 000 cells) was added to each well. Note that due to the addition of the cells the final concentrations of the substances in the wells were half that of the original dilution range. Plates were incubated for 72 h at 37° C. in 5% CO₂.

Before starting the evaluation, a dilution range of known amounts of TF-1 cells into wells was prepared: 0/2500/5000/10000/20000/50000 cells/well were pipetted (in 100 μl RPMI+5% FCS) in quadruplicate.

To measure the number of live cells per well, ready-to-use MTT reagent (Promega, CellTiter 96 Aqueous One Solution Cell Proliferation Assay) was thawed in a 37° C. water bath. Per well, 20 μl of MTT reagent was added, and plates were incubated at 37° C. in 5% CO₂ for another 1-2 h. Twenty-five μl of a 10% SDS solution was added, and plates were measured in an ELISA reader (Genios, Tecan). Data were processed in spreadsheets (Excel) and plotted in Graphpad.

### VI. Extended Peptide Assays

In an extended assay, several peptide sequences were tested for their EPO mimetic activity.

The peptides were synthesized as peptides amides on a LIPS-Vario synthesizer system. The synthesis was performed in special MTP-synthesis Plates, the scale was 2 μmol per peptide. The synthesis followed the standard Fmoc-protocol using HOBT as activator reagent. The coupling steps were performed as 4 times coupling. Each coupling step took 25 min and the excess of amino acid per step was 2.8. The cleavage and deprotection of the peptides was done with a cleavage solution containing 90% TFA, 5% TIPS, 2.5% H₂O and 2.5% DDT. The synthesis plate containing the finished peptide attached to the resin was stored on top of a 96 deep well plate. 50 μl of the cleavage solution was added to each well and the cleavage was performed for 10 min, this procedure was repeated three times. The cleaved peptide was eluted with 200 μl cleavage solution by gravity flow into the deep well plate. The deprotection of the side chain function was performed for another 2.5 h within the deep well plate. Afterwards the peptide was precipitated with ice cold ether/hexane and centrifuged. The peptides were solved in neutral aqueous solution and the cyclization was incubated over night at 4° C. The peptides were lyophilized.

FIG. 7 gives an overview over some of the synthesised and tested peptides monomers.

The peptides were tested for their EPO mimetic activity in an in vitro proliferation assay. The assay was performed as described under V. On each assay day, 40 microtiter plates were prepared for measuring in vitro activity of 38 test peptides, 1 reference example, and EPO in parallel. EPO stocks solutions were 20 nM.

### VII. Synthesis of Peptide HES-Conjugates

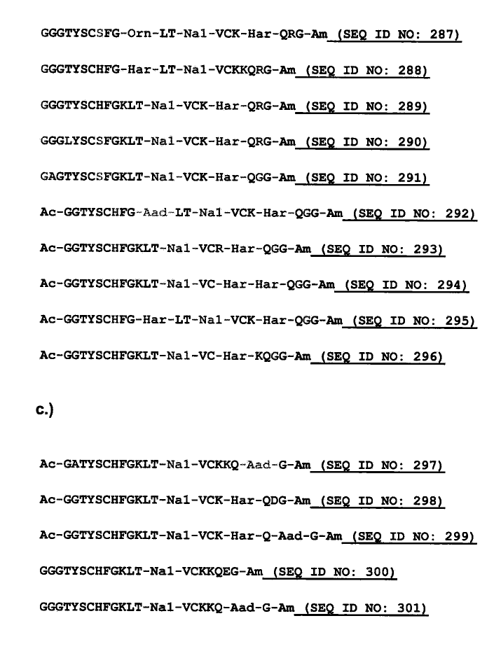

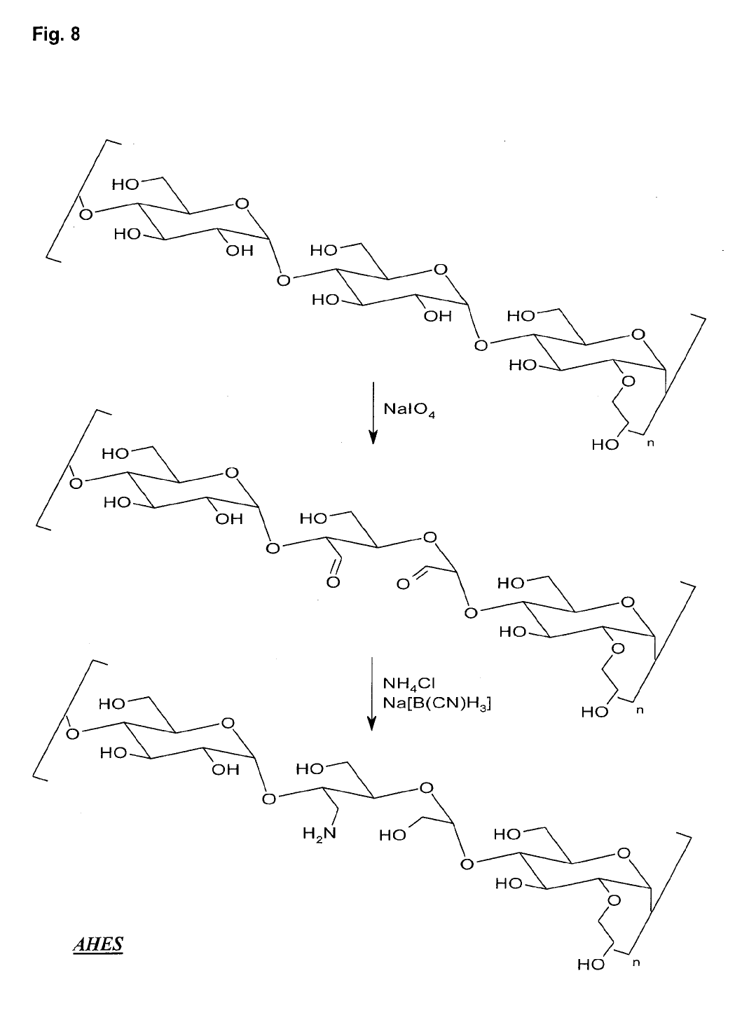

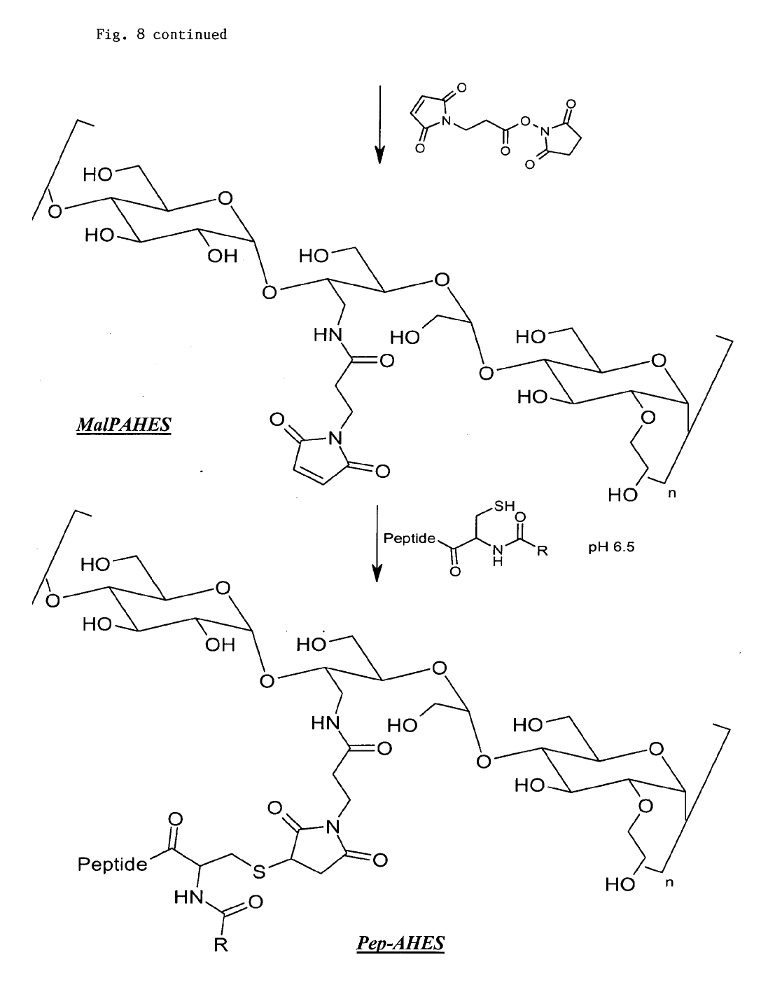

The principle reaction scheme is depicted in FIG. 8. Alternative strategies for coupling dimeric peptides to the carrier are disclosed in WO 2006/136450, herein incorporated by reference.

The aim of the described method is the production of a derivative of a starch, according to this example HES, which selectively reacts with thiol groups under mild, aqueous reaction conditions. This selectivity is reached with maleimide groups.

HES is functionalised first with amino groups and converted afterwards to the respective maleimide derivative. The reaction batches were freed from low molecular reactants via ultra membranes. The product, the intermediate products as well as the educts are all poly-disperse.

### Synthesis of Amino-HES (AHES)

Hydroxyethylstarch (i.e. HES 130/0.4 or HES 200/0.5) was attained via diafiltration and subsequent freeze-drying. The average molar weight was approximately 130 kD with a molar degree of substitution of 0.4, respectively 200 kD, MS=0.5.

The synthesis was performed according to the synthesis described for amino dextran in the dissertation of Jacob Piehler, “Modifizierung von Oberflächen für die thermodynamische und kinetische Charakterisierung biomolekularer Erkennung mit optischen Transducern”, 1997, herein incorporated by reference. HES was activated by partial, selective oxidation of the diolic hydroxyl groups to aldehyde groups with sodium periodate as described in Floor et. al (1989). The aldehyde groups were converted via reductive amination with sodiumcyanoborhydride (Na[B(CN)H₃]) in the presence of ammonia to amino groups (Yalpani and Brooks, 1995).

### Periodate Opening

By a mild oxidation of the 1,2-diols in the saccharide by sodium periodate in water aldehyde groups are introduced. By using different molar concentration of the oxidizing agent the number of available anchor groups and so the amount of peptide drug on the carrier can be controlled. To optimize the protocol the oxidation was monitored with the reagent Purpald that forms a purple adduct only with aldehydes. The reaction time can be reduced down to 8-18 h. The used amount of periodate represents 20% of the number of glucose building blocks (applying a glucose building block mass of 180 g/mol, DS=0.4). The working-up was performed via ultra filtration and freeze-drying. The purification of each polymeric product was performed by ultrafiltration techniques using a PES membrane of different molecular weight cut offs followed by lyophilisation. From the optimized HES derivatives only the molar mass range larger than 100 kD were used.

### Aldehyde Analysis

Qualitative/Semi-quantitative: Purpald reaction of the available aldehyde groups.

Reductive Animation with Ammonium Chloride

In the following step the introduced aldehyde groups were converted into amines by a reductive amination in a saturated solution of ammonium chloride at a slightly acidic pH value with sodium cyanoborohydride.

To optimize the protocol the aldehyde groups of the starting material were followed by the Purpald reagent and the formed amines with TNBS. These experiments have shown that the formation of the imine intermediate is in an equilibrium after a starting period and the added reducing agent prefers the imins over the aldehyde. So could be found that the optimal reaction is performed by several addition of the reducing agent with a total reaction time of 24 h.

Working-up via precipitation of the product and dia- or ultrafiltration.

### Amine Analysis

Qualitative: Ninhydrin reaction (Kaiser-test)
Semi-quantitative: with 2,4,6-trinitrobenzole sulphonic acid (TNBS) in comparison with an amino dextrane.

The achieved substitution grade was around 2.8%. This results in a molar mass of one building block carrying one amino group of approx. 6400 g/mol.

Synthesis of maleimidopropionyl-amino-hydroxyethylstarch (“MaIPA-HES”)

After introduction of amino groups the anchor maleimide groups are introduced with ω-maleimido alkyl (or aryl) acid-N-hydroxysuccinimidesters.

### Synthesis

The final introduction of the maleimide groups into the HES is performed with 3-maleimidopropione acid-N-hydroxysuccinimidester (MaIPA-OSu). When using an excess (5 to 10-fold) in a slightly acidic buffer the conversion is quantitavely (50 mM phosphate buffer, pH 7, 20% DMF, over night). The ultrafiltrated and lyophilized product is stored at −18° C.

### Analysis

The reaction of the amino group was verified with ninhydrin and TNBS. The number of introduced maleimide groups is demonstrated by reaction of glutathione (GSH) and the detection of excessive thiol groups with Ellmans reagent 6,6′-dinitro-3,3′-dithiodibenzoic acid (DTNB) and via 700 MHz-¹H-NMR-spectroscopy

The achieved substitution grade was around 2% and corresponds to 8500 g/mol per maleimide building block (180 g/mol glucose building block mass, MS=0.4).

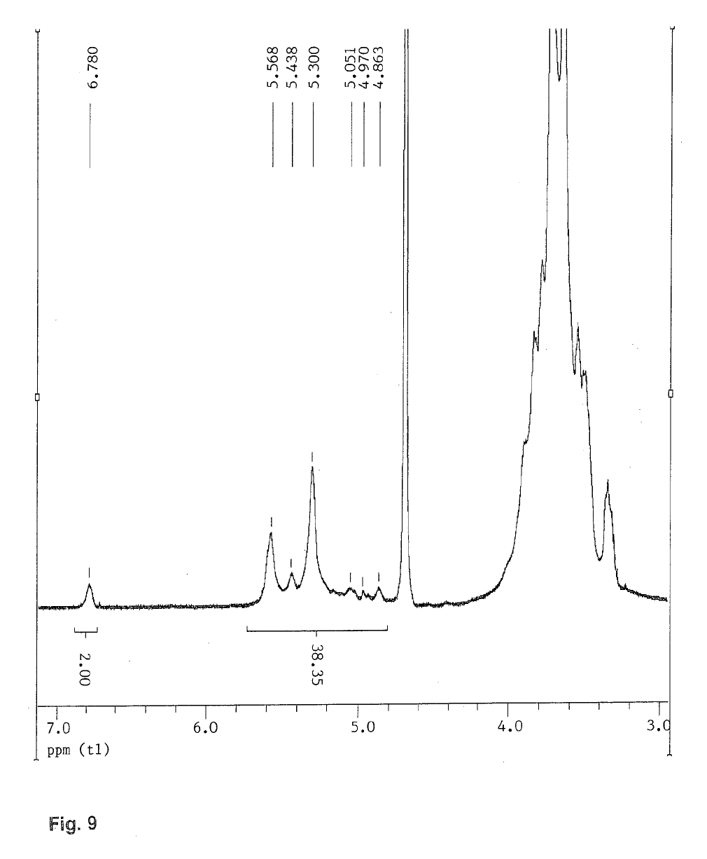

FIG. 9 shows a ¹H-NMR spectra (D₂O, 700 MHz) of a maleimide modified HES. Ratio of the maleimide proton (6.8 ppm) to the anomeric C—H (4.8-5.6 ppm) gives a building block size of approx. 6,900 g/mol (in comparison: the GSH/DTNB test gave 7,300 g/mol).

The number of maleimide groups and so the building block size can be measured by saturation with GSH and reaction with DTNB. The formed yellow colour is significant and can be quantified easily. These values give reliable building block sizes in between 5,000 and 100,000 g/mol depending on the used starting material, respectively the amount of periodate in the oxidation step. This method has been validated by ¹H-NMR spectroscopy of the product. In the NMR the content of maleimide groups can be quantified from the ratio of all anomeric C—H signals and the maleimide ring protons.

The following ranges are preferred:

TABLE 1
Examples for the reachable virtual building block size of the
anchor group in the HES backbone via the periodate oxidation.

| Amount of periodate (1^{st} | |
|---|---|
| step) (eq) | Building block sizes maleimide (g/mol) |
| 0.01-0.03 | >55,000 |
| 0.02-0.04 | Approx. 35,000-50,000 |
| 0.04-0.1 | Approx. 15,000-35,000 |
| 0.1-0.3 | Approx. 6,000-7,000 |

### Peptide-hydroxyethylstarch-conjugate (Pep-AHES)

### Synthesis

A cysteine containing peptide was used which had either a free (Pep-IA) or a biotinytated (Pep-IB) N-term. A 4:1 mixture of Pep-IA/B was converted over night in excess (approx. 6 equivalents with MaIPA-HES in phosphate-buffer, 50 mM, pH 6.5/DMF 80:20; working up occurred with ultra filtration and freeze-drying.

### Analysis

The UV-absorption was determined at 280 nm and the remaining content of maleimide groups was determined with GSH/DTNB.

The peptide yield was almost quantitative. Nearly no free maleimide groups were detectable.

For the conjugation of the peptide drug a peptide domain

Ac-GGTYSCHFGKLT-Na1-VCKKQRG-Am (BB68)

is used for creating a peptide unit by introducing a free thiol group (e.g. by introducing a cysteine residue at the N-terminus) as in

Ac-C(tBu)-GGTYSCHFGKLT-Na1-VCKKQRG-GGTYSCHFGKLT-
Na1-VCKKQRG-Am (AGEM40)

an 10-50% excess of the deprotected peptide is conjugated in a slightly acidic buffer for 1-2 h. The conditions have been optimized to assure on the one hand that the HES backbone, the maleimide groups and the disulfide bridges are stable and on the other hand to observe a quantitative conversion. By using different maleimide functionalized HES compounds a number of supravalent EPO-Mimetic Peptides were synthesised, which have shown in vitro a supravalent effect. Some examples are given below

TABLE 2
Supravalent EPO-mimetic Peptide conjugates
of AGEM40 with different peptide contents.

| | | Peptide | Peptide |
|---|---|---|---|
| Supravalent EPO- | Building block | content | content |
| Mimetic Peptide | sizes maleimide | theoretical | experimental |
| on HES | groups (g/mol) | (%) | (%) |
| AGEM40-HES A2 | 7,300 | 39 | 37 |
| AGEM40-HES A3 | 16,000 | 23 | 22 |
| AGEM40-HES A4 | 44,000 | 10 | 10 |

An easy chemical analysis of the supravalent EPO-mimetic peptide conjugates was realized in two steps. First the content of peptide was quantified by HPLC after a soft hydrolysis of the HES backbone and second the amount of polysaccharide was measured by a calorimetric test with phenol after a complete hydrolysis by sulphuric acid.

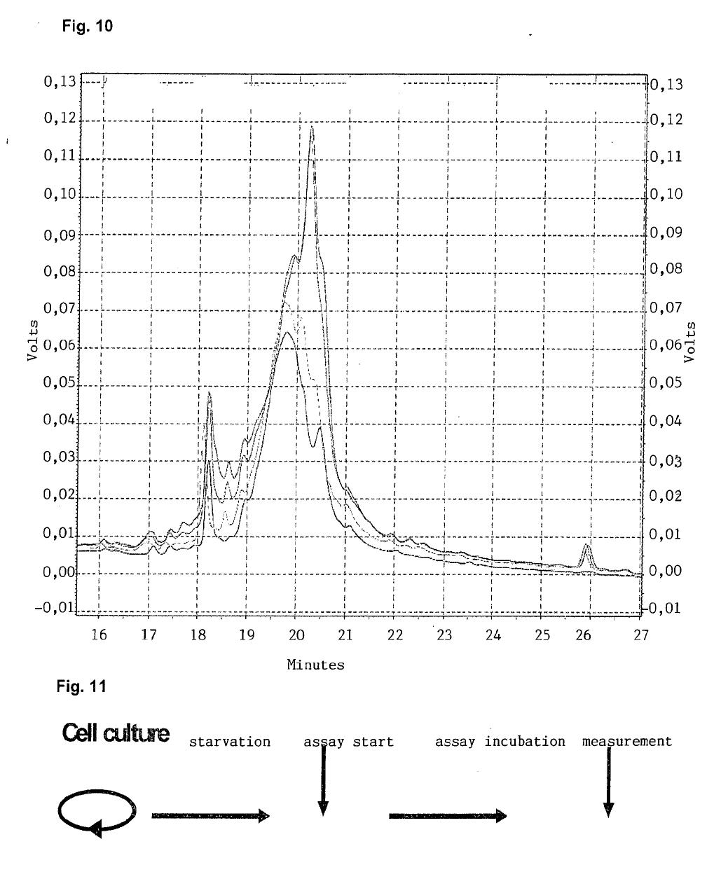

FIG. 10 shows a HPLC chromatogram (Shimadzu HPLC) of the TFA/water hydrolysis of the Supravalent EPO-Mimetic Peptide-conjugates AGEM40-AHES A2. After a certain time the UV absorbance of all peptide containing species is constant at a maximal value and by comparison with the free peptide a peptide content of 37% can be calculated (theoretical value: 39%).

### VIII. Further In Vitro Experiments

Many of the experiments described below were already described above. However, the following details give a summarising overview over the described tests and results. Predominantly the human cell culture and bone marrow assays are discussed.

On one hand, rapid cell-line based assays were used to check for potency of optimised peptide sequences throughout the early stages of optimisation. These cell culture assays are still valid as rapid tests of efficacy of a new peptide or a new batch. The two endpoints, which were used for the cell line TF-1 (human cells) are proliferation (here usually determined as number of living cells at defined time points) and differentiation as marked haemoglobin production in TF-1 cells.

In addition, primary cells (human bone marrow stem cells) were used for CFU-assays, which are very close to the in vivo situation. They give answers to erythropoietic activity in case of the use of EPO mimetic peptides as peptide units in a much more in vivo-like fashion. However, they are to be handled more sophisticated and need more time per assay than the cell culture assays.

### Assays Using Human TF-1 Cells

TF-1 is a human erythroleukemia cell line that proliferates only in response to certain cytokines such as IL3 or EPO. In addition, TF-1 cells can differentiate towards an erythroid phenotype in response to EPO. TF-1 cells were obtained from DSMZ (Braunschweig, Germany). A product sheet is available at the DSMZ web site dsmz.de. TF-1 is the cell line recommended for EPO-activity assessment by the European Pharmacopoe.

Our internal culture protocol for maintenance culture:

Medium: RPMI+P/S+AmphoB+L-Glut.+20% FCS+h-IL-3
1. −500 ml RPMI+5 ml P/S+5 ml AmphoB
2. −200 ml RPMI+PS/AmphoB+2.5 ml L-Glutamine+50 ml FCS=complete Medium (1 month 4° C.)
3. −45 ml complete Medium+22.5 ul h-IL-3 (1 week 4° C.)
   Culture: Maintain between 200,000 and 1,000,000 cells/ml For 3 days 2×10⁵/ml
For 2 days 3×10⁵/ml
   For 1 days 5×10⁵/ml

### Design of a TF-1 Proliferation Assay

In a TF-1 proliferation assay, TF-1 cells are seeded and cultured for several days in varying concentrations of EPO or EPO mimetic peptides in a multi-well plate.

For optimal results TF-1 cells should be cultured for two days in the absence of any cytokine (starved) before starting the assay. Three days after starting the assay, cell proliferation is measured indirectly by assaying the number of viable cells.

A tetrazolium reagent, called MTS, is added which is reduced to coloured formazan. This reaction depends on NADH and NADPH, in other words depends on mitochondrial activity. The amount of formazan is measured spectrophotometrically. Using a range of known cell numbers for calibration, it is possible to determine the absolute number of viable cells present under each condition. The principal design is also illustrated in FIG. 11.

The activity of a certain agent in this assay is determined by:
1. assessing whether this agent causes an increase in the number of viable cells at a certain concentration, and
2. at which concentration this agent exerts a half-maximum effect (determination of the EC50).
   Results of TF-1 Proliferation Assays As a general remark, it has to be mentioned that all EPO-mimetic peptides (EMP1 and the modified peptides described above) behave in their monomeric form in this assay as partial agonists, i.e. the maximal response is weaker than the response seen with EPO. Nevertheless, the assay can be used to determine the right/left shift in normalized plots and thus to determine the outcome of optimisations. This especially, as it is known that the agonist activity considerably increases upon dimerisation.

The first graph depicts this effect in absolute response without normalisation. All other graphs show normalized plots, which allow determination of EC50 values from the curves.

Two reference substances were used in the assays:
1) EMP1, a published peptide sequence with known EPO-mimetic properties (Johnson et al, 1997).
2) Recombinant Human Erythropoietin (EPO), was bought in the pharmacy as the Ortho Biotech product Epoetin alfa (Tradename in Germany: Erypo^{R})

The plots of these substances are given as black lines, continuous for EPO and dotted for EMP1

The proline-modified EPO mimetic peptides are shown in the next Figs. as coloured continuous lines. These modified peptides depict the following sequence:
1) BB49

Ac-GGTYSCHFGKLTWVCKKQGG

shows an efficacy and potency in the same range as EMP1
2) BB68

Ac-GGTYSCHFGKLT-Na1-VCKKQRG-Am

is even more effective than EMP1 and BB49
3) AGEM40,

Ac-C(tBu)-GGTYSCHFGKLT-Na1-VCKKQRG-GGTYSCHFGKLT-
Na1-VCKKQRG-Am

which is a bivalent continuous peptide, which was designed based on the sequence of BB68 depicting improved features.
4) AGEM40—HES, which is an advanced, highly effective and potent peptide (AGEM40) HESylated according to the supravalence principle of the present invention.

These sequences were used as examples inter alia in order to illustrate the benefits of the supravalence principle.

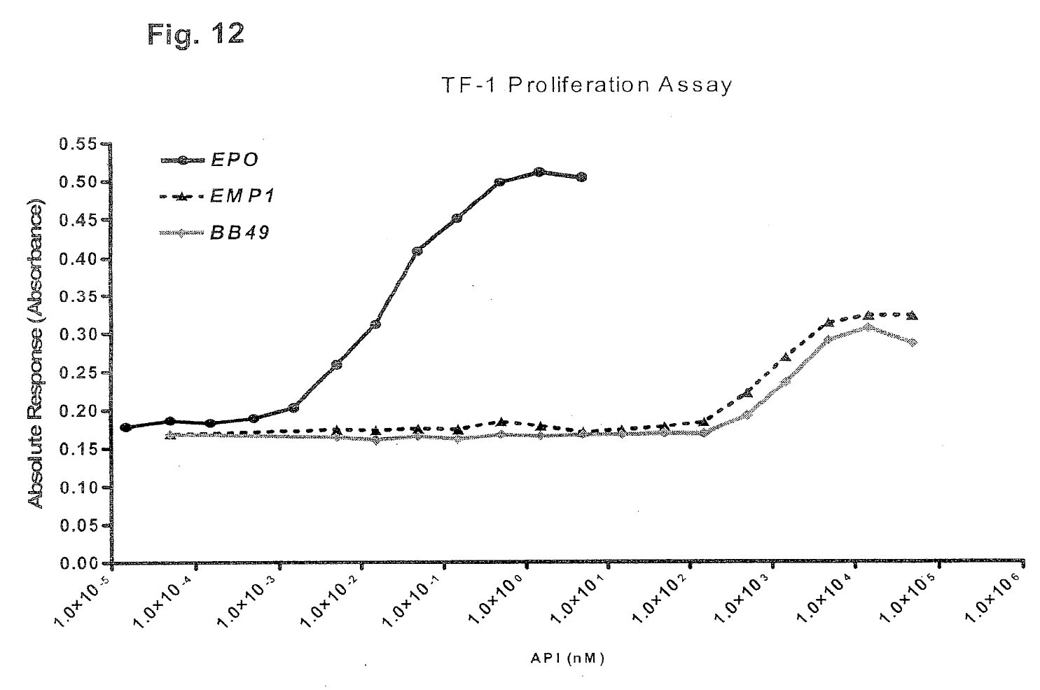

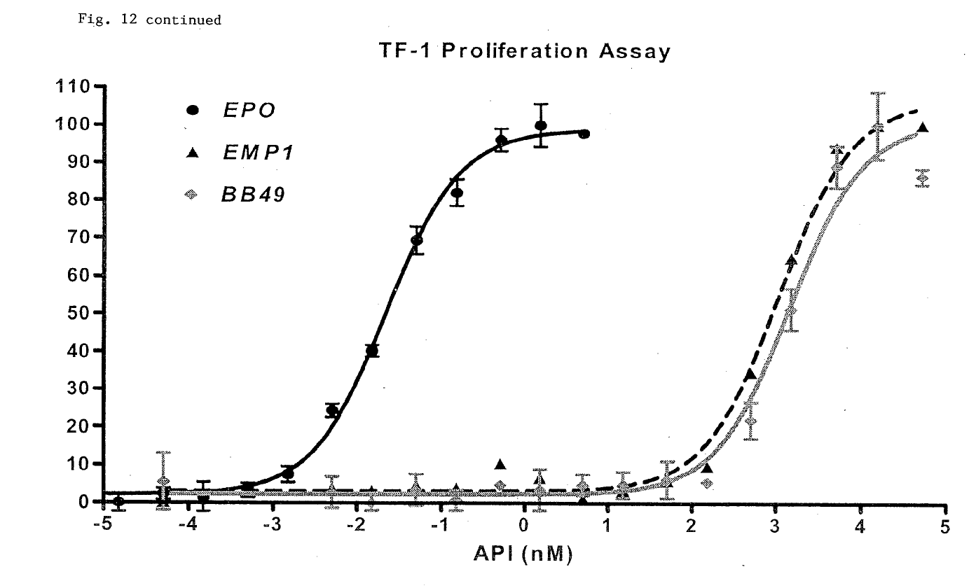

FIG. 12 describes the results of monomeric EPO mimetic peptides in comparison with EPO. FIG. 12 includes a plot of actual absorbance data documenting the absolute difference between peptides in general and EPO in this assay.

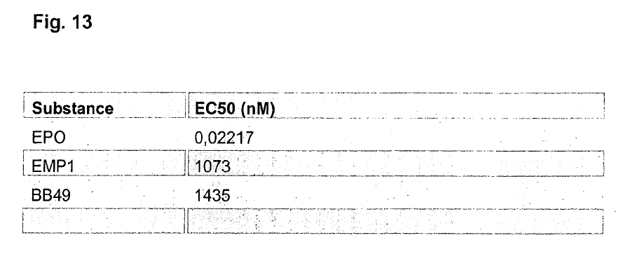

FIG. 13 gives the EC50 values calculated from the fitted normalized plots.

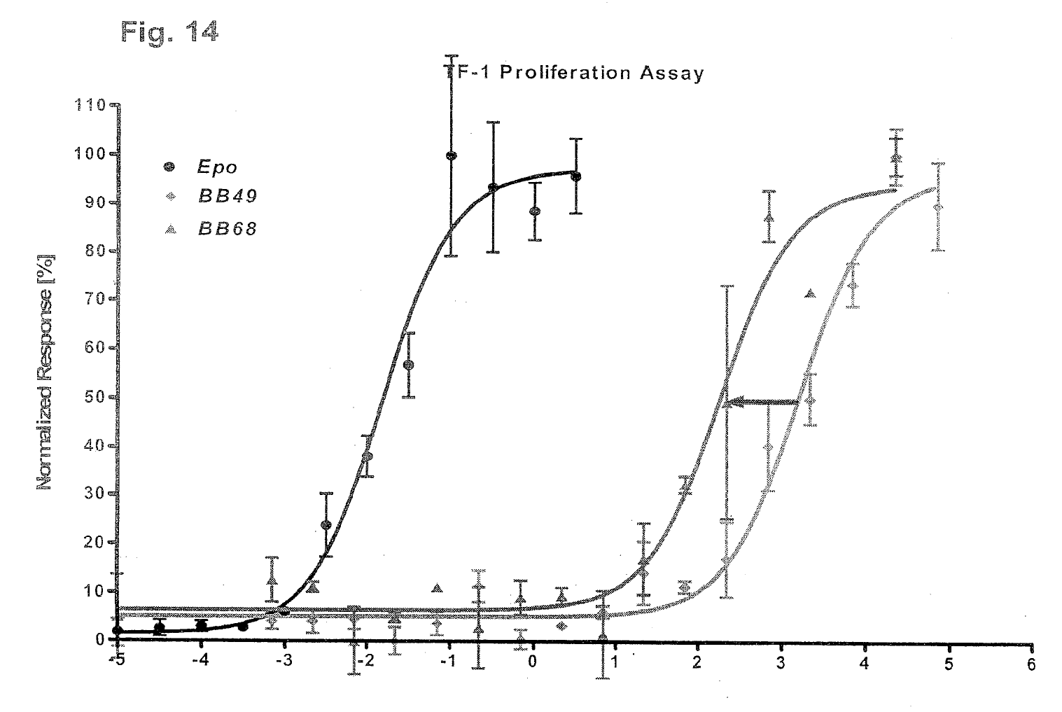

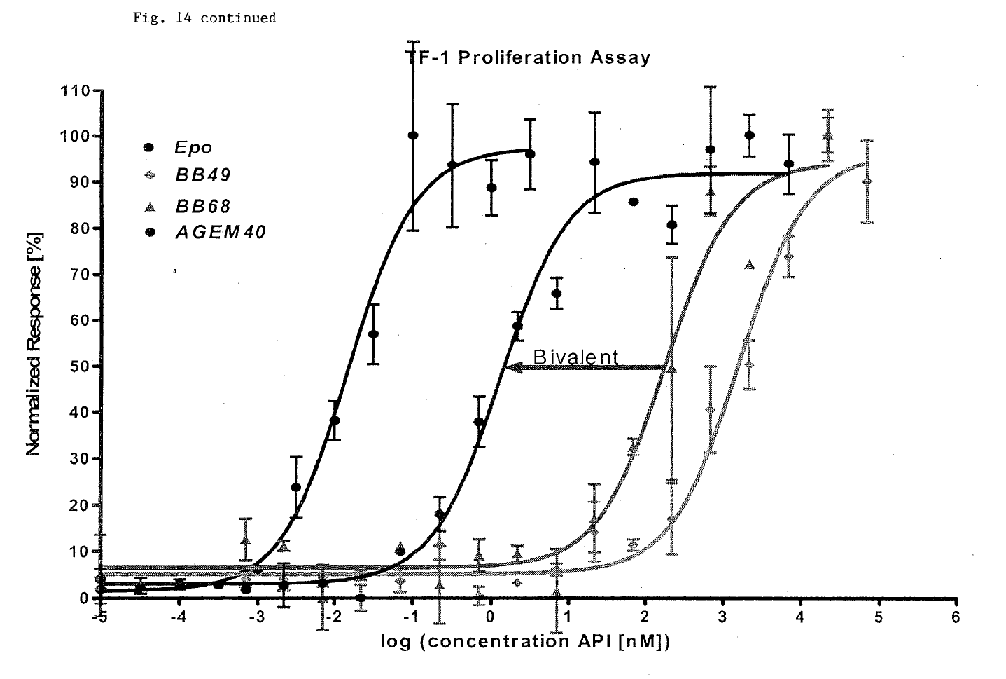

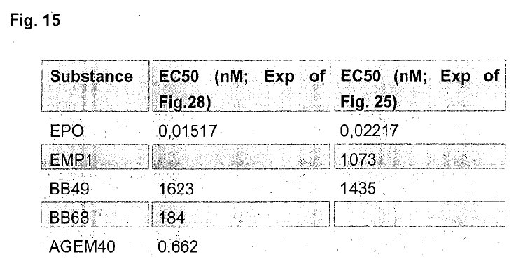

FIG. 14 shows the improved effect of BB68 compared to BB49. Using the optimized BB68 as building block for creating a peptide unit according to the present invention, the effect was improved by two additional orders of magnitude. This is documented in FIG. 14 and the corresponding Table shown in FIG. 15.

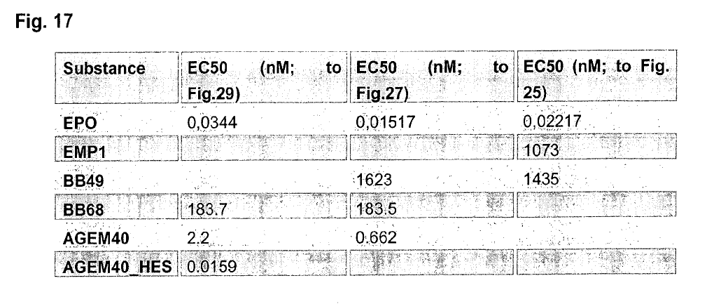

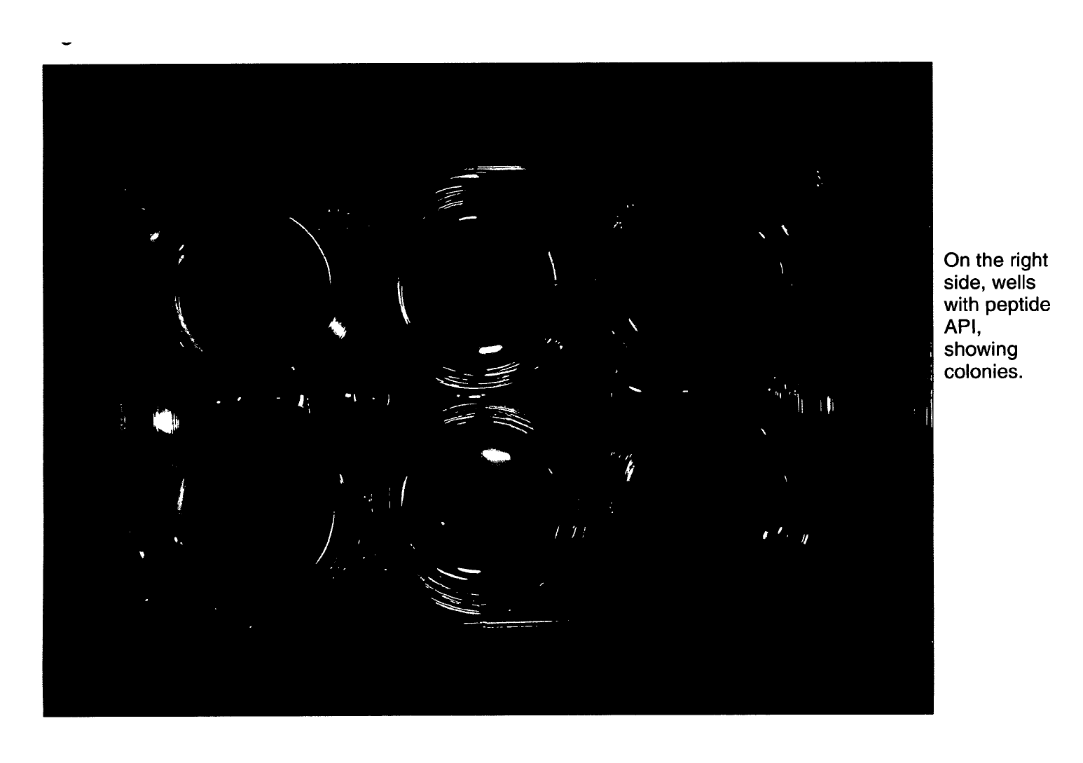

The dimeric peptide units were then coupled to the macromolecular carrier HES at an optimized density. The resulting API is at least equipotent to EPO on molar comparison and very close to EPO on mass comparison (see FIG. 16 and FIG. 17 below).

FIG. 16 and the Figures and Tables before clearly demonstrate the great potency of the supravalence concept. Keeping the accuracy in mind, which can be achieved with a cell culture assay, the achieved API is at least equipotent to EPO in vitro. It is thus superior to any known EPO-mimetic peptide API not employing the supravalence concept.

### Bone Marrow Assays

Bone marrow contains hematopoietic stem cells with a potential so self-renew and to develop into all types of blood cells. In addition, bone marrow contains committed progenitor cells capable of developing into one or several blood cell lineages. Among those progenitor cells, some develop into erythrocytes (erythroid progenitors).

Progenitor cells can be demonstrated by plating bone marrow cells in methylcellulose-based semi-solid media. In the presence of an appropriate cytokine cocktail progenitor cells proliferate and differentiate to yield a colony of cells of a certain lineage. Myeloid progenitors develop into granulocytic colonies (derived from a CFU-G), monocytic colonies (from a CFU-M), or mixed granulocytic-monocytic colonies (from a CFU-GM). Erythroid progenitors develop into a colony of erythrocytes (red cells). Depending on the size of the erythroid colony, the progenitor cells are called BFU-E (yielding colonies of 200 cells or more) of CFU-E (yielding colonies of less than 200 cells). Progenitor cells in an earlier stage of commitment can develop into mixed granulocytic-erythroid-monocytic-megakaryocytic colonies. These early progenitors are called CFU-GEMM.

EPO stimulates the development of erythroid colonies from BFU-E or CFU-E, if certain different cytokines are present as well. Without EPO no erythroid colonies can develop. Outgrowth of erythroid colonies from a homogenous batch of bone marrow cells in methylcellulose, therefore, is a measure for EPO activity.

Since the abovementioned processes are very similar if not identical to the processes which occur in the bone marrow in vivo, they are an excellent predictor of EPO-like activity.

### Design of Bone Marrow Assays

Human bone marrow cells (commercially available from Cryosystems, serologically checked) are thawed from cryovials, and plated in methylcellulose media with a given background of cytokines (but without EPO) at a fixed cell density. EPO or EPO-mimetic peptide is added at varying concentrations.

Cultures are incubated for 12-14 days at 37 C. Then, the numbers of myeloid and erythroid colonies are enumerated by microscopic inspection.

### End Points of Bone Marrow Assays:

1. Premisses: Cultures without EPO should only yield myeloid (white) but not erythroid (red) colonies. Cultures with EPO should yield a concentration-dependent increase in red cell colonies, and a concentration-dependent increase in the sizes of the red cell colonies.
2. A peptide shows EPO-mimetic activity if it causes a concentration-dependent increase in red cell colonies, and a concentration-dependent increase in the sizes of the red cell colonies. However, a peptide should not interfere with the numbers of myeloid colonies obtained.

### Results of Bone Marrow Assays

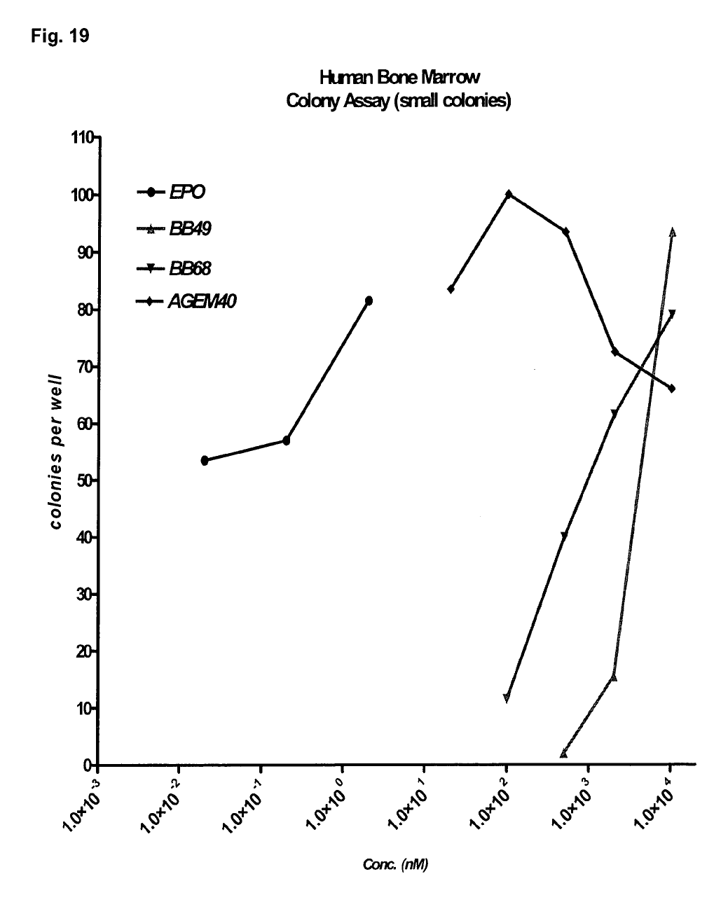

The proline modified EPO mimetic peptides described above did not stimulate the formation of myeloid colonies, but showed significant activity on the formation of red colonies. Qualitatively, this is shown in the FIG. 18 in a photograph of a culture plate, while counting of colonies is documented in FIG. 19.

### IX. Antibody-Cross Reactivity Assay

As described in the introduction of this application, patients sometimes develop antibodies against rhuEPO. This leads to the severe consequences described in the introduction.

In order to further explore the properties of the peptides according to the invention it was analysed whether the peptides in fact cross-react with anti-EPO antibodies.

Rabbit and human sera containing anti-EPO antibodies were used for testing. These sera were pre-treated either with EPO or the following EPO mimetic peptides:

Ac-C-GGTYSCHFGKLT-1nal-VCKKQRG-GGTYSCHFGKLT-1nal-
VCKKQRG-Am(test peptide 1)
Ac-GGTYSCHFGKLT-1nal-VCKKQRG-Am (test peptide 2)

Ac=acetylated N-terminus
Am=amidated C-terminus
1nal=1-naphthylalanine

Different concentrations of erythropoietin and EPO mimetic peptides were used in the analysis. After pre-treatment of the sera with the test substances in order to adsorb the anti-EPO antibodies present in the sera, the sera were treated with radioactively labelled erythropoietin. The antibodies remaining in the sera after the pre-adsorption step are bound by the erythropoietin and again immunoprecipated. The protocol used for this test is described in Tacey et al., 2003, herein incorporated by reference.

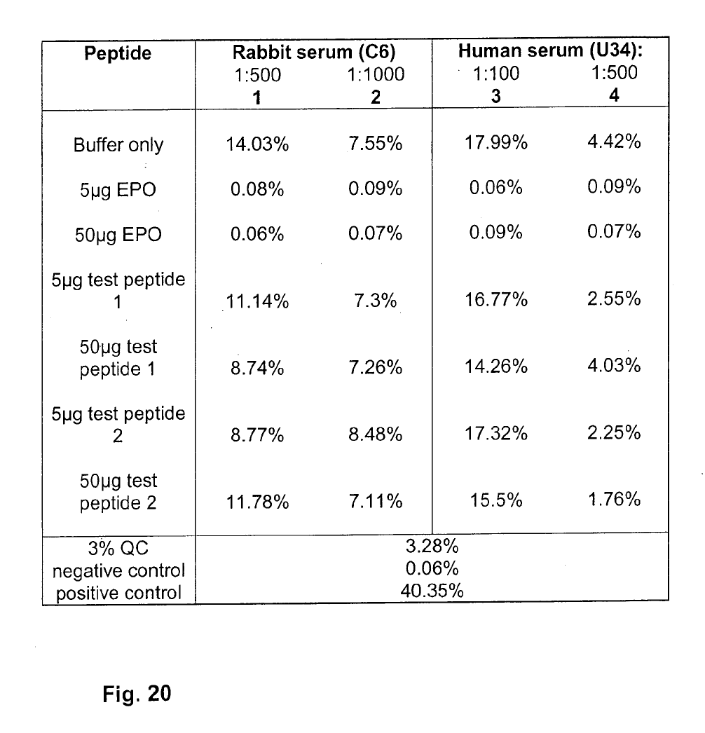

The results of the performed pre-adsorption with the anti-EPO antibody containing sera using either EPO or EPO mimetic peptides according to the invention are disclosed in FIG. 20.

When the sera were pre-treated with EPO mimetic peptides, the sera were afterwards tested positive when contacted with radioactively labelled erythropoietin. Thus anti-EPO antibodies were detected in the sera notwithstanding the pre-treatment. This means that the EPO mimetic peptides were not able to bind to the anti-EPO antibodies during pre-treatment. In the absence of a binding activity, the anti-EPO antibodies were not eliminated from the sera together with the EPO mimetic peptides and thus remained in the sera. The anti-EPO antibodies were not able to recognize and thus bind to the EPO mimetic peptides.

Recombinant human EPO (rhuEPO) was used as a control. When the sera were pre-treated with erythropoietin, pretty much no antibodies were detectable in the subsequent assay incorporating radioactively labelled erythropoietin since the antibodies were already bound and eliminated by the pre-treatment with erythropoietin.

The numerical values depicted in FIG. 20 represent the % cpm of the total counts used in the IP. A serum is assessed as positive when the % cpm value is >0.9. 100% cpm represents the amount of the overall used counts (the radioactive tracer), presently the radioactively labelled EPO.

The assay demonstrates that the EPO mimetic peptides according to the invention depict advantageously no cross-reactivity to anti-EPO antibodies. The EPO mimetic peptides described herein should thus depict a therapeutic effect even in patients who developed antibodies against rhuEPO. Furthermore, it is expected, that antibodies against EPO mimetic peptides should not bind erythropoietin. The EPO mimetic peptides according to this invention are thus preferably also characterised in that they show no significant cross-reactivity with anti-EPO antibodies.

### X. Efficacy in Primates

The efficacy of the EPO mimetic peptides according to the present invention was also proved in animal studies, wherein 7 non-naive monkeys (macaca fascicularis) were used for testing. The test peptide was AGEM 400 HES (see above) which was used as a lyophilised powder, solved in Ringer Solution. Doses between 0.01 mg/kg and 50 mg/kg were tested (intravenous administration). The animal experiments showed that the EPO mimetic peptide depicts a good EPO mimetic efficacy even at low doses and had a long-lasting effect. Also, no signs of toxicity were observed.

### REFERENCES

Wrighton N C, Balasubramanian P, Barbone F P, Kashyap A K, Farrell F X, Jolliffe L, Barrett R W, Dower W J (1997) Increased potency of an erythropoietin peptide mimetic through covalent dimerization. Nature Biotechnology 15:1261-1265
Wrighton N C, Farrell F X, Chang R, Kashyap A K, Barbone F P, Mulcahy L S, Johnson D L, Barrett R W, Jolliffe L K, Dower W J (1996) Small Peptides as Potent Mimetics of the Protein Hormone Erythropoietin. Science 273:458-463
Johnson, D. L., F. X. Farrell, et al. (1997). “Amino-terminal dimerization of an erythropoietin mimetic peptide results in increased erythropoietic activity.” Chemistry and Biology 4: 939-950.
Johnson, D. L., F. X. Farrell, et al. (1998). “Identification of a 13 Amino Acid Peptide Mimetic of Erythropoietin and Description of Amino Acids Critical for the Mimetic Activity of EMP1”. Biochemistry 37, 3699-3710.
Haag R, Sunder A, Stumbé J F, J. Am. Chem. Soc. (2000), 122, 2954.
Roberts, M. J., M. D. Bentley, et al. (2002). “Chemistry for peptide and protein PEGylation.” Advanced Drug Delivery Review 54(4): 459-476.
Richard Tacey, Anthony Greway, Janice Smiell, David Power, Arno Kromming a, Mohamed Daha, Nicole Casadevall and Marian Kelley: The detection of anti-erythropoietin antibodies in human serum and plasma—Part I. Validation of the protocol for a radioimmunoprecipitation assay; J Immunol Methods. 2003 December; 283(1-2):317-29.
Zalipsky S, Qazen, S, Walker II J A, Mullah N, Quinn Y P, (1999) “New detachable poly(ethylene glycol) conjugates: Cysteine-cleavable lipopolymers regenerating natural phospholipid, diacyl phosphatidylethanolamine, Bioconjug. Chem. 10: 703-707.
Zhao, X. et al (1997), “Novel Degradable Poly(ethylene glycol) esters for drug delivery.” In “Poly(ethylene glycol) chemistry and biological applications; Harris J M, Zalipsky, S. Eds.; ACS Symposium Series 680; American Chemical Society: Washington D.C., 1997; 458-472.

1. A peptide being capable of binding the EPO receptor, selected from:
peptides comprising the following consensus sequence of amino acids:

| | |
|---|---|
| X₆X₇X₈X₉X₁₀X₁₁X₁₂X₁₃X₁₄X₁₅ | (SEQ ID NO: 1) |

wherein each amino acid is selected from natural or unnatural amino acids and
X₆ is an amino acid with a sidechain functionality capable of forming a covalent bond or A or α-amino-γ-bromobutyric acid;
X₇ is R, H, L, W, Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is an uncharged polar amino acid or A;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid; and
functionally equivalent fragments, derivatives and variants of the peptides defined by the above consensus sequence, that depict an EPO mimetic activity and have an amino acid in position X₁₀ that constitutes a non-conservative exchange of proline or wherein X₉ and X₁₀ are substituted by a single amino acid.

2-5. (canceled)

6. A peptide of at least 10 amino acids in length, capable of binding to the EPO receptor and comprising an agonist activity, selected from:
(a) a peptide comprising the following core sequence of amino acids:

| | |
|---|---|
| X₉X₁₀X₁₁X₁₂X₁₃ | (SEQ ID NO: 2) |

wherein each amino acid is selected from natural or non-natural amino acids, and wherein:
X₉ is G or a conservative exchange of G;
X₁₀ is a non conservative exchange of proline or X₉ and X₁₀ are substituted by a single amino acid;
X₁₁ is selected from any amino acid;
X₁₂ is an uncharged polar amino acid or A;
X₁₃ is naphthylalanine:
(b) a peptide, especially one being capable of binding the EPO receptor comprising the following sequence of amino acids:

| | |
|---|---|
| X₆X₇X₈X₉X₁₀X₁₁X₁₂X₁₃X₁₄X₁₅ | (SEQ ID NO: 306) |

wherein each amino acid is selected from natural or unnatural amino acids and
X₆ is C, A, E, α-amino-γ-bromobutyric acid or homocysteine (hoc);
X₇ is R, H, L, W or Y or R, H, L, W, Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is T or A;
X₁₃ is 1-nal, 2-nal
X₁₄ is D, E, I, L or V;
X₁₅ is C, A, K, α-amino-γ-bromobutyric acid or homocysteine (hoc) provided that either X₆ or X₁₅ is C or hoc; and
(c) functionally equivalent fragments, derivatives and variants of the peptides defined by the above consensus sequences that depict an EPO mimetic activity and have an amino acid in position X₁₀ that constitutes a non-conservative exchange of proline or wherein X₉ and X₁₀ are substituted by a single amino acid and a naphthylalanine in position X₁₃.

7. A peptide according to claim 6, comprising the following core sequence of amino acids:

| | |
|---|---|
| X₆X₇X₈X₉X₁₀X₁₁X₁₂X₁₃X₁₄X₁₅ | (SEQ ID NO: 354) |

wherein each amino acid is selected from natural or non-natural amino acids, and wherein:
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
X₁₀ is a non conservative exchange of proline or X₉ and X₁₀ are substituted by a single amino acid;
X₁₁ is selected from any amino acid;
X₁₂ is an uncharged polar amino acid or A; preferably threonine, serine, asparagine or glutamine;
X₁₃ is naphthylalanine;
X₁₄ is D, E, I, L or V; and
X₁₅ is an amino acid with a sidechain functionality capable of forming a covalent bond or A or α-amino-γ-bromobutyric acid.

8. (canceled)

9. A peptide according to claim, further comprising the following additional amino acids positions:

| | |
|---|---|
| X₁₆X₁₇X₁₈X₁₉ | (SEQ ID NO: 355) |

wherein each amino acid is selected from natural or unnatural amino acids and
X₁₆ is independently selected from any amino acid;
X₁₇ is independently selected from any amino acid;
X₁₈ is independently selected from any amino acid; and
X₁₉ is independently selected from any amino acid.

10-11. (canceled)

12. A peptide according to claim 7, wherein a charged amino acid is present in position X₁₀, X₁₇ and/or X₁₉ which is either positively or negatively charged and is selected from natural amino acids, non-natural amino acids and derivatized amino acids.

13. (canceled)

14. A peptide according to claim 12, wherein
a) the negatively charged amino acid is selected from:
natural negatively charged amino acids;
non-natural negatively charged amino acids; and
originally positively charged amino acids which are derivatized with suitable chemical groups in order to provide them with a negatively charged group.
b) the positively charged amino acid is selected from:
natural Positively charged amino acids;
non-natural positively charged amino acids; and
originally negatively charged amino acids derivatized with suitable chemical groups in order to provide them with a positively charged group.

15-18. (canceled)

19. A peptide of at least 10 amino acids in length, capable of binding to the EPO receptor and comprising an agonist activity, selected from:
peptides comprising at least one core sequence of amino acids selected from:

| | |
|---|---|
| X₉X₁₀X₁₁X₁₂X₁₃; | (SEQ ID NO: 312) |
| X₉X₁₀X₁₁X₁₂X₁₃X₁₄X₁₅X₁₆X₁₇; | (SEQ ID NO: 313) |

and

| | |
|---|---|
| X₉X₁₀X₁₁X₁₂X₁₃X₁₄X₁₅X₁₆X₁₇X₁₈X₁₉; | (SEQ ID NO: 63) |

wherein each amino acid is selected from natural or non-natural amino acids and wherein in at least one of the positions X₁₀, X₁₇ or X₁₉ is a negatively charged amino acid and wherein
X₉ is G or a conservative exchange of G;
X₁₁ is selected from any amino acid;
X₁₂ is an uncharged polar amino acid or A;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid;
X₁₆ is independently selected from any amino acid;
X₁₈ is independently selected from any amino acid; and
functionally equivalent fragments, derivatives and variants of the peptides defined by the above consensus sequences, that depict an EPO mimetic activity and wherein in at least one of the positions X₁₀, X₁₇ or X₁₉ is a negatively charged amino acid.

20. A peptide according to claim 19, comprising the following sequence
(SEQ ID NO: 72)
X₆X₇X₈X₉X₁₀X₁₁X₁₂X₁₃X₁₄X₁₅X₁₆X₁₇X₁₈X₁₉
wherein each amino acid is selected from natural or non-natural amino acids and wherein
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
in case X₁₀ is not a negatively charged amino acid, X₁₀ is proline, a conservative exchange of proline or a non conservative exchange of proline or X₉ and X₁₀ are substituted by a single amino acid;
X₁₁ is selected from any amino acid;
X₁₂ is an uncharged polar amino acid or A;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid;
X₁₆ is independently selected from any amino acid;
in case X₁₇ is not a negatively charged amino acid, X₁₇ is selected from any amino acid;
X₁₈ is independently selected from any amino acid;
in case X₁₉ is not a negatively charged amino acid, X₁₉ is independently selected from any amino acid;
provided that at least one of X₁₀, X₁₇ or X₁₉ is a negatively charged amino acid.

21. A peptide according to claim 19, wherein the negatively charged amino acid is selected from:
natural negatively charged amino acids;
non-natural negatively charged amino acids; and
originally positively charged amino acids derivatized with suitable chemical groups in order to provide them with a negatively charged group.

22. A peptide according to claim 19, wherein the positively charged amino acid is present in at least one of the positions X₁₀, X₁₇ and/or X₁₉ and is selected from:
natural positively charged amino acids;
non-natural positively charged amino acids; and
originally negatively charged amino acids derivatized with suitable chemical groups in order to provide them with a positively charged group.

23-25. (canceled)

26. A peptide according to claim 19, selected from:
(SEQ ID NO: 116)
Ac-GGTYSCHFGKLT-Na1-VCKKQDG-Am
(SEQ ID NO: 117)
Ac-GGTYSCHFGKLT-Na1-VCKKQEG-Am
(SEQ ID NO: 118)
Ac-GGTYSCHFGKLT-Na1-VCKKQ-Aad-G-Am
(SEQ ID NO: 119)
Ac-GGTYSCHFGELT-Na1-VCKKQRG-Am
(SEQ ID NO: 120)
Ac-GGTYSCHFGDLT-Na1-VCKKQRG-Am
(SEQ ID NO: 121)
Ac-GGTYSCHFGKLT-Na1-VCKEQRG-Am
(SEQ ID NO: 122)
Ac-GGTYSCHFGKLT-Na1-VCKDQRG-Am
(SEQ ID NO: 126)
Ac-GGTYSCHFGKLT-Na1-VCK-K(Glr)-QRG-Am
(SEQ ID NO: 127)
Ac-GGTYSCHFGKLT-Na1-VCK-K(Adi)-QRG-Am
(SEQ ID NO: 297)
Ac-GATYSCHFGKLT-Na1-VCKKQ-Aad-G-Am
(SEQ ID NO: 298)
Ac-GGTYSCHFGKLT-Na1-VCK-Har-QDG-Am
(SEQ ID NO: 299)
Ac-GGTYSCHFGKLT-Na1-VCK-Har-Q-Aad-G-Am
(SEQ ID NO: 300)
GGGTYSCHFGKLT-Na1-VCKKQEG-Am
(SEQ ID NO: 301)
GGGTYSCHFGKLT-Na1-VCKKQ-Aad-G-Am

27-54. (canceled)

55. A peptide of at least 10 amino acids in length, capable of binding to the EPO receptor and comprising an agonist activity, selected from:
(a) a peptide, comprising the following core sequence of amino acids:

| | |
|---|---|
| X₉X₁₀X₁₁X₁₂X₁₃. | (SEQ ID NO: 314) |
| X₉X₁₀X₁₁X₁₂X₁₃X₁₄X₁₅X₁₆X₁₇ | (SEQ ID NO: 315) |
| or | |
| X₉X₁₀X₁₁X₁₂X₁₃X₁₄X₁₅X₁₆X₁₇X₁₈X₁₉ | (SEQ ID NO: 316) |

wherein each amino acid is selected from natural or non-natural amino acids, and wherein:
X₉ is G or a conservative exchange of G;
X₁₁ is selected from any amino acid;
X₁₂ is an uncharged polar amino acid or A;
X₁₃ is W, naphthylalanine, A or F;
X₁₄ is D, E, I, L or V;
X₁₅ is an amino acid with a sidechain functionality capable of forming a covalent bond or A or α-amino-γ-bromobutyric acid,
and functionally equivalent fragments, derivatives and variants of the peptides defined by the above consensus sequence, that depict an EPO mimetic activity,
wherein at least one of the positions X₁₀, X₁₆, X₁₇ or X₁₉ depicts a positively charged non-proteinogenic amino acid having a side chain which is elongated compared to lysine;
(b) a peptide, especially one being capable of binding the EPO receptor comprising the following sequence of amino acids:

| | |
|---|---|
| X₆X₇X₈X₉X₁₀X₁₁X₁₂X₁₃X₁₄X₁₅ | (SEQ ID NO: 317) |

wherein each amino acid is selected from natural or unnatural amino acids and X₆ is C, A, E, α-amino-γ-bromobutyric acid or homocysteine (hoc);
X₇ is R, H, L, W or Y or S;
X₈ is M, F, I, homoserinemethylether or norisoleucine;
X₉ is G or a conservative exchange of G;
X₁₀ is Har
X₁₁ is selected from any amino acid;
X₁₂ is T or A;
X₁₃ is W, 1-nal, 2-nal, A or F;
X₁₄ is D, E, I, L or V;
X₁₅ is C, A, K, α-amino-γ-bromobutyric acid or homocysteine (hoc) provided that either X₆ or X₁₅ is C or hoc; and
(c) a peptide comprising:
(SEQ ID NO: 75)
X₃X₄X₅X₆X₇X₈X₉X₁₀X₁₁X₁₂X₁₃X₁₄X₁₅X₁₆X₁₇X₁₈
wherein X₆ to X₁₅ have the above meaning of variant (b) and wherein
X₃ is independently selected from any amino acid;
X₄ is Y;
X₅ is independently selected from any amino acid;
X₁₆ is independently selected from any amino acid;
X₁₇ is homoarginine;
X₁₈ is independently selected from any amino acid.

56. A peptide according to claim 55, comprising the following core sequence of amino acids:
(SEQ ID NO: 72)
X₆X₇X₈X₉X₁₀X₁₁X₁₂X₁₃X₁₄X₁₅X₁₆X₁₇X₁₈X₁₉
wherein each amino acid is selected from natural or non-natural amino acids and wherein
X₆ is an amino acid with a sidechain functionality capable of forming a covalent bond or A or α-amino-γ-bromobutyric acid;
X₇ is R, H, L, W or Y or S;
X₈ is M, F, I, Y, H, homoserinemethylether or norisoleucine;
X₉ is G or a conservative exchange of G;
in case X₁₀ is not a positively charged non-proteinogenic amino acid having a side chain which is elongated compared to lysine, X₁₀ is proline, a conservative exchange of proline or a non conservative exchange of proline or X₉ and X₁₀ are substituted by a single amino acid;
X₁₁ is selected from any amino acid;
X₁₂ is an uncharged polar amino acid or A;
X₁₃ is W, 1-nal, 2-nal, A or F;
X₁₄ is D, E, I, L or V;
X₁₅ is an amino acid with a sidechain functionality capable of forming a covalent bond or A or α-amino-γ-bromobutyric acid;
in case X₁₆ is not a positively charged non-proteinogenic amino acid having a side chain which is elongated compared to lysine, X₁₆ is independently selected from any amino acid;
in case X₁₇ is not a positively non-proteinogenic charged amino acid having a side chain which is elongated compared to lysine, X₁₇ is selected from any amino acid;
X₁₈ is independently selected from any amino acid, preferably L or Q;
in case X₁₉ is not a positively charged non-proteinogenic amino acid having a side chain which is elongated compared to lysine, X₁₉ is independently selected from any amino acid;
provided that at least one of X₁₀, X₁₆, X₁₇ or X₁₉ is a positively charged non-proteinogenic amino acid having a side chain which is elongated compared to lysine.

57. A peptide according to claim 13, wherein at least one of X₁₀, X₁₆, X₁₇ or X₁₉ is a positively charged amino acid and wherein the positively charged amino acid is selected from:
natural positively charged amino acids;
non-natural positively charged amino acids;
originally negatively charged amino acids derivatized with suitable chemical groups in order to provide them with a positively charged group;
provided that at least one of X₁₀, X₁₆, X₁₇ or X₁₉ is a positively charged non-proteinogenic amino acid having a side chain which is elongated compared to lysine.

58-64. (canceled)

65. A peptide according to claim 57, wherein X₁₀, X₁₇ and/or X₁₉ is a negatively charged amino acid and wherein said negatively charged amino acid is selected from:
natural negatively charged amino acids;
non-natural negatively charged amino acids; and
originally positively charged amino acids derivatized with suitable chemical groups in order to provide them with a negatively charged group.

66-73. (canceled)

74. A compound binding to target molecules, comprising
(i) at least two peptide units wherein each peptide unit comprises at least two domains with a binding capacity to a target; and
(ii) at least one polymeric carrier unit;
wherein said peptide units are attached to said polymeric carrier unit and wherein at least one domain of at least one peptide unit is a peptide according to claim 1.

75. The compound according to claim 74, wherein at least one peptide unit comprises a peptide dimer comprising at least one monomeric peptide consensus sequence according to claim 1.

76-87. (canceled)

88. A method for dimerizing monomeric peptide units to form an EPO mimetic peptide dimer, wherein the dimer is created by forming a covalent bond between the monomeric peptide units, wherein said bond is formed between the C-terminal amino acid of the first monomeric peptide unit and the N-terminal amino acid of the second monomeric peptide unit.

89. The method according to claim 88, wherein monomeric peptide units are used, carrying an amino acid at either the C— or the N-terminus with a side chain functionality capable of forming a covalent bond, wherein a covalent bond is formed between the side chain of the C-terminal amino acid of the first monomeric peptide unit and the side chain of the N-terminal amino acid of the second monomeric peptide unit.

90-93. (canceled)
